(19) United States
(12) Patent Application Publication
(10) Pub. No.: US 2010/0286173 A1
(43) Pub. Date: Nov. 11, 2010

(54) FUNGICIDE HYDROXIMOYL-TETRAZOLE DERIVATIVES

(76) Inventors: Christian Beier, Saint Genis-Laval (FR); Jurgen Benting, Leichlingen (DE); Isabelle Christian, Lyon (FR); Pierre-Yves Coqueron, Lyon (FR); Christophe Duboist, Lyon (FR); Pierre Genix, Lyon (FR); Marie-Claire Grosjean-Cournoyer, Curis Au Mont D'Or (FR); Benoit Hartmann, Sainte Foy-Les-Lyon (FR); Pierre Hutin, Lyon (FR); Daniela Portz, Vettweis (DE); Philippe Rinolfi, Chatillon D'Azergues (FR); Arnd Voerste, Koln (DE); Jean-Pierre Vors, Sainte Foy-Les-Lyon (FR); Ulrike Wachendorff-Neumann, Neuwied (DE)

Correspondence Address: OSTROLENK FABER GERB & SOFFEN, 1180 AVENUE OF THE AMERICAS, NEW YORK, NY, 100368403, US

(21) Appl. No.: 12/735,434

(22) PCT Filed: Jan. 16, 2009
(86) PCT No.: PCT/EP2009/050482

§ 371 Date: Jul. 16, 2010

(30) Foreign Application Priority Data
Jan. 16, 2008 (EP) 08356009.4

(51) Int. Cl. A01N 43/713; C07D 401/12; A01P 3/00; C07D 403/12; C07D 417/12; A01N 43/78

(52) U.S. Cl. 514/256; 546/268.4; 514/340; 544/328; 548/197; 514/370; 514/371; 548/196

(57) ABSTRACT

The present invention relates to hydroximoyl-tetrazole derivatives, their process of preparation, their use as fungicide active agents, particularly in the form of fungicide compositions, and methods for the control of phytopathogenic fungi, notably of plants, using these compounds or compositions.

The present invention relates to hydroximoyl-tetrazole derivatives, their process of preparation, their use as fungicide active agents, particularly in the form of fungicide compositions, and methods for the control of phytopathogenic fungi, notably of plants, using these compounds or compositions.

In European patent application n°1426371, there are disclosed certain tetrazolyloxime derivatives of the following chemical structure:

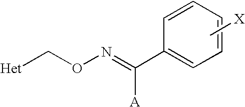

wherein A represents a tetrazolyl group, Het represents either a particular pyridinyl group or a particular thiazolyl group.

In Japanese patent application n°2004-131392, there are disclosed certain tetrazolyloxime derivatives of the following chemical structure:

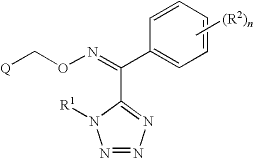

wherein Q can be selected in a list of 15 various heterocycle groups.

The compounds disclosed in these two documents do not prove to provide a comparable utility than the compounds according to the invention.

It is always of high-interest in agriculture to use novel pesticide compounds in order to avoid or to control the development of resistant strains to the active ingredients. It is also of high-interest to use novel compounds being more active than those already known, with the aim of decreasing the amounts of active compound to be used, whilst at the same time maintaining effectiveness at least equivalent to the already known compounds. We have now found a new family of compounds which possess the above mentioned effects or advantages.

Accordingly, the present invention provides hydroximoyl-tetrazole derivatives of formula (I)

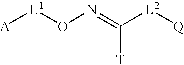

wherein
T represents a substituted or non-substituted tetrazolyl group;
L¹ represents a direct bond or a divalent group selected in the list consisting of
   —(CR¹Z²)ₙ— —(CR¹R²)ₘ—C(═O)—(CR¹R²)ₚ—
   —(CR¹R²)ₘ—(CR¹═CR²)—(CR¹R²)ₚ— —(CR¹R²)ₘ—C(═O)—O—(CR¹R²)ₚ
   —(CR¹R²)ₘ—C≡C—(CR¹R²)ₚ— —(CR¹R²)ₘ—O—C(═O)—(CR¹R²)ₚ—
   —(CR¹R²)ₘ—O—(CR¹R²)ₚ— —(CR¹R²)ₘ—C(═O)—NH—(CR¹R²)ₚ—
   —(CR¹R²)ₘ—NH—(CR¹R²)ₚ— —(CR¹R²)ₘ—NH—C(═O)—(CR¹R²)ₚ—
      wherein
n represents 1, 2, 3 or 4;
m and p independently represent 0, 1, 2 or 3;
L² represents a direct bond or a divalent group selected in the list consisting of
   —(CR³R⁴)_{q}— —(CR²R⁴)ₐ—C(═O)—(CR³R⁴)_{b}—
   —(CR³R⁴)ₐ—(CR³═CR⁴)—(CR³R⁴)_{b}— —(CR³R⁴)ₐ—C(═O)—O—(CR³R⁴)_{b}
—(CR³R⁴)ₐ—C≡C—(CR³R⁴)_{b}— —(CR³R⁴)ₐ—O—C(O)—(CR³R⁴)_{b}—
   —(CR³R⁴)ₐ—O—(CR³R⁴)_{b}— —(CR³R⁴)ₐ—C(═O)—NH—(CR³R⁴)_{b}—
   —(CR³R⁴)ₐ—NH—(CR³R⁴)_{b}— —(CR³R⁴)ₐ—NH—C(═O)—(CR³R⁴)_{b}—
      wherein
q represents 1, 2, 3 or 4;
a and b independently represent 0, 1, 2 or 3;
A is selected in the list consisting of A¹ to A¹¹⁶

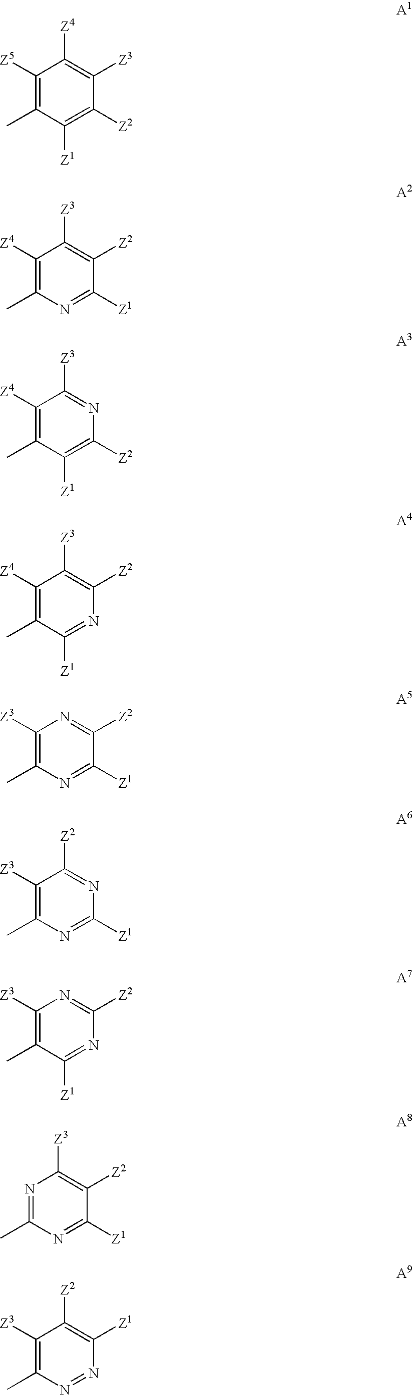

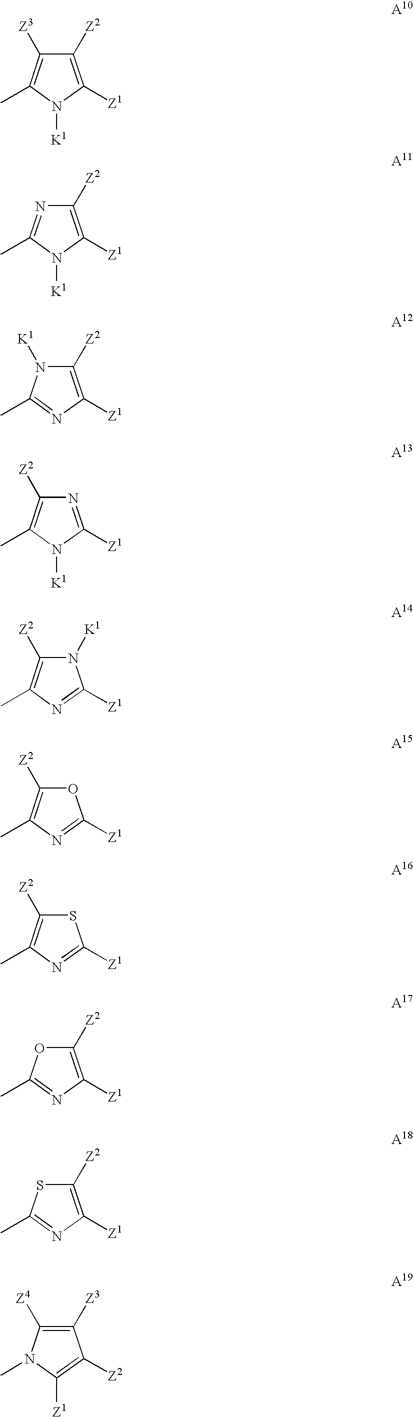

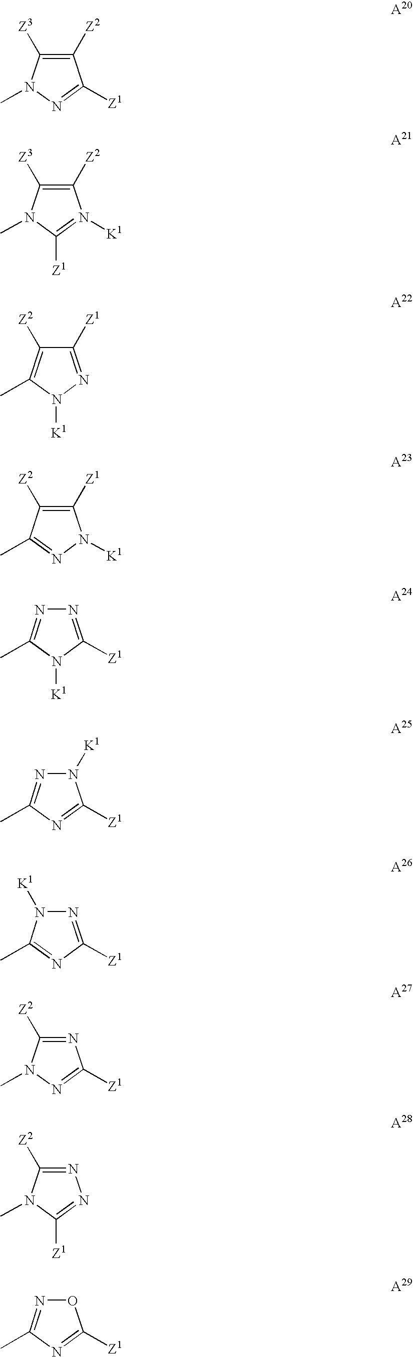

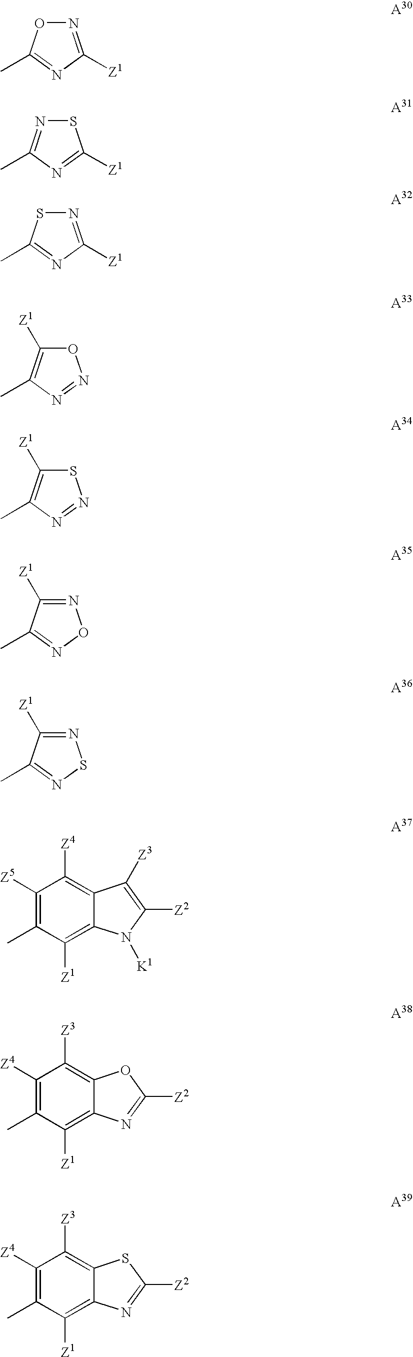

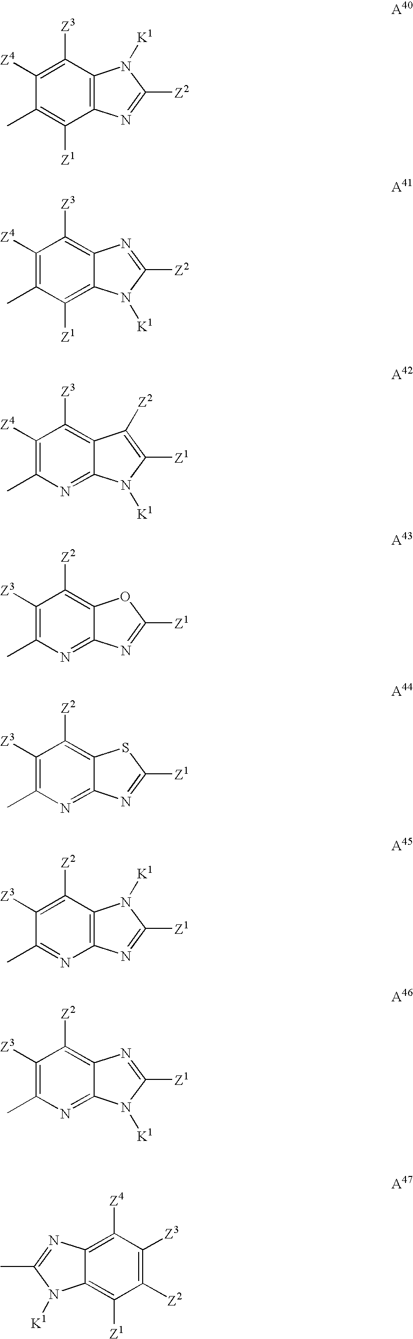

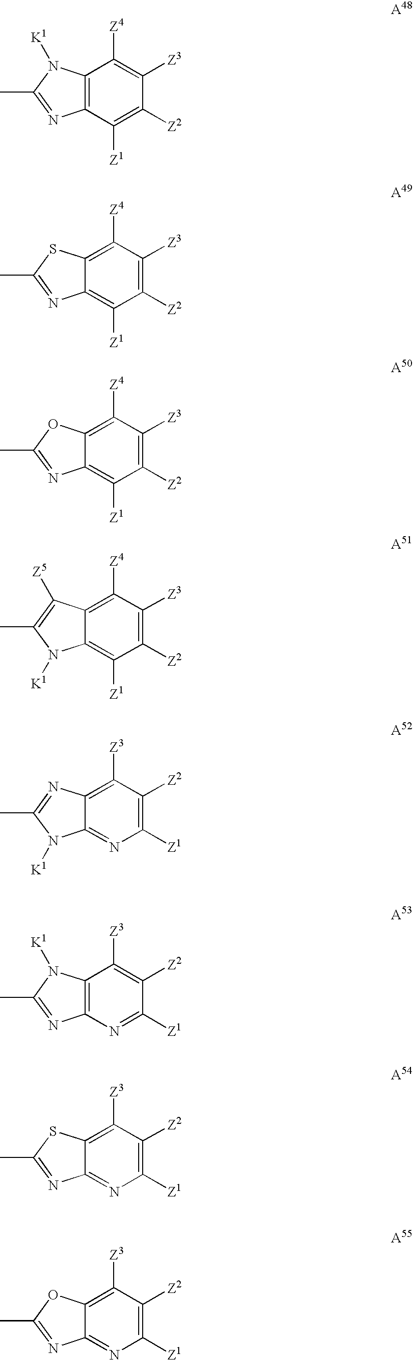

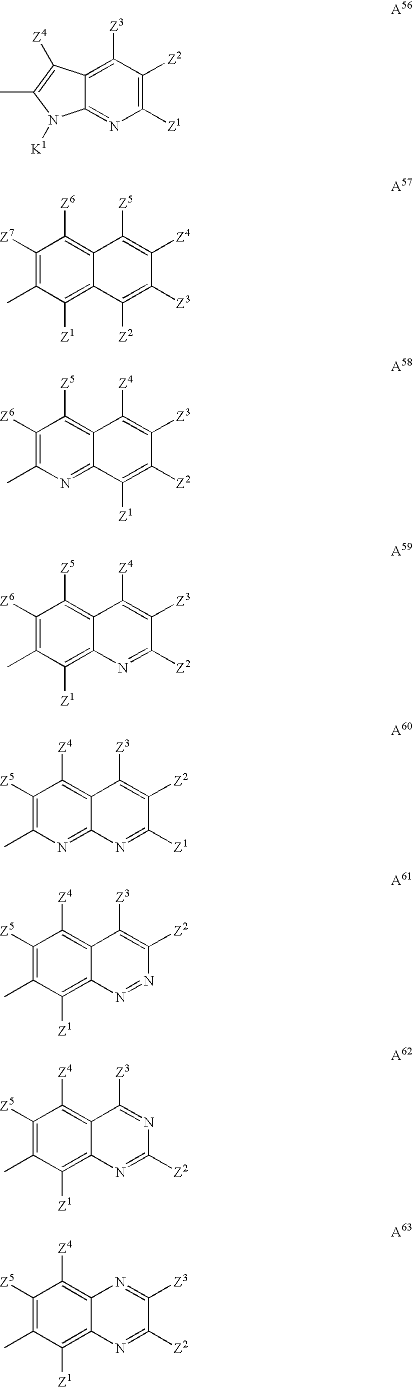

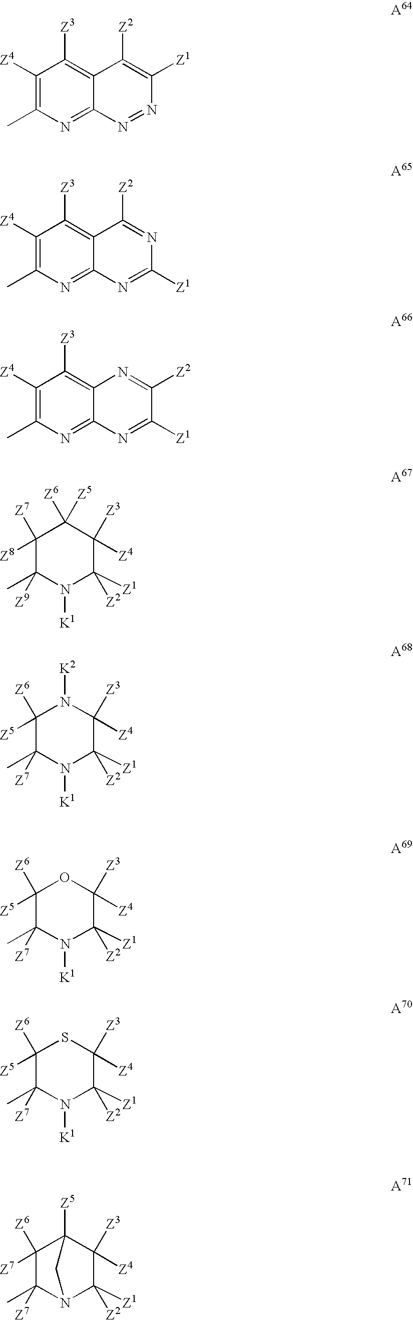

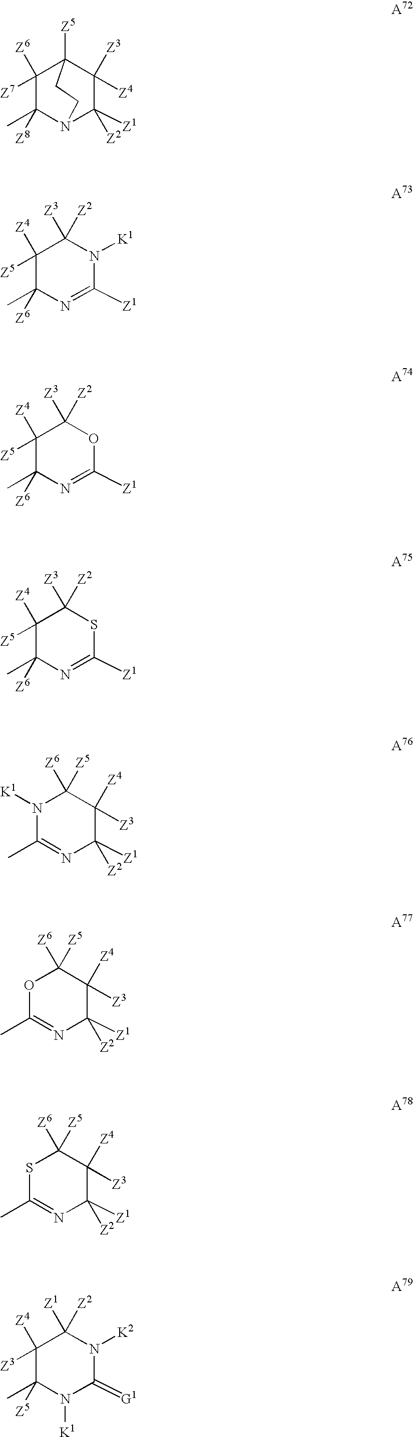

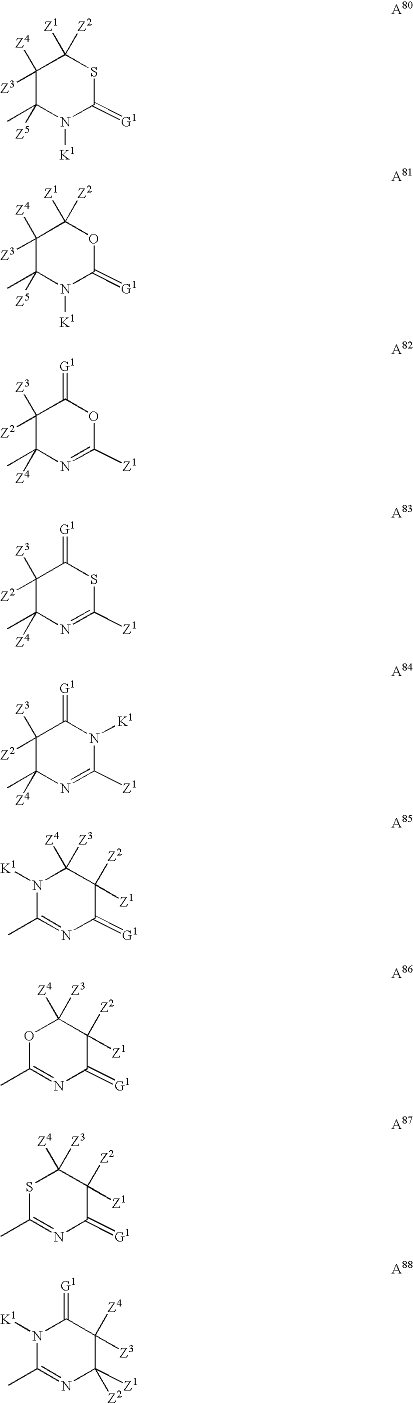

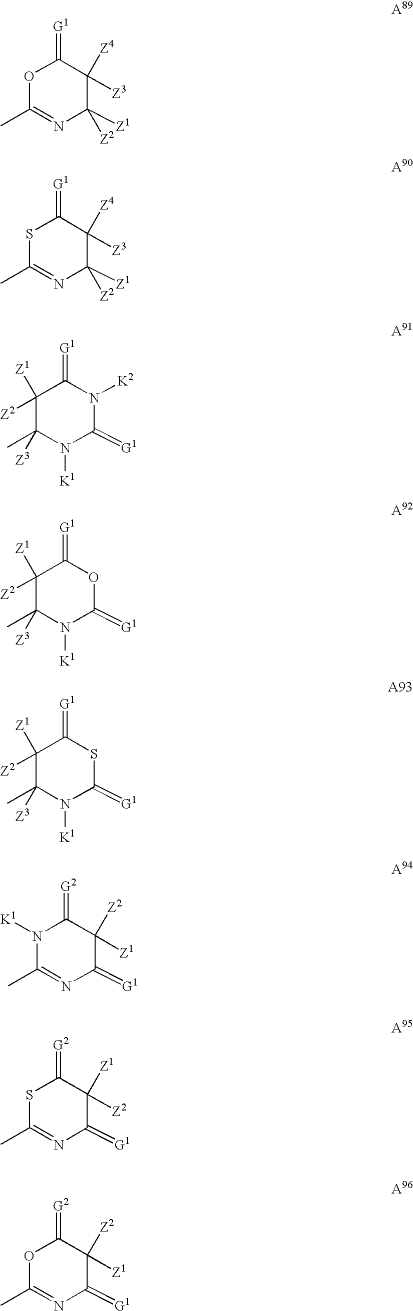

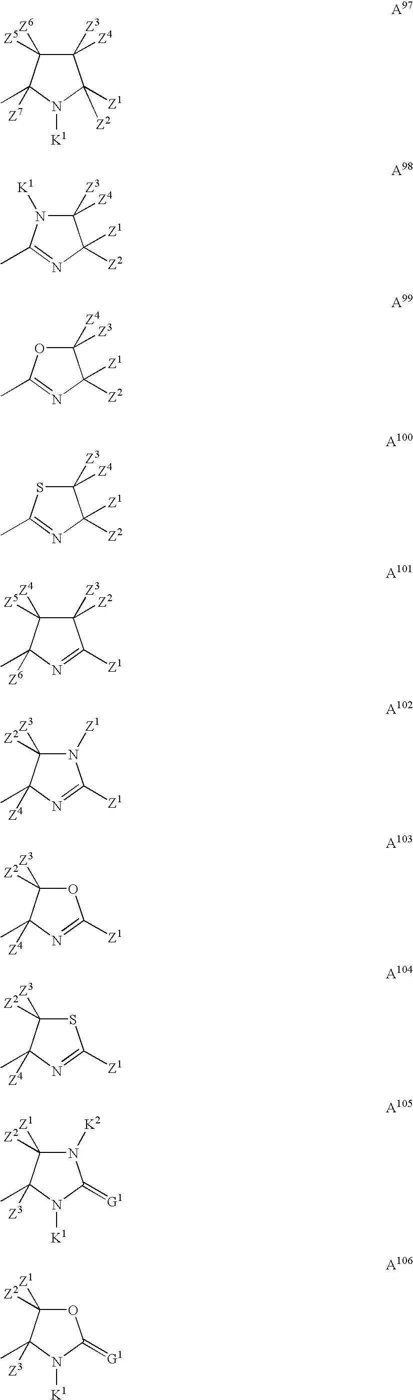

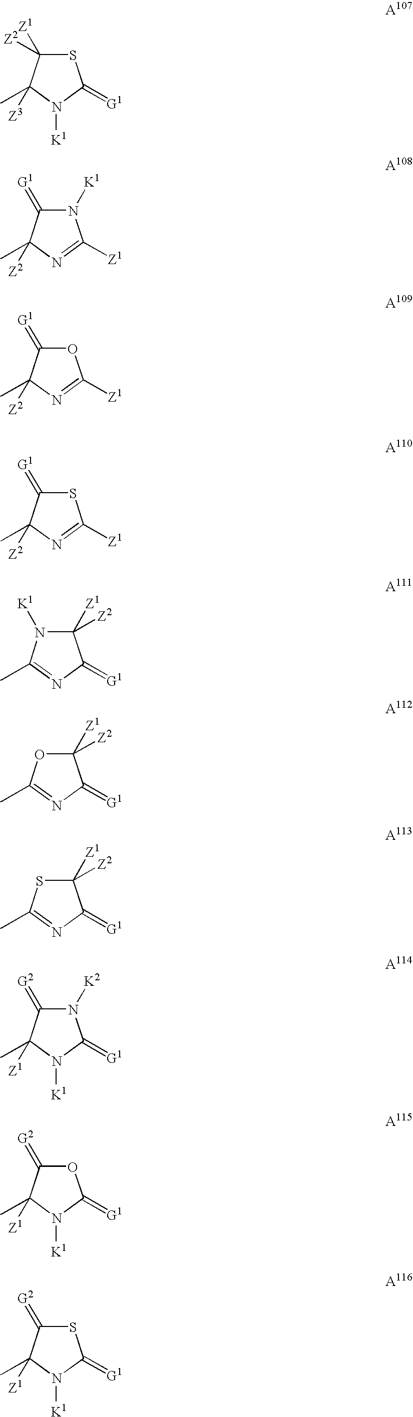

wherein
Z¹, Z², Z³, Z⁴, Z⁵, Z⁶, Z⁷, Z⁸ and Z⁹ independently represent a hydrogen atom, a halogen atom, a nitro group, a hydroxy group, a cyano group, an amino group, a sulphenyl group, a formyl group, a substituted or non-substituted carbaldehyde O—(C₁-C₈-alkyl)oxime, a formyloxy group, a formylamino group, a carbamoyl group, a N-hydroxycarbamoyl group, a pentafluoro-λ⁶-sulphenyl group, a formylamino group, substituted or non-substituted C₁-C₈-alkoxyamino group, substituted or non-substituted N—C₁-C₈-alkyl-(C₁-C₈-alkoxy)-amino group, substituted or non-substituted (C₁-C₈-alkylamino)-amino group, substituted or non-substituted N—C₁-C₈-alkyl-(C₁-C₈-alkylamino)-amino group, a substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C3-C8-cycloalkyl, substituted or non-substituted tri(C1-C8-alkyl)silyl-C3-C8-cycloalkyl, substituted or non-substituted C₁-C₈-halogenoalkyl having 1 to 5 halogen atoms, substituted or non-substituted C3-C8-halogenocycloalkyl having 1 to 5 halogen atoms, a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted (C₁-C₈-alkoxycarbonyl)amino, substituted or non-substituted (C₃-C₈-cycloalkoxycarbonyl)amino, substituted or non-substituted C₁-C₈-halogenoalkoxy having 1 to 5 halogen atoms, substituted or non-substituted C₁-C₈-alkylsulphenyl, substituted or non-substituted C₁-C₈-halogenoalkylsulphenyl having 1 to 5 halogen atoms, substituted or non-substituted C₂-C₈-alkenyloxy, substituted or non-substituted (C₂-C₈-alkenyloxycarbonyl)amino, substituted or non-substituted C₂-C₈-halogenoalkenyloxy having 1 to 5 halogen atoms, substituted or non-substituted C₃-C₈-alkynyloxy, substituted or non-substituted (C₃-C₈-alkenyloxycarbonyl)amino, substituted or non-substituted C₃-C₈-halogenoalkynyloxy having 1 to 5 halogen atoms, substituted or non-substituted C₁-C₈-alkylcarbonyl, substituted or non-substituted N—(C₁-C₈-alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N-C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈-alkylcarbamoylamino, substituted or non-substituted C₁-C₈-halogenoalkylcarbamoylamino having 1 to 5 halogen atoms, substituted or non-substituted di-C₁-C₈-alkylcarbamoylamino, substituted or non-substituted di-C₁-C₈-halogenoalkylcarbamoylamino having 1 to 5 halogen atoms, substituted or non-substituted N—C₁-C₈-alkyl-(C₁-C₈-alkylcarbamoyl)amino, substituted or non-substituted N—C₁-C₈-alkyl-(C₁-C₈-halogenoalkylcarbamoyl)amino having 1 to 5 halogen atoms, substituted or non-substituted N—C₁-C₈-alkyl-(di-C₁-C₈-alkylcarbamoyl)amino, substituted or non-substituted N—C₁-C₈-alkyl-(di-C₁-C₈-halogenoalkylcarbamoyl)amino having 1 to 5 halogen atoms, substituted or non-substituted C₁-C₈-alkylaminocarbonyloxy, substituted or non-substituted di-C₁-C₈-alkylaminocarbonyloxy, substituted or non-substituted C₁-C₈-alkylcarbamothioyl, substituted or non-substituted di-C₁-C₈-alkylcarbamothioyl, substituted or non-substituted N—C₁-C₈-alkyloxycarbamothioyll, substituted or non-substituted C₁-C₈-alkoxycarbamothioyl, substituted or non-substituted N—C₁-C₈-alkyl-C₁-C₈-alkoxycarbamothioyll, substituted or non-substituted C₁-C₈-alkylthioylamino, substituted or non-substituted (C₁-C₈-alkoxythiocarbonyl)amino, substituted or non-substituted C₁-C₈-halogenoalkylthioylamino having 1 to 5 halogen atoms, substituted or non-substituted (C₁-C₈-alkyl-carbamothioyl)-oxy, substituted or non-substituted substituted or non-substituted (di-C₁-C₈-alkyl-carbamothioyl)-oxy, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted C₁-C₈-alkylaminosulfamoyl, substituted or non-substituted di-C₁-C₈-alkylaminosulfamoyl, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, substituted or non-substituted (benzyloxyimino)-C₁-C₆-alkyl, substituted or non-substituted benzyloxy, substituted or non-substituted benzylsulphenyl, substituted or non-substituted benzylamino, substituted or non-substituted phenoxy, substituted or non-substituted phenylsulphenyl, substituted or non-substituted phenylamino, substituted or non-substituted aryl, substituted or non-substituted (arylcarbonyl)amino, substituted or non-substituted (heterocyclylcarbonyl)amino substituted or non-substituted aryl-[C₁-C₈]-alkyl, substituted or non-substituted tri(C₁-C₈-alkyl)-silyloxy, substituted or non-substituted C₁-C₈-alkylsulfenylamino, substituted or non-substituted C₁-C₈-halogenoalkylsulphinylamino having 1 to 5 halogen atoms, substituted or non-substituted C₁-C₈-alkylsulphonylamino, substituted or non-substituted C₁-C₈-halogenoalkylsulphonylamino having 1 to 5 halogen atoms, substituted or non-substituted C₁-C₈-alkoxysulphonylamino, substituted or non-substituted C₁-C₈-halogenoxysulphonylamino having 1 to 5 halogen atoms, substituted or non-substituted tri(C₁-C₈alkyl)-silyl, substituted or non-substituted (C₁-C₆-alkylideneamino)oxy, substituted or non-substituted (C₁-C₆-alkenylideneamino)oxy, substituted or non-substituted (C₁-C₆-alkynylideneamino)oxy, substituted or non-substituted (benzylideneamino)oxy;
   provided that when A represents A² or A¹⁶ and when Z¹, Z², Z³, Z⁴, Z⁵, Z⁶, Z⁷, Z⁸ and Z⁹ independently represent a substituted C₁-C₈-alkylamino or substituted benzylamino, then the substituant is chosen in the list consisting of a pentafluoro-λ⁶-sulphenyl group, a substituted or non-substituted carbaldehyde O—(C₁-C₈-alkyl)oxime, a formylamino group, a (hydroxyimino)-C₁-C₆-alkyl group, a tri(C₁-C₈-alkyl)silyl-C₁-C₈-alkyl, C₃-C₈-cycloalkyl, tri(C₁-C₈alkyl)silyl-C₁-C₈-cycloalkyl, a C₁-C₈-halogenocycloalkyl having 1 to 5 halogen atoms, C₃-C₈-cycloalkoxy, a C₂-C₈-halogenoalkenyloxy having 1 to 5 halogen atoms, a C₃-C₈-halogenoalkynyloxy having 1 to 5 halogen atoms, a (C₁-C₆-alkoxyimino)-C₁-C₆-alkyl, a (C₁-C₆-alkenyloxyimino)-C₁-C₆-alkyl, a (C₁-C₆-alkynyloxyimino)-C₁-C₆-alkyl, and (benzyloxyimino)-C₁-C₆-alkyl.
K¹ and K² independently represent a hydrogen atom, a formyl group, a substituted or non-substituted carbaldehyde O—(C₁-C₈-alkyl)oxime, a carbamoyl group, a N-hydroxycarbamoyl group, a formylamino group, substituted or non-substituted C₁-C₈-alkyl, substituted or non-substituted tri(C₁-C₈-alkyl)silyl-C₁-C₈-alkyl, substituted or non-substituted C₃-C₈-cycloalkyl, substituted or non-substituted tri(C₁-C₈-alkyl)silyl-C₃-C₈-cycloalkyl, substituted or non-substituted C₁-C₈-halogenoalkyl having 1 to 5 halogen atoms, substituted or non-substituted C₃-C₈-halogenocycloalkyl having 1 to 5 halogen atoms, a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₃-alkoxy, substituted or non-substituted C₁-C₈-halogenoalkoxy having 1 to 5 halogen atoms, substituted or non-substituted C₂-C₈alkenyloxy, substituted or non-substituted C₂-C₈-halogenoalkenyloxy having 1 to 5 halogen atoms, substituted or non-substituted C₂-C₈-alkynyloxy, substituted or non-substituted C₃-C₈-halogenoalkynyloxy having 1 to 5 halogen atoms, substituted or non-substituted C₁-C₈alkylcarbonyl, substituted or non-substituted N—(C₁-C₈-alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbamothioyl, substituted or non-substituted di-C₁-C₈-alkylcarbamothioyl, substituted or non-substituted N—C₁-C₈-alkyloxycarbamothioyll, substituted or non-substituted C₁-C₈-alkoxycarbamothioyl, substituted or non-substituted N—C₁-C₈-alkyl-C₁-C₈-alkoxycarbamothioyl, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted C₁-C₈-alkylaminosulfamoyl, substituted or non-substituted di-C₁-C₈-alkylaminosulfamoyl, substituted or non-substituted aryl, substituted or non-substituted aryl-[C₁-C₈]-alkyl;
G¹ and G² are independently selected in the list consisting of oxygen, sulfur, NR⁵, N—OR⁶ and N—NR⁷R⁸;
Q represents a substituted phenyl group of formula (VI)

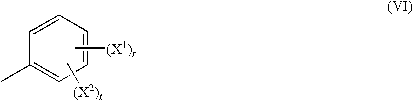

wherein:
X¹ independently represents a nitro group, a hydroxy group, a cyano group, an amino group, a sulphenyl group, a formyl group, a substituted or non-substituted carbaldehyde O—(C₁-C₈-alkyl)oxime, a formyloxy group, a formylamino group, a carbamoyl group, a N-hydroxycarbamoyl group, a pentafluoro-λ⁶-sulphenyl group, a formylamino group, substituted or non-substituted C₁-C₈-alkoxyamino group, substituted or non-substituted N—C₁-C₈-alkyl-(C₁-C₈-alkoxy)-amino group, substituted or non-substituted (C₁-C₈-alkylamino)-amino group, substituted or non-substituted N—C₁-C₈-alkyl-(C₁-C₈-alkylamino)-amino group, a substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C₃-C₈-cyclo alkyl, substituted or non-substituted tri(C₁-C₈-alkyl)silyl-C₃-C₈-cycloalkyl, substituted or non-substituted C₁-C₈-halogenoalkyl having 1 to 5 halogen atoms, substituted or non-substituted C₃-C₈-halogenocycloalkyl having 1 to 5 halogen atoms, a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms, substituted or non-substituted C₁-C₈-alkylsulphenyl, substituted or non-substituted C₁-C₈-halogenoalkylsulphenyl having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms, substituted or non-substituted C₁-C₈-alkylcarbonyl, substituted or non-substituted N—(C₁-C₈-alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈-alkylcarbamoylamino, substituted or non-substituted C₁-C₈-halogenoalkylcarbamoylamino having 1 to 5 halogen atoms, substituted or non-substituted di-C₁-C₈-alkylcarbamoylamino, substituted or non-substituted di-C₁-C₈-halogenoalkylcarbamoylamino having 1 to 5 halogen atoms, substituted or non-substituted N—C₁-C₈-alkyl-(C₁-C₈-alkylcarbamoyl)amino, substituted or non-substituted N—C₁-C₈-alkyl-(C₁-C₈-halogenoalkylcarbamoyl)amino having 1 to 5 halogen atoms, substituted or non-substituted N—C₁-C₈-alkyl-(di-C₁-C₈-alkylcarbamoyl)amino, substituted or non-substituted N—C₁-C₈-alkyl-(di-C₁-C₈-halogenoalkylcarbamoyl)amino having 1 to 5 halogen atoms, substituted or non-substituted C₁-C₈-alkylaminocarbonyloxy, substituted or non-substituted di-C₁-C₈-alkylaminocarbonyloxy, substituted or non-substituted C₁-C₈-alkylcarbamothioyl, substituted or non-substituted di-C₁-C₈-alkylcarbamothioyl, substituted or non-substituted N—C₁-C₈-alkyloxycarbamothioyll, substituted or non-substituted C₁-C₈-alkoxycarbamothioyl, substituted or non-substituted N—C₁-C₈-alkyl-C₁-C₈-alkoxycarbamothioyll, substituted or non-substituted C₁-C₈-alkylthioylamino, substituted or non-substituted C₁-C₈-halogenoalkylthioylamino having 1 to 5 halogen atoms, substituted or non-substituted (C₁-C₈-alkyl-carbamothioyl)-oxy, substituted or non-substituted substituted or non-substituted (di-C₁-C₈-alkyl-carbamothioyl)-oxy, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted C₁-C₈-alkylaminosulfamoyl, substituted or non-substituted di-C₁-C₈-alkylamino sulfamoyl, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, substituted or non-substituted (benzyloxyimino)-C₁-C₆-alkyl, substituted or non-substituted benzyloxy, substituted or non-substituted benzylsulphenyl, substituted or non-substituted benzylamino, substituted or non-substituted phenoxy, substituted or non-substituted phenylsulphenyl, substituted or non-substituted phenylamino, substituted or non-substituted aryl, substituted or non-substituted aryl-[C₁-C₈]-alkyl, substituted or non-substituted tri(C₁-C₈-alkyl)-silyloxy, substituted or non-substituted C₁-C₈-alkylsulfenylamino, substituted or non-substituted C₁-C₈-halogenoalkylsulphinylamino having 1 to 5 halogen atoms, substituted or non-substituted C₁-C₈-alkylsulphonylamino, substituted or non-substituted C₁-C₈-halogenoalkylsulphonylamino having 1 to 5 halogen atoms, substituted or non-substituted C₁-C₈-alkoxysulphonylamino, substituted or non-substituted C₁-C₈-halogenoxysulphonylamino having 1 to 5 halogen atoms, substituted or non-substituted tri(C₁-C₈-alkyl)-silyl, substituted or non-substituted (C₁-C₆-alkylideneamino)oxy, substituted or non-substituted (C₁-C₆-alkenylideneamino)oxy, substituted or non-substituted (C₁-C₆-alkynylideneamino)oxy, substituted or non-substituted (benzylideneamino)oxy; and
X² independently represents a halogen atom, a nitro group, a hydroxy group, a cyano group, an amino group, a sulphenyl group, a formyl group, a substituted or non-substituted carbaldehyde O—(C₁-C₈-alkyl)oxime, a formyloxy group, a formylamino group, a carbamoyl group, a N-hydroxycarbamoyl group, a pentafluoro-λ⁶-sulphenyl group, a formylamino group, substituted or non-substituted C₁-C₈-alkoxyamino group, substituted or non-substituted N—C₁-C₈-alkyl-(C₁-C₈-alkoxy)-amino group, substituted or non-substituted (C₁-C₈-alkylamino)-amino group, substituted or non-substituted N—C₁-C₈-alkyl-(C₁-C₈-alkylamino)-amino group, a substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C₃-C₈-cycloalkyl, substituted or non-substituted tri(C₁-C₈-alkyl)silyl-C₃-C₈-cycloalkyl, substituted or non-substituted C₁-C₈-halogenoalkyl having 1 to 5 halogen atoms, substituted or non-substituted C₃-C₈-halogenocycloalkyl having 1 to 5 halogen atoms, a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms, substituted or non-substituted C₁-C₈-alkylsulphenyl, substituted or non-substituted C₁-C₈-halogenoalkylsulphenyl having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms, substituted or non-substituted C₁-C₈-alkylcarbonyl, substituted or non-substituted N—(C₁-C₈-alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈alkylcarbamoylamino, substituted or non-substituted C₁-C₈-halogenoalkylcarbamoylamino having 1 to 5 halogen atoms, substituted or non-substituted di-C₁-C₈-alkylcarbamoylamino, substituted or non-substituted di-C₁-C₈-halogenoalkylcarbamoylamino having 1 to 5 halogen atoms, substituted or non-substituted N-C₁-C₈-alkyl-(C₁-C₈-alkylcarbamoyl)amino, substituted or non-substituted N—C₁-C₈-alkyl-(C₁-C₈-halogenoalkylcarbamoyl)amino having 1 to 5 halogen atoms, substituted or non-substituted N—C₁-C₈-alkyl-(di-C₁-C₈-alkylcarbamoyl)amino, substituted or non-substituted N—C₁-C₈alkyl-(di-C₁-C₈-halogenoalkylcarbamoyl)amino having 1 to 5 halogen atoms, substituted or non-substituted C₁-C₈-alkylaminocarbonyloxy, substituted or non-substituted di-C₁-C₈-alkylaminocarbonyloxy, substituted or non-substituted C₁-C₈-alkylcarbamothioyl, substituted or non-substituted di-C₁-C₈-alkylcarbamothioyl, substituted or non-substituted N—C₁-C₈-alkyloxycarbamothioyll, substituted or non-substituted C₁-C₈-alkoxycarbamothioyl, substituted or non-substituted N—C₁-C₈-alkyl-C₁-C₈-alkoxycarbamothioyll, substituted or non-substituted C₁-C₈-alkylthioylamino, substituted or non-substituted C₁-C₈-halogenoalkylthioylamino having 1 to 5 halogen atoms, substituted or non-substituted (C₁-C₈-alkyl-carbamothioyl)-oxy, substituted or non-substituted substituted or non-substituted (di-C₁-C₈-alkyl-carbamothioyl)-oxy, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted C₁-C₈-alkylaminosulfamoyl, substituted or non-substituted di-C₁-C₈-alkylaminosulfamoyl, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, substituted or non-substituted (benzyloxyimino)-C₁-C₆-alkyl, substituted or non-substituted benzyloxy, substituted or non-substituted benzylsulphenyl, substituted or non-substituted benzylamino, substituted or non-substituted phenoxy, substituted or non-substituted phenylsulphenyl, substituted or non-substituted phenylamino, substituted or non-substituted aryl, substituted or non-substituted aryl-[C₁-C₈]-alkyl, substituted or non-substituted tri(C₁-C₈-alkyl)-silyloxy, substituted or non-substituted C₁-C₈-alkylsulfenylamino, substituted or non-substituted C₁-C₈-halogenoalkylsulphinylamino having 1 to 5 halogen atoms, substituted or non-substituted C₁-C₈alkylsulphonylamino, substituted or non-substituted C₁-C₈-halogenoalkylsulphonylamino having 1 to 5 halogen atoms, substituted or non-substituted C₁-C₈-alkoxysulphonylamino, substituted or non-substituted C₁-C₈-halogenoxysulphonylamino having 1 to 5 halogen atoms, substituted or non-substituted tri(C₁-C₈-alkyl)-silyl, substituted or non-substituted (C₁-C₆-alkylideneamino)oxy, substituted or non-substituted (C₁-C₆-alkenylideneamino)oxy, substituted or non-substituted (C₁-C₈-alkynylideneamino)oxy, substituted or non-substituted (benzylideneamino)oxy; and
r represents 1, 2, 3, 4 or 5;
t represents 0, 1, 2, 3, 4 or 5 and the sum of r and t equals to 2, 3, 4 or 5;
R¹, R², R³ and R⁴ independently represent a hydrogen atom, a halogen atom, a cyano group, substituted or non-substituted C₁-C₈-alkyl, substituted or non-substituted C₃-C₈-cycloalkyl, substituted or non-substituted C₁-C₈-halogenoalkyl having 1 to 5 halogen atoms, substituted or non-substituted C₃-C₈-halogenocycloalkyl having 1 to 5 halogen atoms, a C₂-C₈-alkenyl, substituted or non-substituted C₂-C₈-alkynyl, substituted or non-substituted C₁-C₈-alkoxy, substituted or non-substituted C₁-C₈-halogenoalkoxy having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈alkynyloxy, substituted or non-substituted C₃-C₈-halogenoalkynyloxy having 1 to 5 halogen atoms,
R⁵ to R⁸ independently represent a hydrogen atom, a halogen atom, a cyano group, substituted or non-substituted C₁-C₈-alkyl, substituted or non-substituted C₃-C₈-cycloalkyl, substituted or non-substituted C₁-C₈-halogenoalkyl having 1 to 5 halogen atoms, substituted or non-substituted C₃-C₈-halogenocycloalkyl having 1 to 5 halogen atoms, a C₂-C₈-alkenyl, substituted or non-substituted C₂-C₈-alkynyl, substituted or non-substituted C₁-C₈-alkoxy, substituted or non-substituted C₁-C₈-halogenoalkoxy having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈alkynyloxy, substituted or non-substituted C₃-C₈-halogenoalkynyloxy having 1 to 5 halogen atoms, substituted or non-substituted aryl, substituted or non-substituted aryl-[C₁-C₈]-alkyl;
   as well as salts, N-oxides, metallic complexes and metalloidic complexes thereof

Any of the compounds according to the invention can exist as one or more stereoisomers depending on the number of stereogenic units (as defined by the IUPAC rules) in the compound. The invention thus relates equally to all the stereoisomers, and to the mixtures of all the possible stereoisomers, in all proportions. The stereoisomers can be separated according to the methods which are known per se by the man ordinary skilled in the art.

Notably, the stereostructure of the oxime moiety present in the tetrazolyloxime derivative of formula (I) includes (E) or (Z) isomer, and these stereoisomers form part of the present invention.

According to the invention, the following generic terms are generally used with the following meanings:
halogen means fluorine, chlorine, bromine or iodine;
heteroatom can be nitrogen, oxygen or sulphur;
unless indicated otherwise, a group or a substituent that is substituted according to the invention can be substituted by one or more of the following groups or atoms: a halogen atom, a nitro group, a hydroxy group, a cyano group, an amino group, a sulphenyl group, a pentafluoro-λ⁶-sulphenyl group, a formyl group, a substituted or non-substituted carbaldehyde O—(C₁-C₈-alkyl)oxime, a formyloxy group, a formylamino group, a carbamoyl group, a N-hydroxycarbamoyl group, a formylamino group, a (hydroxyimino)-C₁-C₆-alkyl group, a C₁-C₈-alkyl, a tri(C₁-C₈-alkyl)silyl-C₁-C₈-alkyl, C₃-C₈-cycloalkyl, tri(C₁-C₈alkyl)silyl-C₃-C₈-cycloalkyl, a C₁-C₈-halogenoalkyl having 1 to 5 halogen atoms, a C₃-C₈-halogenocycloalkyl having 1 to 5 halogen atoms, a C₂-C₈-alkenyl, a C₂-C₈-alkynyl, a C₂-C₈-alkenyloxy, a C₂-C₈-alkynyloxy, a C₁-C₈-alkylamino, a di-C₁-C₈-alkylamino, a C₁-C₈-alkoxy, a C₁-C₈-halogenoalkoxy having 1 to 5 halogen atoms, C₃-C₈-cycloalkoxy, a C₁-C₈-alkylsulphenyl, a C₁-C₈-halogenoalkylsulphenyl having 1 to 5 halogen atoms, a C₂-C₈-alkenyloxy, a C₂-C₈-halogenoalkenyloxy having 1 to 5 halogen atoms, a C₃-C₈-alkynyloxy, a C₃-C₈-halogenoalkynyloxy having 1 to 5 halogen atoms, a C₁-C₈-alkylcarbonyl, a C₁-C₈-halogenoalkylcarbonyl having 1 to 5 halogen atoms, a C₁-C₈-alkylcarbamoyl, a di-C₁-C₈-alkylcarbamoyl, a N—C₁-C₈-alkyloxycarbamoyl, a C₁-C₈-alkoxycarbamoyl, a N—C₁-C₈-alkyl-C₁-C₈-alkoxycarbamoyl, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylcarbonylamino, a C₁-C₈-halogenoalkylcarbonylamino having 1 to 5 halogen atoms, a C₁-C₈-alkylaminocarbonyloxy, a di-C₁-C₈-alkylaminocarbonyloxy, a C₁-C₈-alkyloxycarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkylsulphonyl, a C₁-C₈-halogenoalkylsulphonyl having 1 to 5 halogen atoms, a C₁-C₈-alkylamino sulfamoyl, a di-C₁-C₈-alkylaminosulfamoyl, a (C₁-C₆-alkoxyimino)-C₁-C₆-alkyl, a (C₁-C₆-alkenyloxyimino)-C₁-C₆-alkyl, a (C₁-C₆-alkynyloxyimino)-C₁-C₆-alkyl, (benzyloxyimino)-C₁-C₆-alkyl, C₁-C₈-alkoxyalkyl, C₁-C₈-halogenoalkoxyalkyl having 1 to 5 halogen atoms, benzyloxy, benzylsulphenyl, benzylamino, phenoxy, phenylsulphenyl, or phenylamino.
The term “aryl” means phenyl or naphthyl.
The term “heterocyclyl” means saturated or unsaturated 4-, 5-, 6- or 7-membered heterocyclyl comprising up to 4 heteroatoms selected in the list consisting of N, O, S

As a further aspect, the present invention provides hydroximoyl-tetrazole derivatives of formula (Ia), (Ib), (Ic) and (Id)

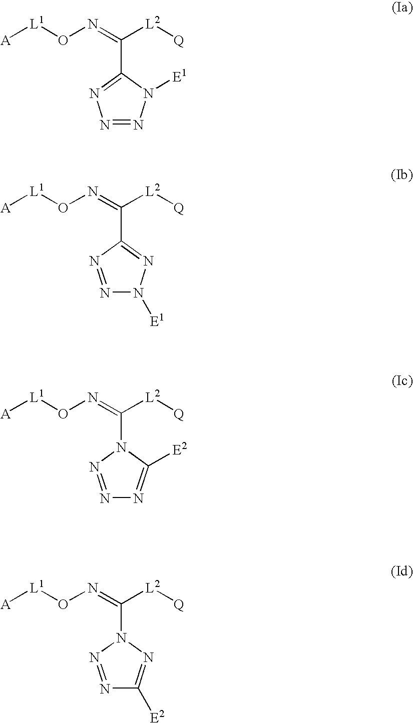

wherein
A, Q, L¹ and L² are defined in the same manner as the corresponding substituents of the compounds of formula (I) according to the invention;
E¹ represents a hydrogen atom, a formyl group, a substituted or non-substituted carbaldehyde O—(C₁-C₈alkyl)oxime, a carbamoyl group, a N-hydroxycarbamoyl group, a substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C₃-C₈-cycloalkyl, substituted or non-substituted tri(C₁-C₈-cycloalkyl, substituted or non-substituted C₁-C₈-halogenoalkyl having 1 to 5 halogen atoms, substituted or non-substituted C₃-C₈-halogenocycloalkyl having 1 to 5 halogen atoms, a C₂-C₈-alkenyl, substituted or non-substituted C₂-C₈-alkynyl, substituted or non-substituted C₁-C₈-alkylcarbonyl, substituted or non-substituted N—(C₁-C₈-alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbamothioyl, substituted or non-substituted di-C₁-C₈-alkylcarbamothioyl, substituted or non-substituted N—C₁-C₈-alkyloxycarbamothioyl, substituted or non-substituted C₁-C₈-alkoxycarbamothioyl, substituted or non-substituted N—C₁-C₈-alkyl-C₁-C₈-alkoxycarbamothioyl, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted C₁-C₈-alkylaminosulfamoyl, substituted or non-substituted di-C₁-C₈-alkylaminosulfamoyl, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, substituted or non-substituted (benzyloxyimino)-C₁-C₆-alkyl, substituted or non-substituted aryl, substituted or non-substituted aryl-[C₁-C₈]-alkyl, substituted or non-substituted tri(C₁-C₈-alkyl)-silyl;
E² represents a hydrogen atom, a halogen atom, a nitro group, a hydroxy group, a cyano group, an amino group, a sulphenyl group, a formyl group, a substituted or non-substituted carbaldehyde O—(C₁-C₈-alkyl)oxime, a formyloxy group, a formylamino group, a carbamoyl group, a N-hydroxycarbamoyl group, a pentafluoro-λ⁶-sulphenyl group, a formylamino group, substituted or non-substituted C₁-C₈-alkoxyamino group, substituted or non-substituted N—C₁-C₈-alkyl-(C₁-C₈-alkoxy)-amino group, substituted or non-substituted (C₁-C₈-alkylamino)-amino group, substituted or non-substituted N—C₁-C₈-alkyl-(C₁-C₈-alkylamino)-amino group, a substituted or non-substituted (hydroxyimino)-C₁-C₈-alkyl group, substituted or non-substituted C₁-C₈alkyl, substituted or non-substituted tri(C₁-C₈alkyl)silyl-C₁-C₈-alkyl, substituted or non-substituted C₃-C₈-cycloalkyl, substituted or non-substituted tri(C₁-C₈-alkyl)silyl-C₃-C₈-cycloalkyl, substituted or non-substituted C₁-C₈-halogenoalkyl having 1 to 5 halogen atoms, substituted or non-substituted C₃-C₈-halogenocycloalkyl having 1 to 5 halogen atoms, a C₂-C₈-alkenyl, substituted or non-substituted C₂-C₈-alkynyl, substituted or non-substituted C₁-C₈alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms, substituted or non-substituted C₁-C₈-alkylsulphenyl, substituted or non-substituted C₁-C₈-halogenoalkylsulphenyl having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms, substituted or non-substituted C₁-C₈-alkylcarbonyl, substituted or non-substituted N—(C₁-C₈-alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈-alkylcarbamoylamino, substituted or non-substituted C₁-C₈-halogenoalkylcarbamoylamino having 1 to 5 halogen atoms, substituted or non-substituted di-C₁-C₈-alkylcarbamoylamino, substituted or non-substituted di-C₁-C₈-halogenoalkylcarbamoylamino having 1 to 5 halogen atoms, substituted or non-substituted N—C₁-C₈-alkyl-(C₁-C₈-alkylcarbamoyl)amino, substituted or non-substituted N—C₁-C₈-alkyl-(C₁-C₈-halogenoalkylcarbamoyl)amino having 1 to 5 halogen atoms, substituted or non-substituted N—C₁-C₈-alkyl-(di-C₁-C₈-alkylcarbamoyl)amino, substituted or non-substituted N—C₁-C₈-alkyl-(di-C₁-C₈-halogenoalkylcarbamoyl)amino having 1 to 5 halogen atoms, substituted or non-substituted C₁-C₈-alkylaminocarbonyloxy, substituted or non-substituted di-C₁-C₈-alkylaminocarbonyloxy, substituted or non-substituted C₁-C₈-alkylcarbamothioyl, substituted or non-substituted di-C₁-C₈-alkylcarbamothioyl, substituted or non-substituted N—C₁-C₈-alkyloxycarbamothioyll, substituted or non-substituted C₁-C₈-alkoxycarbamothioyl, substituted or non-substituted N—C₁-C₈-alkyl-C₁-C₈-alkoxycarbamothioyll, substituted or non-substituted C₁-C₈-alkylthioylamino, substituted or non-substituted C₁-C₈-halogenoalkylthioylamino having 1 to 5 halogen atoms, substituted or non-substituted (C₁-C₈-alkyl-carbamothioyl)-oxy, substituted or non-substituted substituted or non-substituted (di-C₁-C₈-alkyl-carbamothioyl)-oxy, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted C₁-C₈-alkylaminosulfamoyl, substituted or non-substituted di-C₁-C₈-alkylamino sulfamoyl, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, substituted or non-substituted (benzyloxyimino)-C₁-C₆-alkyl, substituted or non-substituted benzyloxy, substituted or non-substituted benzylsulphenyl, substituted or non-substituted benzylamino, substituted or non-substituted phenoxy, substituted or non-substituted phenylsulphenyl, substituted or non-substituted phenylamino, substituted or non-substituted aryl, substituted or non-substituted aryl-[C₁-C₈]-alkyl, substituted or non-substituted tri(C₁-C₈-alkyl)-silyloxy, substituted or non-substituted C₁-C₈-alkylsulfenylamino, substituted or non-substituted C₁-C₈-halogenoalkylsulphinylamino having 1 to 5 halogen atoms, substituted or non-substituted C₁-C₈-alkylsulphonylamino, substituted or non-substituted C₁-C₈-halogenoalkylsulphonylamino having 1 to 5 halogen atoms, substituted or non-substituted C₁-C₈alkoxysulphonylamino, substituted or non-substituted C1-C₈-halogenoxysulphonylamino having 1 to 5 halogen atoms, substituted or non-substituted tri(C₁-C₈alkyl)-silyl, substituted or non-substituted (C₁-C₆-alkylideneamino)oxy, substituted or non-substituted (C₁-C₆-alkenylideneamino)oxy, substituted or non-substituted (C₁-C₆-alkynylideneamino)oxy, substituted or non-substituted (benzylideneamino)oxy.

Preferred compounds of formula (I) and (Ia) to (Id) according to the invention are those wherein L¹ represents a direct bond or a divalent group selected in the list consisting of
—(CR¹R²)ₙ— —C(═O)—(CR¹R²)ₚ—
—(CR¹R²)ₘ—O— —(CR¹R²)ₘ—C(═O)—O—
—(CR¹R²)ₘ—NH— —(CR¹R²)ₘ—C(═O)—NH—
—(CR¹R²)ₘ—C(═O)— —(CR¹R²)ₘ—NH—C(═O)
   wherein
n represents 1 or 2;
m and p independently represent 0 or 1;
R¹ and R² independently represent a hydrogen atom, a halogen atom, a cyano group, substituted or non-substituted C₁-C₈-alkyl, substituted or non-substituted C₃-C₈-cycloalkyl, substituted or non-substituted C₁-C₈-halogenoalkyl having 1 to 5 halogen atoms, substituted or non-substituted C₃-C₈-halogenocycloalkyl having 1 to 5 halogen atoms, a C₂-C₈-alkenyl, substituted or non-substituted C₂-C₈-alkynyl, substituted or non-substituted C₁-C₈-alkoxy, substituted or non-substituted C₁-C₈-halogenoalkoxy having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms.

More preferred compounds of formula (I) and (Ia) to (Id) according to the invention are those wherein L¹ represents a direct bond or a divalent group selected in the list consisting of —(CR¹R²)—, —C(═O)—(CR¹R²)— and —C(═O)—; wherein R¹ and R² are independently selected in the list consisting of hydrogen, halogen, methyl, ethyl, isopropyl, trifluoromethyl, difluoromethyl, allyl, ethynyl, propargyl, cyclopropyl, methoxy, trifluoromethoxy and cyano.

Other preferred compounds of formula (I) and (Ia) to (Id) according to the invention are those wherein L² represents a direct bond or a divalent group selected in the list consisting of
—(CR³R⁴)_{q}— —(CR³R⁴)ₐ—C(═O)—
—(CR³═CR⁴)— —(CR³R⁴)ₐ—C(═O)—O—
—C≡C— —(CR³R⁴)ₐ—O—C(═O)—
—(CR³R⁴)ₐ—O— —(CR³R⁴)ₐ—C(═O)—NH—
—(CR³R⁴)ₐ—NH— —(CR³R⁴)ₐ—NH—C(═O)—
   wherein
q and a independently represent 1 or 2;
R³ and R⁴ independently represent a hydrogen atom, a halogen atom, a cyano group, substituted or non-substituted C₁-C₈-alkyl, substituted or non-substituted C₃-C₈-cycloalkyl, substituted or non-substituted C₁-C₈-halogenoalkyl having 1 to 5 halogen atoms, substituted or non-substituted C₃-C₈-halogenocycloalkyl having 1 to 5 halogen atoms, a C₂-C₈-alkenyl, substituted or non-substituted C₂-C₈-alkynyl, substituted or non-substituted C₁-C₈-alkoxy, substituted or non-substituted C₁-C₈-halogenoalkoxy having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms.

Other more preferred compounds of formula (I) and (Ia) to (Id) according to the invention are those wherein L² represents a direct bond or —(CR³R⁴)— wherein R³ and R⁴ are independently selected in the list consisting of hydrogen, halogen, methyl, ethyl, isopropyl, trifluoromethyl, difluoromethyl, allyl, ethynyl, propargyl, cyclopropyl, methoxy, trifluoromethoxy and cyano.

Still other preferred compounds of formula (I) and (Ia) to (Id) according to the invention are those wherein A is selected in the list consisting of A¹ to A⁵⁸.

Other more preferred compounds of formula (I) and (Ia) to (Id) according to the invention are those wherein A is selected in the list consisting of A², A⁶, A⁸, A¹¹, A¹², A¹³, A¹⁴, A¹⁵, A¹⁶, A¹⁷ and A¹⁸.

Other more preferred compounds of formula (I) and (Ia) to (Id) according to the invention are those wherein Z¹ represents a hydrogen atom, a halogen atom, a hydroxy group, a cyano group, an amino group, a formyloxy group, a formylamino group, a carbamoyl group, a N-hydroxycarbamoyl group, a pentafluoro-λ⁶-sulphenyl group, a formylamino group, substituted or non-substituted C₁-C₈-alkoxyamino group, substituted or non-substituted N—C₁-C₈-alkyl-(C₁-C₈-alkoxy)-amino group, a substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C₃-C₈-cycloalkyl, substituted or non-substituted tri(C₁-C₈-alkyl)silyl-C₃-C₈-cycloalkyl, substituted non-substituted C₁-C₈-halogenoalkyl having 1 to 5 halogen atoms, substituted or non-substituted C₃-C₈-halogenocycloalkyl having 1 to 5 halogen atoms, a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted (C₁-C₈-alkoxycarbonyl)amino, substituted or non-substituted (C₂-C₈-alkenyloxycarbonyl)amino, substituted or non-substituted (C₃-C₈-alkynyloxycarbonyl)amino, substituted or non-substituted (C₃-C₈-cycloalkoxycarbonyl)amino, substituted or non-substituted C₁-C₈-halogenoalkoxy having 1 to 5 halogen atoms, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkylcarbonyl, substituted or non-substituted N—(C₁-C₈-alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonyl having 1 to 5 halogen atoms, substituted or non-substituted C₁-C₈-alkylcarbamoylamino, substituted or non-substituted C₁-C₈-halogenoalkylcarbamoylamino having 1 to 5 halogen atoms, substituted or non-substituted di-C₁-C₈-alkylcarbamoylamino, substituted or non-substituted di-C₁-C₈-halogenoalkylcarbamoylamino having 1 to 5 halogen atoms, substituted or non-substituted N—C₁-C₈-alkyl-(C₁-C₈-alkylcarbamoyl)amino, substituted or non-substituted N—C₁-C₈-alkyl-(C₁-C₈-halogenoalkylcarbamoyl)amino having 1 to 5 halogen atoms, substituted or non-substituted N—C₁-C₈-alkyl-(di-C₁-C₈-alkylcarbamoyl)amino, substituted or non-substituted N—C₁-C₈-alkyl-(di-C₁-C₈-halogenoalkylcarbamoyl)amino having 1 to 5 halogen atoms, substituted or non-substituted C₁-C₈-alkylaminocarbonyloxy, substituted or non-substituted di-C₁-C₈-alkylaminocarbonyloxy, substituted or non-substituted C₁-C₈-alkylcarbamothioyl, substituted or non-substituted di-C₁-C₈-alkylcarbamothioyl, substituted or non-substituted N—C₁-C₈-alkyloxycarbamothioyl, substituted or non-substituted C₁-C₈-alkoxycarbamothioyl, substituted or non-substituted N—C₁-C₈-alkyl-C₁-C₈-alkoxycarbamothioyl, substituted or non-substituted C₁-C₈-alkylthioylamino, substituted or non-substituted C₁-C₈-halogenoalkylthioylamino having 1 to 5 halogen atoms, substituted or non-substituted (C₁-C₈-alkyl-carbamothioyl)-oxy, substituted or non-substituted substituted or non-substituted (di-C₁-C₈-alkyl-carbamothioyl)-oxy, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkylaminosulfamoyl, substituted or non-substituted di-C₁-C₈-alkylaminosulfamoyl, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, substituted or non-substituted (benzyloxyimino)-C₁-C₆-alkyl, substituted or non-substituted benzyloxy, substituted or non-substituted benzylsulphenyl, substituted or non-substituted benzylamino, substituted or non-substituted phenoxy, substituted or non-substituted phenylsulphenyl, substituted or non-substituted phenylamino, substituted or non-substituted aryl, substituted or non-substituted aryl-[C₁-C₈]-alkyl, substituted or non-substituted tri(C₁-C₈-alkyl)-silyloxy, substituted or non-substituted C₁-C₈-halogenoalkylsulphinylamino having 1 to 5 halogen atoms, substituted or non-substituted C₁-C₈alkylsulphonylamino, substituted or non-substituted C₁-C₈-halogenoalkylsulphonylamino having 1 to 5 halogen atoms, substituted or non-substituted C₁-C₈-alkoxysulphonylamino, substituted or non-substituted C₁-C₈-halogenoxysulphonylamino having 1 to 5 halogen atoms, substituted or non-substituted tri(C₁-C₈-alkyl)-silyl, substituted or non-substituted (C₁-C₈-alkylideneamino)oxy, substituted or non-substituted (C₁-C₆-alkenylideneamino)oxy, substituted or non-substituted (C₁-C₈-alkynylideneamino)oxy, substituted or non-substituted (benzylideneamino)oxy.

Other even more preferred compounds of formula (I) and (Ia) to (Id) according to the invention are those wherein Z¹ represents a hydrogen atom, a halogen atom, a cyano group, an amino group, a formyloxy group, a formylamino group, a carbamoyl group, a N-hydroxycarbamoyl group, a formylamino group, substituted or non-substituted C₁-C₈alkoxyamino group, substituted or non-substituted N—C₁-C₈-alkyl-(C₁-C₈-alkoxy)-amino group, substituted or non-substituted C₁-C₈-alkyl, substituted or non-substituted tri(C₁-C₈-alkyl)silyl-C₁-C₈-alkyl, substituted or non-substituted C₃-C₈-cycloalkyl, substituted or non-substituted C₁-C₈-halogenoalkyl having 1 to 5 halogen atoms, a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted (C₁-C₈-alkoxycarbonyl)amino, substituted or non-substituted (C₂-C₈-alkenyloxycarbonyl)amino, substituted or non-substituted (C₃-C₈-alkynyloxycarbonyl)amino, substituted or non-substituted (C₃-C₈-cycloalkoxycarbonyl)amino, substituted or non-substituted C₁-C₈-halogenoalkoxy having 1 to 5 halogen atoms, substituted or non-substituted C₁-C₈-alkylsulphenyl, substituted or non-substituted C₁-C₈-alkylcarbonyl, substituted or non-substituted N—(C₁-C₈-alkoxy)-C₁-C₈-alkanimidoyl, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈-alkylcarbamoylamino, substituted or non-substituted C₁-C₈-halogenoalkylcarbamoylamino having 1 to 5 halogen atoms, substituted or non-substituted di-C₁-C₈-alkylcarbamoylamino, substituted or non-substituted di-C₁-C₈-halogenoalkylcarbamoylamino having 1 to 5 halogen atoms, substituted or non-substituted N—C₁-C₈-alkyl-(C₁-C₈-alkylcarbamoyl)amino, substituted or non-substituted N—C₁-C₈-alkyl-(C₁-C₈-halogenoalkylcarbamoyl)amino having 1 to 5 halogen atoms, substituted or non-substituted N—C₁-C₈-alkyl-(di-C₁-C₈-alkylcarbamoyl)amino, substituted or non-substituted N—C₁-C₈alkyl-(di-C₁-C₈-halogenoalkylcarbamoyl)amino having 1 to 5 halogen atoms, substituted or non-substituted C₁-C₈-alkylcarbamothioyl, substituted or non-substituted di-C₁-C₈-alkylcarbamothioyl, substituted or non-substituted N—C₁-C₈-alkyloxycarbamothioyl, substituted or non-substituted C₁-C₈-alkoxycarbamothioyl, substituted or non-substituted N—C₁-C₈-alkyl-C₁-C₈-alkoxycarbamothioyl, substituted or non-substituted C₁-C₈-alkylthioylamino, substituted or non-substituted C₁-C₈-halogenoalkylthioylamino having 1 to 5 halogen atoms, substituted or non-substituted benzylamino, substituted or non-substituted phenoxy, substituted or non-substituted phenylamino, substituted or non-substituted aryl, substituted or non-substituted aryl-[C₁-C₈]-alkyl, substituted or non-substituted C₁-C₈-halogenoalkylsulphinylamino having 1 to 5 halogen atoms, substituted or non-substituted C₁-C₈-alkylsulphonylamino, substituted or non-substituted C₁-C₈-halogenoalkylsulphonylamino having 1 to 5 halogen atoms.

Still other preferred compounds of formula (I) and (Ia) to (Id) according to the invention are those wherein Z², Z³, Z⁴, Z⁵, Z⁶, Z⁷, Z⁸ and Z⁹ independently represent a hydrogen atom, a halogen atom, a cyano group, substituted or non-substituted C₁-C₈-alkyl, substituted or non-substituted C₃-C₈-cycloalkyl, substituted or non-substituted C₁-C₈-halogenoalkyl having 1 to 5 halogen atoms, a C₂-C₈-alkenyl, substituted or non-substituted C₂-C₈-alkynyl, substituted or non-substituted C₁-C₈-alkoxy, substituted or non-substituted C₁-C₈-halogenoalkoxy having 1 to 5 halogen atoms, substituted or non-substituted phenoxy, substituted or non-substituted aryl, substituted or non-substituted aryl-[C₁-C₈]-alkyl.

Other more preferred compounds of formula (I) and (Ia) to (Id) according to the invention are those wherein Z², Z³, Z⁴, Z⁵, Z⁶, Z⁷, Z⁸ and Z⁹ are independently selected in the list consisting of hydrogen, halogen, methyl, ethyl, isopropyl, isobutyl, terbutyl, trifluoromethyl, difluoromethyl, allyl, ethynyl, propargyl, cyclopropyl, methoxy, trifluoromethoxy, acetyl, and cyano.

Still other preferred compounds of formula (I) and (Ia) to (Id) according to the invention are those wherein K¹ and K² are independently selected in the list consisting of hydrogen, methyl, ethyl, isopropyl, isobutyl, terbutyl, allyl, propargyl, cyclopropyl, acetyl, trifluoroacetyl and mesyl.

Still other preferred compounds of formula (I) and (Ia) to (Id) according to the invention are those wherein X¹ independently represents a cyano group, an amino group, a sulphenyl group, a pentafluoro-λ⁶-sulphenyl group, substituted or non-substituted C₁-C₈-alkyl, substituted or non-substituted tri(C₁-C₈alkyl)silyl-C₁-C₈-alkyl, substituted or non-substituted C₃-C₈-cycloalkyl, substituted or non-substituted tri(C₁-C₈-alkyl)silyl-C₃-C₈-cycloalkyl, substituted or non-substituted C₁-C₈-halogenoalkyl having 1 to 5 halogen atoms, substituted or non-substituted C₃-C₈-halogenocycloalkyl having 1 to 5 halogen atoms, a C₂-C₈-alkenyl, substituted or non-substituted C₂-C₈-alkynyl, substituted or non-substituted C₁-C₈-alkoxy, substituted or non-substituted C₁-C₈-halogenoalkoxy having 1 to 5 halogen atoms, substituted or non-substituted C₁-C₈-alkylsulphenyl, substituted or non-substituted C₁-C₈-halogenoalkylsulphenyl having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms, substituted or non-substituted C₁-C₈alkylcarbonyl, substituted or non-substituted N—(C₁-C₈-alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbamothioyl, substituted or non-substituted di-C₁-C₈alkylcarbamothioyl, substituted or non-substituted N—C₁-C₈alkyloxycarbamothioyl, substituted or non-substituted C₁-C₈-alkoxycarbamothioyl, substituted or non-substituted N—C₁-C₈-alkyl-C₁-C₈-alkoxycarbamothioyl, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, substituted or non-substituted (benzyloxyimino)-C₁-C₆-alkyl, substituted or non-substituted benzyloxy, substituted or non-substituted benzylsulphenyl, substituted or non-substituted phenoxy, substituted or non-substituted phenylsulphenyl, substituted or non-substituted aryl, substituted or non-substituted aryl-[C₁-C₈]-alkyl, substituted or non-substituted tri(C₁-C₈-alkyl)-silyloxy, substituted or non-substituted tri(C₁-C₈-alkyl)-silyl;

Other more preferred compounds of formula (I) and (Ia) to (Id) according to the invention are those wherein X¹ independently represents a cyano group, an amino group, a sulphenyl group, a pentafluoro-λ⁶-sulphenyl group, substituted or non-substituted C₁-C₈-alkyl, substituted or non-substituted tri(C₁-C₈-alkyl)silyl-C₁-C₈-alkyl, substituted or non-substituted C₃-C₈-cycloalkyl, substituted or non-substituted tri(C₁-C₈-alkyl)silyl-C₃-C₈-cycloalkyl, substituted or non-substituted C₁-C₈-halogenoalkyl having 1 to 5 halogen atoms, substituted or non-substituted C₃-C₈-halogenocycloalkyl having 1 to 5 halogen atoms, a C₂-C₈-alkenyl, substituted or non-substituted C₂-C₈-alkynyl, substituted or non-substituted C₁-C₈-alkoxy, substituted or non-substituted C₁-C₈-halogenoalkoxy having 1 to 5 halogen atoms, substituted or non-substituted C₁-C₈-alkylsulphenyl, substituted or non-substituted C₁-C₈-halogenoalkylsulphenyl having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms, substituted or non-substituted C₁-C₈-alkylcarbonyl, substituted or non-substituted N—(C₁-C₈-alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbamothioyl, substituted or non-substituted di-C₁-C₈-alkylcarbamothioyl, substituted or non-substituted N—C₁-C₈-alkyloxycarbamothioyll, substituted or non-substituted C₁-C₈-alkoxycarbamothioyl, substituted or non-substituted N—C₁-C₈-alkyl-C₁-C₈-alkoxycarbamothioyl, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, substituted or non-substituted (benzyloxyimino)-C₁-C₈-alkyl, substituted or non-substituted benzyloxy, substituted or non-substituted benzylsulphenyl, substituted or non-substituted phenoxy, substituted or non-substituted phenylsulphenyl, substituted or non-substituted aryl, substituted or non-substituted aryl-[C₁-C₈]-alkyl, substituted or non-substituted tri(C₁-C₈-alkyl)-silyloxy, substituted or non-substituted tri(C₁-C₈-alkyl)-silyl.

Other more preferred compounds of formula (I) and (Ia) to (Id) according to the invention are those wherein X¹ independently represents methyl, isopropyl, isobutyl, tertbutyl, trifluoromethyl, difluoromethyl, allyl, ethynyl, propargyl, cyclopropyl, benzyl, phenethyl, methoxy, trifluoromethoxy, acetyl, trifluoroacetyl and cyano.

Other more preferred compounds of formula (I) and (Ia) to (Id) according to the invention are those wherein X² independently represents a halogen atom, a cyano group, an amino group, a sulphenyl group, a pentafluoro-λ⁶-sulphenyl group, substituted or non-substituted C₁-C₈-alkyl, substituted or non-substituted tri(C₁-C₈-alkyl)silyl-C₁-C₈-alkyl, substituted or non-substituted C₃-C₈-cycloalkyl, substituted or non-substituted tri(C₁-C₈-alkyl)silyl-C₃-C₈-cycloalkyl, substituted or non-substituted C₁-C₈-halogenoalkyl having 1 to 5 halogen atoms, substituted or non-substituted C₃-C₈-halogenocycloalkyl having 1 to 5 halogen atoms, a C₂-C₈-alkenyl, substituted or non-substituted C₂-C₈-alkynyl, substituted or non-substituted C₁-C₈-alkoxy, substituted or non-substituted C₁-C₈-halogenoalkoxy having 1 to 5 halogen atoms, substituted or non-substituted C₁-C₈-alkylsulphenyl, substituted or non-substituted C₁-C₈-halogenoalkylsulphenyl having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈alkynyloxy, substituted or non-substituted C₃-C₈-halogenoalkynyloxy having 1 to 5 halogen atoms, substituted or non-substituted C₁-C₈-alkylcarbonyl, substituted or non-substituted N—(C₁-C₈-alkoxy)-C₁-C₈alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbamothioyl, substituted or non-substituted di-C₁-C₈alkylcarbamothioyl, substituted or non-substituted N—C₁-C₈-alkyloxycarbamothioyll, substituted or non-substituted C₁-C₈alkoxycarbamothioyl, substituted or non-substituted N—C₁-C₈-alkyl-C₁-C₈-alkoxycarbamothioyl, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, substituted or non-substituted (benzyloxyimino)-C₁-C₆-alkyl, substituted or non-substituted benzyloxy, substituted or non-substituted benzylsulphenyl, substituted or non-substituted phenoxy, substituted or non-substituted phenylsulphenyl, substituted or non-substituted aryl, substituted or non-substituted aryl-[C₁-C₁]-alkyl, substituted or non-substituted tri(C₁-C₈-alkyl)-silyloxy, substituted or non-substituted tri(C₁-C₈alkyl)-silyl.

Other more preferred compounds of formula (I) and (Ia) to (Id) according to the invention are those wherein X² independently represents a halogen atom, methyl, isopropyl, isobutyl, tertbutyl, trifluoromethyl, difluoromethyl, allyl, ethynyl, propargyl, cyclopropyl, benzyl, phenethyl, methoxy, trifluoromethoxy, acetyl, trifluoroacetyl and cyano.

Preferred compounds of formula (Ia) to (Id) according to the invention are those wherein E¹ represents a hydrogen atom, a formyl group, a substituted or non-substituted carbaldehyde O—(C₁-C₈-alkyl)oxime, a substituted or non-substituted C₁-C₈-alkyl, substituted or non-substituted tri(C₁-C₈-alkyl)silyl-C₁-C₈-alkyl, substituted or non-substituted C3-C8-cycloalkyl, substituted or non-substituted C₂-C₈-alkynyl, substituted or non-substituted C₁-C₈-alkylcarbonyl, substituted or non-substituted N—(C₁-C₈-alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted C₁-C₈-alkylaminosulfamoyl, substituted or non-substituted di-C₁-C₈-alkylaminosulfamoyl.

More preferred compounds of formula (Ia) to (Id) according to the invention are those wherein E¹ represents a formyl group, methyl, ethyl, iso-propyl, allyl, propargyl, cyclopropyl, substituted or non-substituted C₁-C₈alkylcarbonyl.

Other preferred compounds of formula (Ia) to (Id) according to the invention are those wherein E² represents a hydrogen atom, a halogen atom, a formyl group, a substituted or non-substituted carbaldehyde O—(C₁-C₈-alkyl)oxime, substituted or non-substituted C₁-C₈-alkyl, substituted or non-substituted C₃-C₈-cycloalkyl, substituted or non-substituted C₁-C₈-halogenoalkyl having 1 to 5 halogen atoms, a C₂-C₈-alkenyl, substituted or non-substituted C₂-C₈-alkynyl, substituted or non-substituted C₁-C₈-alkoxy, substituted or non-substituted C₁-C₈-halogenoalkoxy having 1 to 5 halogen atoms, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈-alkylsulphenyl, substituted or non-substituted benzyloxy, substituted or non-substituted benzylsulphenyl, substituted or non-substituted phenoxy, substituted or non-substituted phenylsulphenyl, substituted or non-substituted aryl, substituted or non-substituted aryl-[C₁-C₈]-alkyl, substituted or non-substituted tri(C₁-C₈-alkyl)-silyl, substituted or non-substituted (C₁-C₆-alkylideneamino)oxy, substituted or non-substituted (C₁-C₆-alkenylideneamino)oxy, substituted or non-substituted (C₁-C₆-alkynylideneamino)oxy, substituted or non-substituted (benzylideneamino)oxy.

Other more preferred compounds of formula (Ia) to (Id) according to the invention are those wherein E² represents a halogen atom, methyl, ethyl, isopropyl, trifluoromethyl, difluoromethyl, allyl, ethynyl, propargyl, cyclopropyl, cyano.

The above mentioned preferences with regard to the substituents of the compounds of formula (I) and (Ia) to (Id) according to the invention can be combined in various manners. These combinations of preferred features thus provide sub-classes of compounds according to the invention. Examples of such sub-classes of preferred compounds according to the invention can combine:
preferred features of A with preferred features of one or more of L¹, L², Q, E¹ and E²;
preferred features of L¹ with preferred features of one or more of A, L², Q, E¹ and E²;
preferred features of L² with preferred features of one or more of A, L¹, Q, E¹ and E²;
preferred features of Q with preferred features of one or more of A, L¹, L², E¹ and E²;
preferred features of E¹ with preferred features of one or more of A, L¹, L², Q and E²;
preferred features of E² with preferred features of one or more of A, L¹, L², Q and E¹.

In these combinations of preferred features of the substituents of the compounds according to the invention, the said preferred features can also be selected among the more preferred features of each of A, Q, L¹, L², E¹ and E²; so as to form most preferred subclasses of compounds according to the invention.

The preferred features of the other substituents of the compounds according to the invention can also be part of such sub-classes of preferred compounds according to the invention, notably the groups of substituents R, Z, K, G, X¹ and X² as well as the integers a, b, m, n, p, q, r and t.

The present invention also relates to a process for the preparation of compounds of formula (I), (Ia), (Ib), (Ic) and (Id). Thus, according to a further aspect of the present invention, there is a provided process P1 for the preparation of compounds of formula (I), (Ia), (Ib), (Ic) and (Id) as herein-defined, as illustrated by the following reaction schemes.

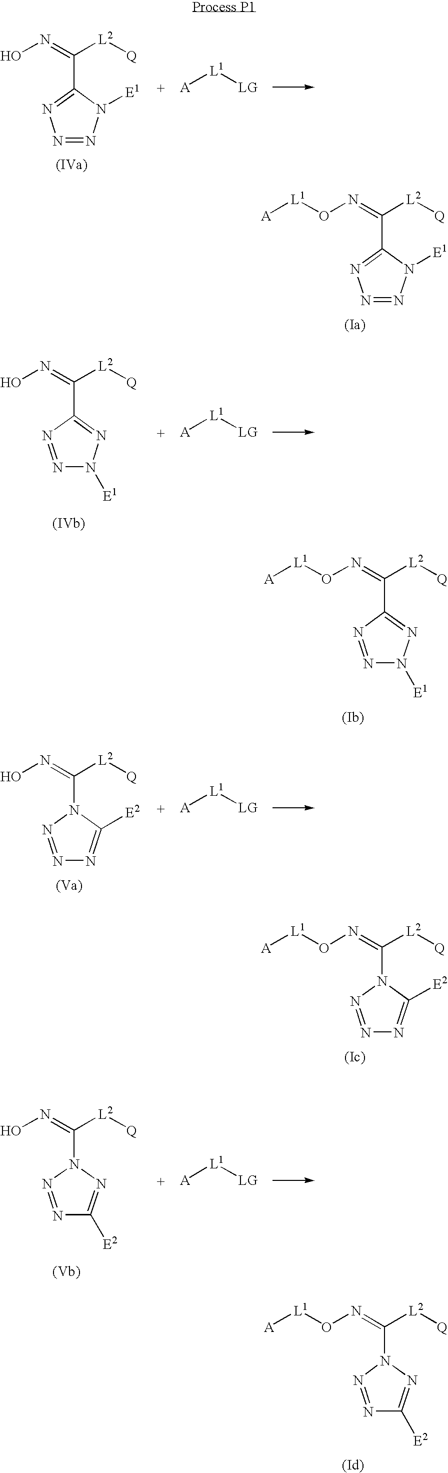

wherein

A, L¹, L², Q, E¹ and E² are as herein-defined and LG represents a leaving group. Suitable leaving groups can be selected in the list consisting of a halogen atom or other customary nucleofugal groups such as triflate, mesylate, or tosylate.

If Z¹, Z², Z³, Z⁴, Z⁵, Z⁶, Z⁷, Z⁸ or Z⁹ represents a protected, substituted or non-substituted C₁-C₈-alkylamino, process P1 according to the invention can be completed by a deprotection step in order to yield the substituted or non-substituted C₁-C₈-alkylamino group. Amino-protecting groups and related methods of cleavage thereof are known and can be found in T. W. Greene and P. G. M. Wuts, Protective Group in Organic Chemistry, —3^{rd} ed., John Wiley & Sons.

For the compounds of formula (I) according to the invention when Z¹, Z², Z³, Z⁴, Z⁵, Z⁶, Z⁷, Z⁸ or Z⁹ represents an amino group, substituted or non-substituted C₁-C₈-alkylamino group, process P1 according to the invention can be completed by a further step comprising the additional modification of this group, notably by a reaction of acylation, alkoxycarbonylation, alkylaminocarbonylation or alkylaminothiocarbonylation, according to known methods. In such a case there is provided a process P2 according to the invention and such a process P2 can be illustrated by the following reaction schemes:

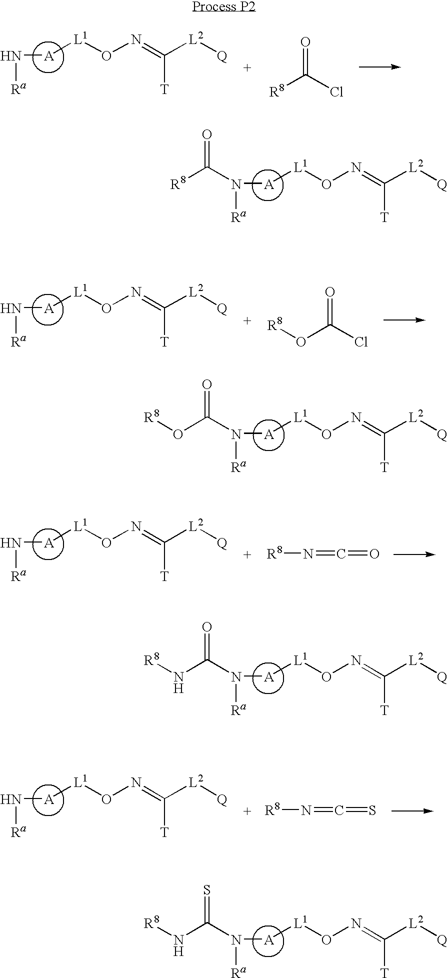

wherein
A, L¹, L², T, Q are as herein-defined;
R^{a} represents a hydrogen atom, substituted or non-substituted C₁-C₈-alkyl;
R⁸ represents a substituted or non-substituted C₁-C₈-alkyl, substituted or non-substituted tri(C₁-C₈-alkyl)silyl-C₁-C₈-alkyl, substituted or non-substituted C₃-C₈-cycloalkyl, substituted or non-substituted tri(C₁-C₈-alkyl)silyl-C₃-C₈-cycloalkyl, substituted or non-substituted C₁-C₈-halogenoalkyl having 1 to 5 halogen atoms, substituted or non-substituted C₃-C₈-halogenocycloalkyl having 1 to 5 halogen atoms, a C₂-C₈-alkenyl, substituted or non-substituted C₂-C₈-alkynyl, substituted or non-substituted C₁-C₈-alkoxy, substituted or non-substituted C₁-C₈-halogenoalkoxy having 1 to 5 halogen atoms, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, substituted or non-substituted (benzyloxyimino)-C₁-C₆-alkyl, substituted or non-substituted benzyloxy, substituted or non-substituted phenoxy, substituted or non-substituted aryl, substituted or non-substituted aryl-[C₁-C₈]-alkyl.

If Z¹, Z², Z³, Z⁴, Z⁵, Z⁶, Z⁷, Z⁸ or Z⁹ represents a protected amino group, carrying out process P2 would previously require a deprotection step in order to yield the amino group. Amino-protecting groups and related methods of cleavage thereof are known and can be found in T. W. Greene and P. G. M. Wuts, Protective Group in Organic Chemistry, 3^{rd} ed., John Wiley & Sons.

For the compounds of formula (I) according to the invention when Z¹, Z², Z³, Z⁴, Z⁵, Z⁶, Z⁷, Z⁸ or Z⁹ represents an amino group, a formylamino group, substituted or non-substituted C₁-C₈-alkylamino, substituted or non-substituted C₁-C₈-alkylcarbonylamino, substituted or non-substituted benzylamino, substituted or non-substituted (C₁-C₈-alkoxycarbonyl)amino, substituted or non-substituted (C₂-C₈-alkenyloxycarbonyl)amino, substituted or non-substituted (C₃-C₈-alkynyloxycarbonyl)amino, substituted or non-substituted (C₃-C₈-cycloalkoxycarbonyl)amino, process P1 according to the invention can be completed by a further step comprising the additional modification of this group, notably by a reaction of alkylation, according to known methods. In such a case there is provided a process P3 according to the invention and such a process P3 can be illustrated by the following reaction scheme:

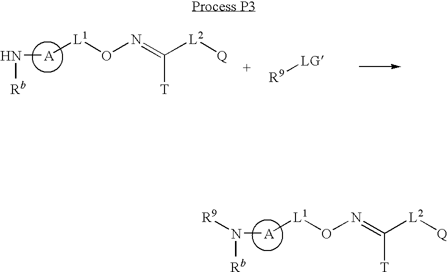

wherein
A, L¹, L², T, Q are as herein-defined;
R^{b} represents a hydrogen atom, a formyl group, substituted or non-substituted C₁-C₈-alkyl, substituted or non-substituted C₁-C₈-alkylcarbonyl, substituted or non-substituted benzyl, substituted or non-substituted C₁-C₈alkoxycarbonyl, substituted or non-substituted C₂-C₈-alkenyloxycarbonyl, substituted or non-substituted C₃-C₈-alkynyloxycarbonyl, substituted or non-substituted C₃-C₈-cycloalkoxycarbonyl;
R⁹ represents substituted or non-substituted C₁-C₈-alkyl,
LG′ represents a leaving group. Suitable leaving groups can be selected in the list consisting of a halogen atom or other customary nucleofugal groups such as triflate, mesylate, or tosylate.

For the compounds of formula (I) according to the invention when Z¹, Z², Z³, Z⁴, Z⁵, Z⁶, Z⁷, Z⁸ or Z⁹ represents a amino group or a substituted or non-substituted C₁-C₈-alkylamino, process P1 according to the invention can be completed by a further step comprising the additional modification of this group, notably by a reaction of amino-reduction, according to known methods. In such a case there is provided a process P4 according to the invention and such a process P4 can be illustrated by the following reaction scheme:

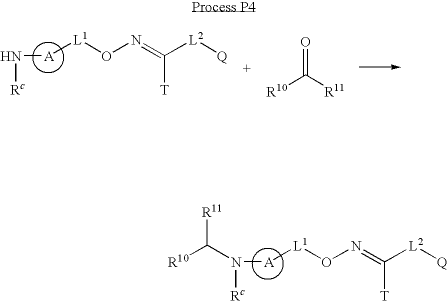

wherein
A, L¹, L², T, Q are as herein-defined;
R^{e} represents a hydrogen atom, substituted or non-substituted C₁-C₈-alkyl;
R¹⁰ represents a substituted or non-substituted C₁-C₈-alkyl and R¹¹ represents a hydrogen atom, a substituted or non-substituted C₁-C₈-alkyl.

For the compounds of formula (Ia) according to the invention when Z¹, Z², Z³, Z⁴, Z⁵, Z⁶, Z⁷, Z⁸ or Z⁹ represents a halogen atom, substituted or non-substituted C₁-C₈-halogenoalkylsulphonyloxy having 1 to 5 halogen atoms, process P1 according to the invention can be completed by a further step comprising the displacement of this group, notably by a reaction of aromatic nucleophilic substitution or organo-metallic coupling, according to known methods, optionally in the presence of a catalyst, preferably a transition metal catalyst, such as a copper salt, preferably a copper(I) salt for example copper(I) chloride, copper(I) cyanide, such as palladium salts or complexes for example palladium (II) chloride, palladium (II) acetate, tetrakis-(triphenylphosphine) palladium(0), bis-(triphenylphosphine) palladium dichloride (II), tris(dibenzylideneacetone) dipalladium(0), bis(dibenzylideneacetone) palladium(0), or 1,1′-bis(diphenylphosphino)ferrocene-palladium (II) chloride. As an alternative the palladium complex is directly generated in the reaction mixture by separately adding to the reaction mixture a palladium salt and a complex ligand such as a phosphine, for example triethylphosphine, tri-tert-butylphosphine, tricyclohexylphosphine, 2-(dicyclohexylphosphine)biphenyl, 2-(di-tert-butylphosphin)biphenyl, 2-(dicyclohexylphosphine)-2′-(N,N-dimethylamino)-biphenyl, triphenylphosphine, tris-(o-tolyl)phosphine, sodium 3-(diphenylphosphino)benzolsulfonate, tris-2-(methoxyphenyl)phosphine, 2,2′-bis-(diphenylphosphine)-1,1′-binaphthyl, 1,4-bis-(diphenylphosphine)butane, 1,2-bis -(diphenylphosphine)ethane, 1,4-bis-(dicyclohexylphosphine)butane, 1,2-bis-(dicyclohexylphosphine)ethane, 2-(dicyclohexylphosphine)-2’-(N,N-dimethylamino)-biphenyl, bis(diphenylphosphino)ferrocene, tris-(2,4-tert-butylphenyl)-phosphite, (R)-(−)-1-[(S)-2-(diphenylphosphino)ferrocenyl]ethyldi-tert-butylphosphine, (S)-(+)-1-[(R)-2-(diphenylphosphino)ferrocenyl]ethyldicyclohexylphosphine, (R)-(−)-1-[(S)-2-(diphenylphosphino)ferrocenyl]ethyldicyclohexylphosphine, (S)-(+)-1-[(R)-2-(diphenylphosphino)ferrocenyl]ethyldi-t-butylphosphine, optionally in the presence of a base, such as an inorganic or an organic base; preferably an alkaline earth metal or alkali metal hydrides, hydroxides, amides, alcoholates, acetates, carbonates or hydrogen carbonates, such as sodium hydride, sodium amide, lithiium diisopropylamide, sodium methanolate, sodium ethanolate, potassium tert-butanolate, sodium acetate, potassium acetate, calcium acetate, sodium hydroxide, potassium hydroxide, sodium carbonate, potassium carbonate, potassium bicarbonate, sodium bicarbonate, cesium carbonate or ammonium carbonate; and also tertiary amines, such as trimethylamine, triethylamine (TEA), tributylamine, N,N-dimethylaniline, N,N-dimethyl-benzylamine, N,N-diisopropyl-ethylamine (DIPEA), pyridine, N-methylpiperidine, N-methylmorpholine, N,N-dimethylaminopyridine, diazabicyclooctane (DABCO), diazabicyclononene (DBN) or diazabicycloundecene (DBU). In such a case there is provided a process P5 according to the invention and such a process P5 can be illustrated by the following reaction scheme:

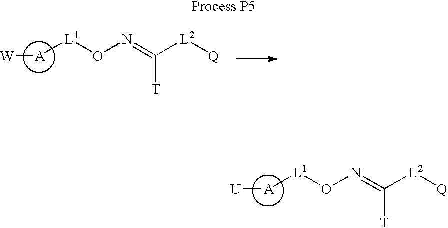

wherein
A, L¹, L², T, Q are as herein-defined;
W represents a halogen atom, substituted or non-substituted C₁-C₈-halogenoalkylsulphonyloxy having 1 to 5 halogen atoms, a C₁-C₆ alkylsulphenyl, a C₁-C₆ haloalkylsulphenyl; a substituted or non-substitued phenylsulphenyl;
U represents a hydroxy group, a cyano group, an amino group, a formyloxy group, a formylamino group, substituted or non-substituted C₁-C₈-alkoxyamino group, substituted or non-substituted N—C₁-C₈-alkyl-(C₁-C₈-alkoxy)-amino group, substituted or non-substituted (C₁-C₈-alkylamino)-amino group, substituted or non-substituted N—C₁-C₈-alkyl-(C₁-C₈-alkylamino)-amino group, substituted or non-substituted C₁-C₈-alkyl, substituted or non-substituted C₃-C₈-cycloalkyl, substituted or non-substituted tri(C₁-C₈-alkyl)silyl-C₃-C₈-cycloalkyl, substituted or non-substituted C₁-C₈-halogenoalkyl having 1 to 5 halogen atoms, substituted or non-substituted C₃-C₈-halogenocycloalkyl having 1 to 5 halogen atoms, a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈alkoxy, substituted or non-substituted C₁-C₈-halogenoalkoxy having 1 to 5 halogen atoms, substituted or non-substituted C₁-C₈-alkylsulphenyl, substituted or non-substituted C₁-C₈-halogenoalkylsulphenyl having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted (C₁-C₈-alkoxycarbonyl)amino, substituted or non-substituted (C₂-C₈-alkenyloxycarbonyl)amino, substituted or non-substituted (C₃-C₈-alkynyloxycarbonyl)amino, substituted or non-substituted (C₃-C₈-cycloalkoxycarbonyl)amino, substituted or non-substituted benzyloxy, substituted or non-substituted benzylsulphenyl, substituted or non-substituted benzylamino, substituted or non-substituted phenoxy, substituted or non-substituted phenylsulphenyl, substituted or non-substituted phenylamino, substituted or non-substituted aryl, substituted or non-substituted aryl-[C₁-C₈-alkyl.

According to the invention, processes P1 to P5 may be performed if appropriate in the presence of a solvent and if appropriate in the presence of a base.

Suitable solvents for carrying out processes P1 to P5 according to the invention are customary inert organic solvents. Preference is given to using optionally halogenated aliphatic, alicyclic or aromatic hydrocarbons, such as petroleum ether, hexane, heptane, cyclohexane, methylcyclohexane, benzene, toluene, xylene or decalin; chlorobenzene, dichlorobenzene, dichloromethane, chloroform, carbon tetrachloride, dichlorethane or trichlorethane; ethers, such as diethyl ether, diisopropyl ether, methyl tert-butyl ether, methyl tert-amyl ether, dioxane, tetrahydrofuran, 1,2-dimethoxyethane, 1,2-diethoxyethane or anisole; nitriles, such as acetonitrile, propionitrile, n- or iso-butyronitrile or benzonitrile; amides, such as N,N-dimethylformamide, N,N-dimethylacetamide, N-methylformanilide, N-methylpyrrolidone or hexamethylphosphoric triamide; esters, such as methyl acetate or ethyl acetate, sulphoxides, such as dimethyl sulphoxide, or sulphones, such as sulpholane.

Suitable bases for carrying out processes P1 to P5 according to the invention are inorganic and organic bases which are customary for such reactions. Preference is given to using alkaline earth metal, alkali metal hydride, alkali metal hydroxides or alkali metal alkoxides, such as sodium hydroxide, sodium hydride, calcium hydroxide, potassium hydroxide, potassium tert-butoxide or other ammonium hydroxide, alkali metal carbonates, such as sodium carbonate, potassium carbonate, potassium bicarbonate, sodium bicarbonate, cesium carbonate, alkali metal or alkaline earth metal acetates, such as sodium acetate, potassium acetate, calcium acetate, and also tertiary amines, such as trimethylamine, triethylamine, diisopropylethylamine, tributylamine, N,N-dimethylaniline, pyridine, N-methylpiperidine, N,N-dimethylaminopyridine, 1,4-diazabicyclo [2.2.2]octane (DABCO), 1,5-diazabicyclo[4.3.0]non-5-ene (DBN) or 1,8-diazabicyclo[5.4.0]undec-7-ene (DBU).

When carrying out processes P1 and P2 according to the invention, the reaction temperature can independently be varied within a relatively wide range. Generally, process P1 according to the invention is carried out at temperatures between 0° C. and 160° C.

Processes P1 and P2 according to the invention are generally independently carried out under atmospheric pressure. However, it is also possible to operate under elevated or reduced pressure.

When carrying out process P1 according to the invention, generally 1 mol or an excess of derivative of formula A-L¹-LG and from 1 to 3 mol of base are employed per mole of hydroximoyl tetrazole of formula (IVa), (IVb), (Va) or (Vb). It is also possible to employ the reaction components in other ratios.

Work-up is carried out by customary methods. Generally, the reaction mixture is treated with water and the organic phase is separated off and, after drying, concentrated under reduced pressure. If appropriate, the remaining residue can be freed by customary methods, such as chromatography or recrystallization, from any impurities that may still be present.

Compounds according to the invention can be prepared according to the above described processes. It will nevertheless be understood that, on the basis of his general knowledge and of available publications, the skilled worker will be able to adapt these processes according to the specifics of each of the compounds according to the invention that is desired to be synthesised.

Still in a further aspect, the present invention relates to compounds of formula (II) useful as intermediate compounds or materials for the process of preparation according to the invention. The present invention thus provides compounds of formula (II)

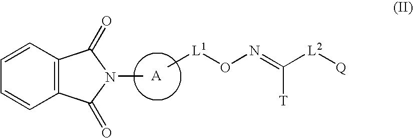

wherein
A, L¹, L², T, Q are as herein-defined

Still in a further aspect, the present invention relates to compounds of formula (IVa), (IVb), (Va), (Vb) useful as starting compounds or materials for the process of preparation according to the invention. The present invention thus provides compounds of formula (IVa):

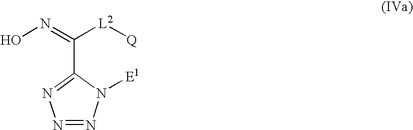

wherein L², Q, E¹ being as herein-defined.

The present invention thus provides compounds of formula (IVb):

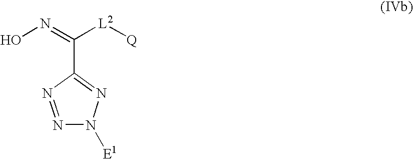

wherein L², Q, E¹ being as herein-defined.

The present invention thus provides compounds of formula (Va):

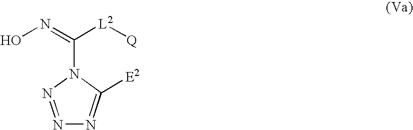

wherein L², Q, E² being as herein-defined.

The present invention thus provides compounds of formula (Vb):

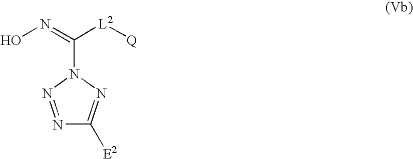

wherein L², Q, E² being as herein-defined.

The compounds of formula (IVa) and (IVb), useful as a starting material, can be prepared, for example, by reacting hydroxylamine with the corresponding ketones that can be prepared, for example, according to the method described by R. Raap (Can. J. Chem. 1971, 49, 2139) by addition of a tetrazolyl lithium species to esters of formula Q-L²-CO₂Me or Q-L²-CO₂Et, or any of their suitable synthetic equivalents like, for example: Q-L²-C(═O)—N(OMe)Me, Q-L²-CN, Q-L²-C(═O)Cl.

The compounds of general formula (Va) and (Vb), useful as a starting material, can be prepared, for example, from oximes of formula Q-L²-CH═N—OH and 5-substituted tetrazole according to the method described by J. Plenkiewicz et al. (Bull. Soc. Chien. Belg. 1987, 96, 675).

In a further aspect, the present invention also relates to a fungicide composition comprising an effective and non-phytotoxic amount of an active compound of formula (I) or (Ia) to (Id).

The expression “effective and non-phytotoxic amount” means an amount of composition according to the invention which is sufficient to control or destroy the fungi present or liable to appear on the crops, and which does not entail any appreciable symptom of phytotoxicity for the said crops. Such an amount can vary within a wide range depending on the fungus to be controlled, the type of crop, the climatic conditions and the compounds included in the fungicide composition according to the invention. This amount can be determined by systematic field trials, which are within the capabilities of a person skilled in the art.

Thus, according to the invention, there is provided a fungicide composition comprising, as an active ingredient, an effective amount of a compound of formula (I) or (Ia) to (Id) as herein defined and an agriculturally acceptable support, carrier or filler.

According to the invention, the term “support” denotes a natural or synthetic, organic or inorganic compound with which the active compound of formula (I) is combined or associated to make it easier to apply, notably to the parts of the plant. This support is thus generally inert and should be agriculturally acceptable. The support may be a solid or a liquid. Examples of suitable supports include clays, natural or synthetic silicates, silica, resins, waxes, solid fertilisers, water, alcohols, in particular butanol, organic solvents, mineral and plant oils and derivatives thereof. Mixtures of such supports may also be used.

The composition according to the invention may also comprise additional components. In particular, the composition may further comprise a surfactant. The surfactant can be an emulsifier, a dispersing agent or a wetting agent of ionic or non-ionic type or a mixture of such surfactants. Mention may be made, for example, of polyacrylic acid salts, lignosulphonic acid salts, phenolsulphonic or naphthalenesulphonic acid salts, polycondensates of ethylene oxide with fatty alcohols or with fatty acids or with fatty amines, substituted phenols (in particular alkylphenols or arylphenols), salts of sulphosuccinic acid esters, taurine derivatives (in particular alkyl taurates), phosphoric esters of polyoxyethylated alcohols or phenols, fatty acid esters of polyols, and derivatives of the above compounds containing sulphate, sulphonate and phosphate functions. The presence of at least one surfactant is generally essential when the active compound and/or the inert support are water-insoluble and when the vector agent for the application is water. Preferably, surfactant content may be comprised from 5% to 40% by weight of the composition.

Optionally, additional components may also be included, e.g. protective colloids, adhesives, thickeners, thixotropic agents, penetration agents, stabilisers, sequestering agents. More generally, the active compounds can be combined with any solid or liquid additive, which complies with the usual formulation techniques.

In general, the composition according to the invention may contain from 0.05 to 99% by weight of active compound, preferably 10 to 70% by weight.

Compositions according to the invention can be used in various forms such as aerosol dispenser, capsule suspension, cold fogging concentrate, dustable powder, emulsifiable concentrate, emulsion oil in water, emulsion water in oil, encapsulated granule, fine granule, flowable concentrate for seed treatment, gas (under pressure), gas generating product, granule, hot fogging concentrate, macrogranule, microgranule, oil dispersible powder, oil miscible flowable concentrate, oil miscible liquid, paste, plant rodlet, powder for dry seed treatment, seed coated with a pesticide, soluble concentrate, soluble powder, solution for seed treatment, suspension concentrate (flowable concentrate), ultra low volume (ULV) liquid, ultra low volume (ULV) suspension, water dispersible granules or tablets, water dispersible powder for slurry treatment, water soluble granules or tablets, water soluble powder for seed treatment and wettable powder. These compositions include not only compositions which are ready to be applied to the plant or seed to be treated by means of a suitable device, such as a spraying or dusting device, but also concentrated commercial compositions which must be diluted before application to the crop.

The compounds according to the invention can also be mixed with one or more insecticide, fungicide, bactericide, attractant, acaricide or pheromone active substance or other compounds with biological activity. The mixtures thus obtained have a broadened spectrum of activity. The mixtures with other fungicide compounds are particularly advantageous. The composition according to the invention comprising a mixture of a compound of formula (I) or (Ia) to (Id) with a bactericide compound may also be particularly advantageous

According to another object of the present invention, there is provided a method for controlling the phytopathogenic fungi of plants, crops or seeds, characterized in that an agronomically effective and substantially non-phytotoxic quantity of a pesticide composition according to the invention is applied as seed treatment, foliar application, stem application, drench or drip application (chemigation) to the seed, the plant or to the fruit of the plant or to soil or to inert substrate (e.g. inorganic substrates like sand, rockwool, glasswool; expanded minerals like perlite, vermiculite, zeolite or expanded clay), Pumice, Pyroclastic materials or stuff, synthetic organic substrates (e.g. polyurethane) organic substrates (e.g. peat, composts, tree waste products like coir, wood fibre or chips, tree bark) or to a liquid substrate (e.g. floating hydroponic systems, Nutrient Film Technique, Aeroponics) wherein the plant is growing or wherein it is desired to grow.

The expression “are applied to the plants to be treated” is understood to mean, for the purposes of the present invention, that the pesticide composition which is the subject of the invention can be applied by means of various methods of treatment such as:
spraying onto the aerial parts of the said plants a liquid comprising one of the said compositions,
dusting, the incorporation into the soil of granules or powders, spraying, around the said plants, and in the case of trees injection or daubing,
coating or film-coating the seeds of the said plants with the aid of a plant-protection mixture comprising one of the said compositions.

The method according to the invention may either be a curing, preventing or eradicating method.

In this method, a composition used can be prepared beforehand by mixing the two or more active compounds according to the invention.

According to an alternative of such a method, it is also possible to apply simultaneously, successively or separately compounds (A) and (B) so as to have the conjugated (A)/(B) effects, of distinct compositions each containing one of the two or three active ingredients (A) or (B).

The dose of active compound usually applied in the method of treatment according to the invention is generally and advantageously
for foliar treatments: from 0.1 to 10,000 g/ha, preferably from 10 to 1,000 g/ha, more preferably from 50 to 300 g/ha; in case of drench or drip application, the dose can even be reduced, especially while using inert substrates like rockwool or perlite;
for seed treatment: from 2 to 200 g per 100 kilogram of seed, preferably from 3 to 150 g per 100 kilogram of seed;
for soil treatment: from 0.1 to 10,000 g/ha, preferably from 1 to 5,000 g/ha.

The doses herein indicated are given as illustrative Examples of method according to the invention. A person skilled in the art will know how to adapt the application doses, notably according to the nature of the plant or crop to be treated.

Under specific conditions, for example according to the nature of the phytopathogenic fungus to be treated or controlled, a lower dose may offer adequate protection. Certain climatic conditions, resistance or other factors like the nature of the phytopathogenic fungi or the degree of infestation, for example, of the plants with these fungi, may require higher doses of combined active ingredients.

The optimum dose usually depends on several factors, for example on the type of phytopathogenic fungus to be treated, on the type or level of development of the infested plant, on the density of vegetation or alternatively on the method of application.

Without it being limiting, the crop treated with the pesticide composition or combination according to the invention is, for example, grapevine, but this could be cereals, vegetables, lucerne, soybean, market garden crops, turf, wood, tree or horticultural plants.

The method of treatment according to the invention may also be useful to treat propagation material such as tubers or rhizomes, but also seeds, seedlings or seedlings pricking out and plants or plants pricking out. This method of treatment may also be useful to treat roots. The method of treatment according to the invention may also be useful to treat the over-ground parts of the plant such as trunks, stems or stalks, leaves, flowers and fruit of the concerned plant.

Among the plants that can be protected by the method according to the invention, mention can be made of cotton; flax; vine; fruit or vegetable crops such as Rosaceae sp. (for instance pip fruit such as apples and pears, but also stone fruit such as apricots, almonds and peaches), Ribesioidae sp., Juglandaceae sp., Betulaceae sp., Anacardiaceae sp., Fagaceae sp., Moraceae sp., Oleaceae sp., Actinidaceae sp., Lauraceae sp., Musaceae sp. (for instance banana trees and plantins), Rubiaceae sp., Theaceae sp., Sterculiceae sp., Rutaceae sp. (for instance lemons oranges and grapefruit); Solanaceae sp. (for instance tomatoes), Liliaceae sp., Asteraceae sp. (for instance lettuces), Umbelliferae sp., Cruciferae sp., Chenopodiaceae sp., Cucurbitaceae sp., Papilionaceae sp. (for instance peas), Rosaceae sp. (for instance strawberries); major crops such as Graminae sp. (for instance maize, lawn or cereals such as wheat, rice, barley and triticale), Asteraceae sp. (for instance sunflower), Cruciferae sp. (for instance colza), Fabacae sp. (for instance peanuts), Papilionaceae sp. (for instance soybean), Solanaceae sp. (for instance potatoes), Chenopodiaceae sp. (for instance beetroots); horticultural and forest crops; as well as genetically modified homologues of these crops.

The product, composition and method of treatment according to the invention can be used in the treatment of genetically modified organisms (CMOs), e.g. plants or seeds. Genetically modified plants (or transgenic plants) are plants of which a heterologous gene has been stably integrated into genome. The expression “heterologous gene” essentially means a gene which is provided or assembled outside the plant and when introduced in the nuclear, chloroplastic or mitochondrial genome gives the transformed plant new or improved agronomic or other properties by expressing a protein or polypeptide of interest or by downregulating or silencing other gene(s) which are present in the plant (using for example, antisense technology, cosuppression technology or RNA interference—RNAi—technology). A heterologous gene that is located in the genome is also called a transgene. A transgene that is defined by its particular location in the plant genome is called a transformation or transgenic event.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the active compounds and compositions which can be used according to the invention, better plant growth, increased tolerance to high or low temperatures, increased tolerance to drought or to water or soil salt content, increased flowering performance, easier harvesting, accelerated maturation, higher harvest yields, bigger fruits, larger plant height, greener leaf color, earlier flowering, higher quality and/or a higher nutritional value of the harvested products, higher sugar concentration within the fruits, better storage stability and/or processability of the harvested products are possible, which exceed the effects which were actually to be expected.

At certain application rates, the active compound combinations according to the invention may also have a strengthening effect in plants. Accordingly, they are also suitable for mobilizing the defense system of the plant against attack by unwanted microorganisms. This may, if appropriate, be one of the reasons of the enhanced activity of the combinations according to the invention, for example against fungi. Plant-strengthening (resistance-inducing) substances are to be understood as meaning, in the present context, those substances or combinations of substances which are capable of stimulating the defense system of plants in such a way that, when subsequently inoculated with unwanted microorganisms, the treated plants display a substantial degree of resistance to these microorganisms. In the present case, unwanted microorganisms are to be understood as meaning phytopathogenic fungi, bacteria and viruses. Thus, the substances according to the invention can be employed for protecting plants against attack by the abovementioned pathogens within a certain period of time after the treatment. The period of time within which protection is effected generally extends from 1 to 10 days, preferably 1 to 7 days, after the treatment of the plants with the active compounds.

Plants and plant cultivars which are preferably to be treated according to the invention include all plants which have genetic material which impart particularly advantageous, useful traits to these plants (whether obtained by breeding and/or biotechnological means).

Plants and plant cultivars which are also preferably to be treated according to the invention are resistant against one or more biotic stresses, i.e. said plants show a better defense against animal and microbial pests, such as against nematodes, insects, mites, phytopathogenic fungi, bacteria, viruses and/or viroids.

Plants and plant cultivars which may also be treated according to the invention are those plants which are resistant to one or more abiotic stresses. Abiotic stress conditions may include, for example, drought, cold temperature exposure, heat exposure, osmotic stress, flooding, increased soil salinity, increased mineral exposure, ozone exposure, high light exposure, limited availability of nitrogen nutrients, limited availability of phosphorus nutrients, shade avoidance.

Plants and plant cultivars which may also be treated according to the invention, are those plants characterized by enhanced yield characteristics. Increased yield in said plants can be the result of, for example, improved plant physiology, growth and development, such as water use efficiency, water retention efficiency, improved nitrogen use, enhanced carbon assimilation, improved photosynthesis, increased germination efficiency and accelerated maturation. Yield can furthermore be affected by improved plant architecture (under stress and non-stress conditions), including but not limited to, early flowering, flowering control for hybrid seed production, seedling vigor, plant size, internode number and distance, root growth, seed size, fruit size, pod size, pod or ear number, seed number per pod or ear, seed mass, enhanced seed filling, reduced seed dispersal, reduced pod dehiscence and lodging resistance. Further yield traits include seed composition, such as carbohydrate content, protein content, oil content and composition, nutritional value, reduction in anti-nutritional compounds, improved processability and better storage stability.

Non-exhaustive examples of plants with the above-mentioned traits are disclosed in the references listed in Table A.

TABLE A

| Trait | Reference |
|---|---|
| Water use efficiency | WO 2000/073475 |
| Nitrogen use efficiency | WO 1995/009911; WO 1997/030163; WO 2007/092704; WO |
| | 2007/076115; WO 2005/103270; WO 2002/002776 |
| Improved photosynthesis | WO 2008/056915; WO 2004/101751 |
| Nematode resistance | WO 1995/020669; WO 2001/051627; WO 2008/139334; WO |
| | 2008/095972; WO 2006/085966; WO 2003/033651; WO |
| | 1999/060141; WO 1998/012335; WO 1996/030517; WO |
| | 1993/018170 |
| Reduced pod dehiscence | WO 2006/009649; WO 2004/113542; WO 1999/015680; WO |
| | 1999/000502; WO 1997/013865; WO 1996/030529; WO |
| | 1994/023043 |
| Aphid resistance | WO 2006/125065; WO 1997/046080; WO 2008/067043; WO |
| | 2004/072109 |
| Sclerotinia resistance | WO 2006/135717; WO 2006/055851; WO 2005/090578; WO |
| | 2005/000007; WO 2002/099385; WO 2002/061043 |
| Botrytis resistance | WO 2006/046861; WO 2002/085105 |
| Bremia resistance | US 20070022496; WO 2000/063432; WO 2004/049786 |
| Erwinia resistance | WO 2004/049786 |
| Closterovirus resistance | WO 2007/073167; WO 2007/053015; WO 2002/022836 |
| Tobamovirus resistance | WO 2006/038794 |

Plants that may be treated according to the invention are hybrid plants that already express the characteristic of heterosis or hybrid vigor which results in generally higher yield, vigor, health and resistance towards biotic and abiotic stresses). Such plants are typically made by crossing an inbred male-sterile parent line (the female parent) with another inbred male-fertile parent line (the male parent). Hybrid seed is typically harvested from the male sterile plants and sold to growers. Male sterile plants can sometimes (e.g. in corn) be produced by detasseling, i.e. the mechanical removal of the male reproductive organs (or males flowers) but, more typically, male sterility is the result of genetic determinants in the plant genome. In that case, and especially when seed is the desired product to be harvested from the hybrid plants it is typically useful to ensure that male fertility in the hybrid plants is fully restored. This can be accomplished by ensuring that the male parents have appropriate fertility restorer genes which are capable of restoring the male fertility in hybrid plants that contain the genetic determinants responsible for male-sterility. Genetic determinants for male sterility may be located in the cytoplasm. Examples of cytoplasmic male sterility (CMS) were for instance described in Brassica species (WO 92/05251, WO 95/09910, WO 98/27806, WO 05/002324, WO 06/021972 and U.S. Pat. No. 6,229,072). However, genetic determinants for male sterility can also be located in the nuclear genome. Male sterile plants can also be obtained by plant biotechnology methods such as genetic engineering. A particularly useful means of obtaining male-sterile plants is described in WO 89/10396 in which, for example, a ribonuclease such as barnase is selectively expressed in the tapetum cells in the stamens. Fertility can then be restored by expression in the tapetum cells of a ribonuclease inhibitor such as barstar (e.g. WO 91/02069).

Plants or plant cultivars (obtained by plant biotechnology methods such as genetic engineering) which may be treated according to the invention are herbicide-tolerant plants, i.e. plants made tolerant to one or more given herbicides. Such plants can be obtained either by genetic transformation, or by selection of plants containing a mutation imparting such herbicide tolerance.

Herbicide-resistant plants are for example glyphosate-tolerant plants, i.e. plants made tolerant to the herbicide glyphosate or salts thereof. Plants can be made tolerant to glyphosate through different means. For example, glyphosate-tolerant plants can be obtained by transforming the plant with a gene encoding the enzyme 5-enolpyruvylshikimate-3-phosphate synthase (EPSPS). Examples of such EPSPS genes are the AroA gene (mutant CT7) of the bacterium Salmonella typhimurium (Comai et al., 1983, Science 221, 370-371), the CP4 gene of the bacterium Agrobacterium sp. (Barry et al., 1992, Curr. Topics Plant Physiol. 7, 139-145), the genes encoding a Petunia EPSPS (Shah et al., 1986, Science 233, 478-481), a Tomato EPSPS (Gasser et al., 1988, J. Biol. Chem. 263, 4280-4289), or an Eleusine EPSPS (WO 01/66704). It can also be a mutated EPSPS as described in for example EP 0837944, WO 00/66746, WO 00/66747 or WO02/26995. Glyphosate-tolerant plants can also be obtained by expressing a gene that encodes a glyphosate oxido-reductase enzyme as described in U.S. Pat. Nos. 5,776,760 and 5,463,175. Glyphosate-tolerant plants can also be obtained by expressing a gene that encodes a glyphosate acetyl transferase enzyme as described in for example WO 02/36782, WO 03/092360, WO 05/012515 and WO 07/024782. Glyphosate-tolerant plants can also be obtained by selecting plants containing naturally-occurring mutations of the above-mentioned genes, as described in for example WO 01/024615 or WO 03/013226.

Other herbicide resistant plants are for example plants that are made tolerant to herbicides inhibiting the enzyme glutamine synthase, such as bialaphos, phosphinothricin or glufosinate. Such plants can be obtained by expressing an enzyme detoxifying the herbicide or a mutant glutamine synthase enzyme that is resistant to inhibition. One such efficient detoxifying enzyme is an enzyme encoding a phosphinothricin acetyltransferase (such as the bar or pat protein from Streptomyces species). Plants expressing an exogenous phosphinothricin acetyltransferase are for example described in U.S. Pat. Nos. 5,561,236; 5,648,477; 5,646,024; 5,273,894; 5,637,489; 5,276,268; 5,739,082; 5,908,810 and 7,112,665.

Further herbicide-tolerant plants are also plants that are made tolerant to the herbicides inhibiting the enzyme hydroxyphenylpyruvatedioxygenase (HPPD). Hydroxyphenylpyruvatedioxygenases are enzymes that catalyze the reaction in which para-hydroxyphenylpyruvate (HPP) is transformed into homogentisate. Plants tolerant to HPPD-inhibitors can be transformed with a gene encoding a naturally-occurring resistant HPPD enzyme, or a gene encoding a mutated HPPD enzyme as described in WO 96/38567, WO 99/24585 and WO 99/24586. Tolerance to HPPD-inhibitors can also be obtained by transforming plants with genes encoding certain enzymes enabling the formation of homogentisate despite the inhibition of the native HPPD enzyme by the HPPD-inhibitor. Such plants and genes are described in WO 99/34008 and WO 02/36787. Tolerance of plants to HPPD inhibitors can also be improved by transforming plants with a gene encoding an enzyme prephenate deshydrogenase in addition to a gene encoding an HPPD-tolerant enzyme, as described in WO 2004/024928.

Still further herbicide resistant plants are plants that are made tolerant to acetolactate synthase (ALS) inhibitors. Known ALS-inhibitors include, for example, sulfonylurea, imidazolinone, triazolopyrimidines, pryimidinyoxy(thio)benzoates, and/or sulfonylaminocarbonyltriazolinone herbicides. Different mutations in the ALS enzyme (also known as acetohydroxyacid synthase, AHAS) are known to confer tolerance to different herbicides and groups of herbicides, as described for example in Tranel and Wright (2002, Weed Science 50:700-712), but also, in U.S. Pat. Nos. 5,605,011, 5,378,824, 5,141,870, and 5,013,659. The production of sulfonylurea-tolerant plants and imidazolinone-tolerant plants is described in U.S. Pat. Nos. 5,605,011; 5,013,659; 5,141,870; 5,767,361; 5,731,180; 5,304,732; 4,761,373; 5,331,107; 5,928,937; and 5,378,824; and international publication WO 96/33270. Other imidazolinone-tolerant plants are also described in for example WO 2004/040012, WO 2004/106529, WO 2005/020673, WO 2005/093093, WO 2006/007373, WO 2006/015376, WO 2006/024351, and WO 2006/060634. Further sulfonylurea- and imidazolinone-tolerant plants are also described in for example WO 07/024782.

Other plants tolerant to imidazolinone and/or sulfonylurea can be obtained by induced mutagenesis, selection in cell cultures in the presence of the herbicide or mutation breeding as described for example for soybeans in U.S. Pat. No. 5,084,082, for rice in WO 97/41218, for sugar beet in U.S. Pat. No. 5,773,702 and WO 99/057965, for lettuce in U.S. Pat. No. 5,198,599, or for sunflower in WO 01/065922.

Plants or plant cultivars (obtained by plant biotechnology methods such as genetic engineering) which may also be treated according to the invention are insect-resistant transgenic plants, i.e. plants made resistant to attack by certain target insects. Such plants can be obtained by genetic transformation, or by selection of plants containing a mutation imparting such insect resistance.

An “insect-resistant transgenic plant”, as used herein, includes any plant containing at least one transgene comprising a coding sequence encoding:
1) an insecticidal crystal protein from Bacillus thuringiensis or an insecticidal portion thereof, such as the insecticidal crystal proteins listed by Crickmore et al. (1998, Microbiology and Molecular Biology Reviews, 62: 807-813), updated by Crickmore et al. (2005) at the Bacillus thuringiensis toxin nomenclature, online at: http://www.lifesci.sussex.ac.uk/Home/Neil_Crickmore/Bt/), or insecticidal portions thereof, e.g., proteins of the Cry protein classes Cry1Ab, Cry1Ac, Cry1B, Cry1C, Cry1D, Cry1F, Cry2Ab, Cry3Aa, or Cry3Bb or insecticidal portions thereof (e.g. EP 1999141 and WO 2007/107302); or
2) a crystal protein from Bacillus thuringiensis or a portion thereof which is insecticidal in the presence of a second other crystal protein from Bacillus thuringiensis or a portion thereof, such as the binary toxin made up of the Cry34 and Cry35 crystal proteins (Moellenbeck et al. 2001, Nat. Biotechnol. 19: 668-72; Schnepf et al. 2006, Applied Environm. Microbiol. 71, 1765-1774) or the binary toxin made up of the Cry1A or Cry1F proteins and the Cry2Aa or Cry2Ab or Cry2Ae proteins (U.S. patent application Ser. No. 12/214,022 and EP 08010791.5); or
3) a hybrid insecticidal protein comprising parts of different insecticidal crystal proteins from Bacillus thuringiensis, such as a hybrid of the proteins of 1) above or a hybrid of the proteins of 2) above, e.g., the Cry1A.105 protein produced by corn event MON89034 (WO 2007/027777); or
4) a protein of any one of 1) to 3) above wherein some, particularly 1 to 10, amino acids have been replaced by another amino acid to obtain a higher insecticidal activity to a target insect species, and/or to expand the range of target insect species affected, and/or because of changes introduced into the encoding DNA during cloning or transformation, such as the Cry3Bb1 protein in corn events MON863 or MON88017, or the Cry3A protein in corn event MIR604; or
5) an insecticidal secreted protein from Bacillus thuringiensis or Bacillus cereus, or an insecticidal portion thereof, such as the vegetative insecticidal (VIP) proteins listed at: http://www.lifesci.sussex.ac.uk/home/Neil_Crickmore/Bt/vip.html, e.g., proteins from the VIP3Aa protein class; or
6) a secreted protein from Bacillus thuringiensis or Bacillus cereus which is insecticidal in the presence of a second secreted protein from Bacillus thuringiensis or B. cereus, such as the binary toxin made up of the VIP1A and VIP2A proteins (WO 94121795); or
7) a hybrid insecticidal protein comprising parts from different secreted proteins from Bacillus thuringiensis or Bacillus cereus, such as a hybrid of the proteins in 1) above or a hybrid of the proteins in 2) above; or
8) a protein of any one of 5) to 7) above wherein some, particularly 1 to 10, amino acids have been replaced by another amino acid to obtain a higher insecticidal activity to a target insect species, and/or to expand the range of target insect species affected, and/or because of changes introduced into the encoding DNA during cloning or transformation (while still encoding an insecticidal protein), such as the VIP3Aa protein in cotton event COT102; or
9) a secreted protein from Bacillus thuringiensis or Bacillus cereus which is insecticidal in the presence of a crystal protein from Bacillus thuringiensis, such as the binary toxin made up of VIP3 and Cry1A or Cry1F (U.S. Patent Appl. No. 61/126,083 and 61/195,019), or the binary toxin made up of the VIP3 protein and the Cry2Aa or Cry2Ab or Cry2Ae proteins (U.S. patent application Ser. No. 12/214,022 and EP 08010791.5).
10) a protein of 9) above wherein some, particularly 1 to 10, amino acids have been replaced by another amino acid to obtain a higher insecticidal activity to a target insect species, and/or to expand the range of target insect species affected, and/or because of changes introduced into the encoding DNA during cloning or transformation (while still encoding an insecticidal protein)

Of course, an insect-resistant transgenic plant, as used herein, also includes any plant comprising a combination of genes encoding the proteins of any one of the above classes 1 to 10. In one embodiment, an insect-resistant plant contains more than one transgene encoding a protein of any one of the above classes 1 to 10, to expand the range of target insect species affected when using different proteins directed at different target insect species, or to delay insect resistance development to the plants by using different proteins insecticidal to the same target insect species but having a different mode of action, such as binding to different receptor binding sites in the insect.

An “insect-resistant transgenic plant”, as used herein, further includes any plant containing at least one transgene comprising a sequence producing upon expression a double-stranded RNA which upon ingestion by a plant insect pest inhibits the growth of this insect pest, as described e.g. in WO 2007/080126.

Plants or plant cultivars (obtained by plant biotechnology methods such as genetic engineering) which may also be treated according to the invention are tolerant to abiotic stresses. Such plants can be obtained by genetic transformation, or by selection of plants containing a mutation imparting such stress resistance. Particularly useful stress tolerance plants include:
1) plants which contain a transgene capable of reducing the expression and/or the activity of poly(ADP-ribose) polymerase (PARP) gene in the plant cells or plants as described in WO 00/04173, WO/2006/045633, EP 04077984.5, or EP 06009836.5.
2) plants which contain a stress tolerance enhancing transgene capable of reducing the expression and/or the activity of the PARG encoding genes of the plants or plants cells, as described e.g. in WO 2004/090140.
3) plants which contain a stress tolerance enhancing transgene coding for a plant-functional enzyme of the nicotineamide adenine dinucleotide salvage synthesis pathway including nicotinamidase, nicotinate phosphoribosyltransferase, nicotinic acid mononucleotide adenyl transferase, nicotinamide adenine dinucleotide synthetase or nicotine amide phosphorybosyltransferase as described e.g. in EP 04077624.7, WO 2006/133827, PCT/EP07/002433, EP 1999263, or WO 2007/107326.

Plants or plant cultivars (obtained by plant biotechnology methods such as genetic engineering) which may also be treated according to the invention show altered quantity, quality and/or storage-stability of the harvested product and/or altered properties of specific ingredients of the harvested product such as
1) transgenic plants which synthesize a modified starch, which in its physical-chemical characteristics, in particular the amylose content or the amylose/amylopectin ratio, the degree of branching, the average chain length, the side chain distribution, the viscosity behaviour, the gelling strength, the starch grain size and/or the starch grain morphology, is changed in comparison with the synthesised starch in wild type plant cells or plants, so that this is better suited for special applications. Said transgenic plants synthesizing a modified starch are disclosed, for example, in EP 0571427, WO 95/04826, EP 0719338, WO 96/15248, WO 96/19581, WO 96/27674, WO 97/11188, WO 97/26362, WO 97/32985, WO 97/42328, WO 97/44472, WO 97/45545, WO 98/27212, WO 98/40503, W099/58688, WO 99/58690, WO 99/58654, WO 00/08184, WO 00/08185, WO 00/08175, WO 00/28052, WO 00/77229, WO 01/12782, WO 01/12826, WO 02/101059, WO 03/071860, WO 2004/056999, WO 2005/030942, WO 2005/030941, WO 2005/095632, WO 2005/095617, WO 2005/095619, WO 2005/095618, WO 2005/123927, WO 2006/018319, WO 2006/103107, WO 2006/108702, WO 2007/009823, WO 00/22140, WO 2006/063862, WO 2006/072603, WO 02/034923, EP 06090134.5, EP 06090228.5, EP 06090227.7, EP 07090007.1, EP 07090009.7, WO 01/14569, WO 02/79410, WO 03/33540, WO 2004/078983, WO 01/19975, WO 95/26407, WO 96/34968, WO 98/20145, WO 99/12950, WO 99/66050, WO 99/53072, U.S. Pat. No. 6,734,341, WO 00/11192, WO 98/22604, WO 98/32326, WO 01/98509, WO 01/98509, WO 2005/002359, U.S. Pat. No. 5,824,790, U.S. Pat. No. 6,013,861, WO 94/04693, WO 94/09144, WO 94/11520, WO 95/35026, WO 97/20936
2) transgenic plants which synthesize non starch carbohydrate polymers or which synthesize non starch carbohydrate polymers with altered properties in comparison to wild type plants without genetic modification. Examples are plants producing polyfructose, especially of the inulin and levan-type, as disclosed in EP 0663956, WO 96/01904, WO 96/21023, WO 98/39460, and WO 99/24593, plants producing alpha-1,4-glucans as disclosed in WO 95/31553, US 2002031826, U.S. Pat. No. 6,284,479, U.S. Pat. No. 5,712,107, WO 97/47806, WO 97/47807, WO 97/47808 and WO 00/14249, plants producing alpha-1,6 branched alpha-1,4-glucans, as disclosed in WO 00/73422, plants producing alternan, as disclosed in e.g. WO 00/47727, WO 00/73422, EP 06077301.7, U.S. Pat. No. 5,908,975 and EP 0728213,
3) transgenic plants which produce hyaluronan, as for example disclosed in WO 2006/032538, WO 2007/039314, WO 2007/039315, WO 2007/039316, JP 2006304779, and WO 2005/012529.
4) transgenic plants or hybrid plants, such as onions with characteristics such as ‘high soluble solids content’, ‘low pungency’ (LP) and/or ‘long storage’ (LS), as described in U.S. patent application Ser. Nos. 12/020,360 and 61/054,026.

Plants or plant cultivars (that can be obtained by plant biotechnology methods such as genetic engineering) which may also be treated according to the invention are plants, such as cotton plants, with altered fiber characteristics. Such plants can be obtained by genetic transformation, or by selection of plants contain a mutation imparting such altered fiber characteristics and include:
a) Plants, such as cotton plants, containing an altered form of cellulose synthase genes as described in WO 98/00549
b) Plants, such as cotton plants, containing an altered form of rsw2 or rsw3 homologous nucleic acids as described in WO 2004/053219
c) Plants, such as cotton plants, with increased expression of sucrose phosphate synthase as described in WO 01/17333
d) Plants, such as cotton plants, with increased expression of sucrose synthase as described in WO 02/45485
e) Plants, such as cotton plants, wherein the timing of the plasmodesmatal gating at the basis of the fiber cell is altered, e.g. through downregulation of fiber-selective β-1,3-glucanase as described in WO 2005/017157, or as described in EP 08075514.3 or U.S. Patent Appl. No. 61/128,938
f) Plants, such as cotton plants, having fibers with altered reactivity, e.g. through the expression of N-acetylglucosaminetransferase gene including nodC and chitin synthase genes as described in WO 2006/136351

Plants or plant cultivars (that can be obtained by plant biotechnology methods such as genetic engineering) which may also be treated according to the invention are plants, such as oilseed rape or related Brassica plants, with altered oil profile characteristics. Such plants can be obtained by genetic transformation, or by selection of plants contain a mutation imparting such altered oil profile characteristics and include:
a) Plants, such as oilseed rape plants, producing oil having a high oleic acid content as described e.g. in U.S. Pat. No. 5,969,169, U.S. Pat. No. 5,840,946 or U.S. Pat. No. 6,323,392 or U.S. Pat. No. 6,063,947
b) Plants such as oilseed rape plants, producing oil having a low linolenic acid content as described in U.S. Pat. No. 6,270,828, U.S. Pat. No. 6,169,190 or U.S. Pat. No. 5,965,755
c) Plant such as oilseed rape plants, producing oil having a low level of saturated fatty acids as described e.g. in U.S. Pat. No. 5,434,283

Plants or plant cultivars (that can be obtained by plant biotechnology methods such as genetic engineering) which may also be treated according to the invention are plants, such as oilseed rape or related Brassica plants, with altered seed shattering characteristics. Such plants can be obtained by genetic transformation, or by selection of plants contain a mutation imparting such altered seed shattering characteristics and include plants such as oilseed rape plants with delayed or reduced seed shattering as described in U.S. Patent Appl. No. 61/135,230 and EP 08075648.9.

Particularly useful transgenic plants which may be treated according to the invention are plants containing transformation events, or combination of transformation events, that are the subject of petitions for non-regulated status, in the United States of America, to the Animal and Plant Health Inspection Service (APHIS) of the United States Department of Agriculture (USDA) whether such petitions are granted or are still pending. At any time this information is readily available from APHIS (4700 River Road Riverdale, Md. 20737, USA), for instance on its internet site (URL http://www.aphis.usda.gov/brs/not_reg.html).

Additional particularly useful plants containing single transformation events or combinations of transformation events are listed for example in the databases from various national or regional regulatory agencies (see for example http://gmoinfo.jrc.it/gmp_browse.aspx and http://www.agbios.com/dbase.php).

Further particularly transgenic plants include plants containing a transgene in an agronomically neutral or beneficial position as described in any of the patent publications listed in Table C.

TABLE C

| Plant species | Trait | Patent reference |
|---|---|---|
| Corn | Glyphosate tolerance | US 2007-056056 |
| Corn | Insect resistance (Cry3a055) | EP 1 737 290 |
| Corn | High lysine content | U.S. Pat. No. 7,157,281 |
| Corn | Self processing corn | US 2006-230473 |
| | (alpha-amylase) | |
| Corn | Insect resistance (Cry3Bb) | US 2006-095986 |
| Corn | Insect resistance | US 2006-070139 |
| | (Cry34Ab1/Cry35Ab1) | |
| Corn | Insect resistance (Cry1F) | U.S. Pat. No. 7,435,807 |
| Corn | Insect resistance (Cry1Ab) | US 2004-180373 |
| Corn | Insect resistance | WO 03/052073 |
| Corn | Glufosinate resistance | US 2003-126634 |
| Corn | Glyphosate resistance | U.S. Pat. No. 6,040,497 |
| Corn | Glyphosate resistance | U.S. Pat. No. 6,040,497 |
| Corn | Glyphosate resistance | U.S. Pat. No. 6,040,497 |
| Corn | Glyphosate resistance | U.S. Pat. No. 6,040,497 |
| Corn | Glufosinate tolerance | WO 01/51654 |
| Corn | Glyphosate tolerance/ALS | WO 2008/112019 |
| | inhibitor tolerance | |
| Wheat | Fusarium resistance | CA 2561992 |
| | (trichothecene 3-O- | |
| | acetyltransferase) | |
| Sugar beet | Glyphosate tolerance | US 2004-117870 |
| Sugar beet | Glyphosate tolerance | WO 2004- |
| | | 074492 |
| Soybean | Glyphosate tolerance | US 2006-282915 |
| Soybean | Glufosinate tolerance | WO |
| | | 2006/108674 |
| Soybean | Glufosinate tolerance | WO |
| | | 2006/108675 |
| Soybean | High oleic acid/ALS | WO |
| | inhibitor tolerance | 2008/054747 |
| Rice | Glufosinate tolerance | WO 01/83818 |
| Rice | Glufosinate tolerance | US 2008-289060 |
| Rice | Insect resistance (Cry1Ac) | WO |
| | | 2008/114282 |
| Oilseed rape | Male sterility | WO 01/31042 |
| Oilseed rape | Male sterility/restoration | WO 01/41558 |
| Oilseed rape | Glyphosate resistance | WO 02/36831 |
| Cotton | Insect resistance (Cry1Ab) | WO |
| | | 2006/128573 |
| Cotton | Insect resistance (Cry1Ab) | WO |
| | | 2006/128572 |
| Cotton | Insect resistance (Cry1Ab) | WO |
| | | 2006/128571 |
| Cotton | Insect resistance (Cry1Ab) | WO |
| | | 2006/128569 |
| Cotton | Insect resistance (Cry1Ab) | WO |
| | | 2006/128570 |
| Cotton | Insect resistance (Cry1Ab) | WO |
| | | 2006/128568 |
| Cotton | Insect resistance (Cry1Ac) | WO |
| | | 2005/103266 |
| Cotton | Glyphosate tolerance | US 2004-148666 |
| Cotton | Glyphosate tolerance | WO |
| | | 2004/072235 |
| Cotton | Glyphosate tolerance | WO |
| | | 2007/017186 |
| Cotton | Insect-resistance (Cry1Ab) | WO2008/122406 |
| Cotton | Insect resistance (VIP3) | US 2007-067868 |
| Cotton | Glufosinate resistance | WO |
| | | 2007/017186 |
| Cotton | Insect resistance (Cry1Ab) | WO |
| | | 2008/122406 |
| Cotton | Insect resistance (Cry1F) | WO |
| | | 2005/103266 |
| Cotton | Insect resistance (Vip3A) | US 2006-130175 |
| Cotton | Insect resistance | US 2004-250317 |
| | (Cry1A/Cry2Ab) | |
| Bent Grass | Glyphosate tolerance | US 2006-162007 |
| Brinjal | Insect resistance (Cry1Ac) | WO |
| | | 2007/091277 |

The composition according to the invention may also be used against fungal diseases liable to grow on or inside timber. The term “timber” means all types of species of wood, and all types of working of this wood intended for construction, for example solid wood, high-density wood, laminated wood, and plywood. The method for treating timber according to the invention mainly consists in contacting one or more compounds according to the invention or a composition according to the invention; this includes for example direct application, spraying, dipping, injection or any other suitable means.

Among the diseases of plants or crops that can be controlled by the method according to the invention, mention can be made of:

Powdery mildew diseases such as:
Blumeria diseases, caused for example by Blumeria graminis;
Podosphaera diseases, caused for example by Podosphaera leucotricha;
Sphaerotheca diseases, caused for example by Sphaerotheca fuliginea;
Uncinula diseases, caused for example by Uncinula necator;

Rust diseases such as:
Gymnosporangium diseases, caused for example by Gymnosporangium sabinae;
Hemileia diseases, caused for example by Hemileia vastatrix;
Phakopsora diseases, caused for example by Phakopsora pachyrhizi or Phakopsora meibomiae;
Puccinia diseases, caused for example by Puccinia recondite, Puccinia graminis or Puccinia striiformis;
Uromyces diseases, caused for example by Uromyces appendiculatus;

Oomycete diseases such as:
Albugo diseases caused for example by Albugo candida;
Bremia diseases, caused for example by Bremia lactucae;
Peronospora diseases, caused for example by Peronospora pisi or P. brassicae;
Phytophthora diseases, caused for example by Phytophthora infestans;
Plasmopara diseases, caused for example by Plasmopara viticola;
Pseudoperonospora diseases, caused for example by Pseudoperonospora humuli or Pseudoperonospora cubensis;
Pythium diseases, caused for example by Pythium ultimum;

Leafspot, leaf blotch and leaf blight diseases such as:
Alternaria diseases, caused for example by Alternaria solani;
Cercospora diseases, caused for example by Cercospora beticola;
Cladiosporum diseases, caused for example by Cladiosporium cucumerinum;
Cochliobolus diseases, caused for example by Cochliobolus sativus (Conidiaform: Drechslera, Syn: Helminthosporium) or Cochliobolus miyabeanus;
Colletotrichum diseases, caused for example by Colletotrichum lindemuthanium;
Cycloconium diseases, caused for example by Cycloconium oleaginum;
Diaporthe diseases, caused for example by Diaporthe citri;
Elsinoe diseases, caused for example by Elsinoe fawcettii;
Gloeosporium diseases, caused for example by Gloeosporium laeticolor;
Glomerella diseases, caused for example by Glomerella cingulata;
Guignardia diseases, caused for example by Guignardia bidwelli;
Leptosphaeria diseases, caused for example by Leptosphaeria maculans; Leptosphaeria nodorum;
Magnaporthe diseases, caused for example by Magnaporthe grisea;
Mycosphaerella diseases, caused for example by Mycosphaerella graminicola; Mycosphaerella arachidicola; Mycosphaerella fijiensis;
Phaeosphaeria diseases, caused for example by Phaeosphaeria nodorum;
Pyrenophora diseases, caused for example by Pyrenophora teres, or Pyrenophora tritici repentis;
Ramularia diseases, caused for example by Ramularia collo-cygni, or Ramularia areola;
Rhynchosporium diseases, caused for example by Rhynchosporium secalis;
Septoria diseases, caused for example by Septoria apii or Septoria lycopercisi;
Typhula diseases, caused for example by Typhula incarnata;
Venturia diseases, caused for example by Venturia inaequatis;

Root, Sheath and stem diseases such as:
Corticium diseases, caused for example by Corticium graminearum;
Fusarium diseases, caused for example by Fusarium oxysporum;
Gaeumannomyces diseases, caused for example by Gaeumannomyces graminis;
Rhizoctonia diseases, caused for example by Rhizoctonia solani;
Sarocladium diseases caused for example by Sarocladium oryzae;
Sclerotium diseases caused for example by Sclerotium oryzae;
Tapesia diseases, caused for example by Tapesia acuformis;
Thielaviopsis diseases, caused for example by Thielaviopsis basicola;

Ear and panicle diseases such as:
Alternaria diseases, caused for example by Alternaria spp.;
Aspergillus diseases, caused for example by Aspergillus flavus;
Cladosporium diseases, caused for example by Cladosporium spp.;
Claviceps diseases, caused for example by Claviceps purpurea;
Fusarium diseases, caused for example by Fusarium culmorum;
Gibberella diseases, caused for example by Gibberella zeae;
Monographella diseases, caused for example by Monographella nivalis;

Smut and bunt diseases such as:
Sphacelotheca diseases, caused for example by Sphacelotheca reiliana;
Tilletia diseases, caused for example by Tilletia caries;
Urocystis diseases, caused for example by Urocystis occulta;
Ustilago diseases, caused for example by Ustilago nuda;

Fruit rot and mould diseases such as:
Aspergillus diseases, caused for example by Aspergillus flavus;
Botrytis diseases, caused for example by Botrytis cinerea;
Penicillium diseases, caused for example by Penicillium expansum;
Rhizopus diseases caused by example by Rhizopus stolonifer
Sclerotinia diseases, caused for example by Sclerotinia sclerotiorum;
Verticilium diseases, caused for example by Verticilium alboatrum;

Seed and soilborne decay, mould, wilt, rot and damping-off diseases:
Alternaria diseases, caused for example by Alternaria brassicicola
Aphanomyces diseases, caused for example by Aphanomyces euteiches
Ascochyta diseases, caused for example by Ascochyta lentis
Aspergillus diseases, caused for example by Aspergillus flavus
Cladosporium diseases, caused for example by Cladosporium herbarum
Cochliobolus diseases, caused for example by Cochliobolus sativus (Conidiaform: Drechslera, Bipolaris Syn: Helminthosporium);
Colletotrichum diseases, caused for example by Colletotrichum coccodes;
Fusarium diseases, caused for example by Fusarium culmorum;
Gibberella diseases, caused for example by Gibberella zeae;
Macrophomina diseases, caused for example by Macrophomina phaseolina
Monographella diseases, caused for example by Monographella nivalis;
Penicillium diseases, caused for example by Penicillium expansum
Phoma diseases, caused for example by Phoma lingam
Phomopsis diseases, caused for example by Phomopsis sojae;
Phytophthora diseases, caused for example by Phytophthora cactorum;
Pyrenophora diseases, caused for example by Pyrenophora graminea
Pyricularia diseases, caused for example by Pyricularia oryzae;
Pythium diseases, caused for example by Pythium ultimum;
Rhizoctonia diseases, caused for example by Rhizoctonia solani;
Rhizopus diseases, caused for example by Rhizopus oryzae
Sclerotium diseases, caused for example by Sclerotium rolfsii;
Septoria diseases, caused for example by Septoria nodorum;
Typhula diseases, caused for example by Typhula incarnata;
Verticillium diseases, caused for example by Verticillium dahliae;

Canker, broom and dieback diseases such as:
Nectria diseases, caused for example by Nectria galligena;

Blight diseases such as:
Monilinia diseases, caused for example by Monilinia laxa;

Leaf blister or leaf curl diseases such as:
Exobasidium diseases caused for example by Exobasidium vexans
Taphrina diseases, caused for example by Taphrina deformans;

Decline diseases of wooden plants such as:
Esca diseases, caused for example by Phaemoniella clamydospora;
Eutypa dyeback, caused for example by Eutypa lata;
Ganoderma diseases caused for example by Ganoderma boninense;
Rigidoporus diseases caused for example by Rigidoporus lignosus

Diseases of Flowers and Seeds such as
Botrytis diseases caused for example by Botrytis cinerea;

Diseases of Tubers such as
Rhizoctonia diseases caused for example by Rhizoctonia solani;
Helminthosporium diseases caused for example by Helminthosporium solani;

Club root diseases such as
Plasmodiophora diseases, cause for example by Plamodiophora brassicae.

Diseases caused by Bacterial Organisms such as
Xanthomonas species for example Xanthomonas campestris pv. oryzae;
Pseudomonas species for example Pseudomonas syringae pv. lachrymans;
Erwinia species for example Erwinia amylovora.

The compounds according to the invention can also be used for the preparation of composition useful to curatively or preventively treat human or animal fungal diseases such as, for example, mycoses, dermatoses, trichophyton diseases and candidiases or diseases caused by Aspergillus spp., for example Aspergillus fumigatus.

As a further aspect, the present invention provides a product comprising a compounds (A) and (B), as herein defined, as a combined preparation for simultaneous, separate or sequential use in controlling the phytopathogenic fungi of plants, crops or seeds at a site.

The fungicide composition according to the invention can be prepared immediately before use by using a kit-of-parts for controlling, curatively or preventively, the phytopathogenic fungi of crops, such a kit-of-parts may comprise compound (A) and (B) intended to be combined or used simultaneously, separately or sequentially in controlling the phytopathogenic fungi of crops at a site. It is therefore a pack wherein the user finds all the ingredients for preparing the fungicide formulation which they wish to apply to the crops. These ingredients, which comprise in particular the active agents (A) and (B) and which are packaged separately, are provided in the form of a powder or in the form of a liquid which is concentrated to a greater or lesser degree. The user simply has to mix in the prescribed doses and to add the quantities of liquid, for example of water, necessary to obtain a formulation which is ready to use and which can be applied to the crops.

The various aspects of the invention will now be illustrated with reference to the following table 1 of compound examples, table 2 of intermediate examples and the following preparation or efficacy examples.

The following table 1 illustrates in a non-limiting manner examples of compounds of formula (I) according to the invention.

The following table 2 illustrates in a non-limiting manner examples of intermediates for the compounds of table 1.

In the following table, M+H (or M−H) means the molecular ion peak, plus or minus 1 a.m.u. (atomic mass unit) respectively, as observed in mass spectroscopy and M (ApcI+) means the molecular ion peak as it was found via positive atmospheric pressure chemical ionisation in mass spectroscopy

TABLE 1
(I)

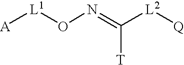

In table 1 we use the following abbreviations for specified claimed elements “A” of the generic structure (I) of the invention and we specify the
substituents of the element “Q” of the generic structure (I) as shown:

| A²-1 | A⁸-1 | A¹⁶-1 | A¹¹⁷-1 | Q |
|---|---|---|---|---|
| 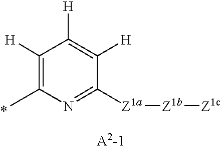 | 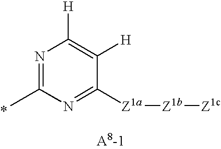 | 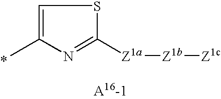 | 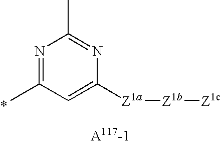 | 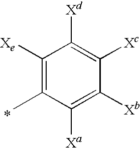 |

| | | | | | | | | | | | | | MW | |
|---|---|---|---|---|---|---|---|---|---|---|---|---|---|---|
| Example | A | Z^{1a} | Z^{1b} | Z^{1c} | L¹ | T | L² | X^{a} | X^{b} | X^{c} | X^{d} | X^{e} | Measure | logp |
| 1 | A²-1 | NH | carbonyl | CF₃ | CH₂ | 1-methyl-1H-tetrazol-5-yl | bond | F | H | CF₃ | H | H | 492 | 3.71 |
| 2 | A²-1 | NH | carbonyl | tert-butyl | CH₂ | 1-methyl-1H-tetrazol-5-yl | bond | F | H | CF₃ | H | H | 480 | 3.89 |
| 3 | A²-1 | NH | carbonyl | cyclopropyl | CH₂ | 1-methyl-1H-tetrazol-5-yl | bond | F | H | CF₃ | H | H | 464 | 3.37 |
| 4 | A²-1 | NH | carbonyl | propan-2-yl | CH₂ | 1-methyl-1H-tetrazol-5-yl | bond | F | H | CF₃ | H | H | 466 | 3.53 |
| 5 | A²-1 | NH | carbonyl | pentyloxy | CH₂ | 1-methyl-1H-tetrazol-5-yl | bond | CH₃ | F | H | H | H | 456 | 4.49 |
| 6 | A²-1 | NH | carbonyl | pentyl | CH₂ | 1-methyl-1H-tetrazol-5-yl | bond | H | CF₃ | Cl | H | H | 510 | 4.7 |
| 7 | A²-1 | NH | carbonyl | pentyloxy | CH₂ | 1-methyl-1H-tetrazol-5-yl | bond | H | F | H | CH₃ | H | 456 | 4.65 |
| 8 | A⁸-1 | NH | carbonyl | butoxy | CH₂ | 1-methyl-1H-tetrazol-5-yl | bond | H | CF₃ | CH₃ | H | H | 493 | 4.24 |
| 9 | A²-1 | NH | carbonyl | pentyloxy | CH₂ | 1-methyl-1H-tetrazol-5-yl | bond | F | H | H | CH₃ | H | 456 | 4.46 |
| 10 | A²-1 | NH | carbonyl | pentyloxy | CH₂ | 1-methyl-1H-tetrazol-5-yl | bond | H | F | H | CF₃ | H | 510 | 4.92 |
| 11 | A²-1 | NH | carbonyl | pentyloxy | CH₂ | 1-methyl-1H-tetrazol-5-yl | bond | H | F | CH₃ | H | H | 456 | 4.67 |
| 12 | A²-1 | NH | carbonyl | pentyloxy | CH₂ | 1-methyl-1H-tetrazol-5-yl | bond | F | H | CH₃ | H | H | 456 | 4.49 |
| 13 | A²-1 | NH | carbonyl | pentyloxy | CH₂ | 1-methyl-1H-tetrazol-5-yl | bond | F | CH₃ | H | H | H | 456 | 4.46 |
| 14 | A²-1 | NH | carbonyl | 2-methylpropyl | CH₂ | 1-methyl-1H-tetrazol-5-yl | bond | F | H | CF₃ | H | H | 480 | 3.83 |
| 15 | A²-1 | NH | carbonyl | propyl | CH₂ | 2-methyl-2H-tetrazol-5-yl | bond | H | phenoxy | F | H | H | 496 | 3.15 |
| 16 | A¹¹⁷-1 | NH | carbonyl | cyclopropyl | CH₂ | 2-methyl-2H-tetrazol-5-yl | bond | H | phenoxy | F | H | H | 503 | 2.72 |
| 17 | A²-1 | NH | carbonyl | pentyl | CH₂ | 2-methyl-2H-tetrazol-5-yl | bond | H | phenoxy | F | H | H | 524 | 4.13 |
| 18 | A¹¹⁷-1 | NH | carbonyl | tert-butyl | CH₂ | 1-methyl-1H-tetrazol-5-yl | bond | H | phenoxy | F | H | H | 519 | 3.18 |
| 19 | A²-1 | NH | carbonyl | cyclopropyl | CH₂ | 2-methyl-2H-tetrazol-5-yl | bond | H | phenoxy | F | H | H | 494 | 3.25 |
| 20 | A¹¹⁷-1 | NH | carbonyl | tert-butyl | CH₂ | 2-methyl-2H-tetrazol-5-yl | bond | H | phenoxy | F | H | H | 519 | 2.13 |
| 21 | A²-1 | NH | carbonyl | tert-butyl | CH₂ | 2-methyl-2H-tetrazol-5-yl | bond | H | phenoxy | F | H | H | 510 | 3.81 |
| 22 | A²-1 | NH | carbonyl | pentyloxy | CH₂ | 1-methyl-1H-tetrazol-5-yl | bond | H | CF₃ | CH₃ | H | H | 512 | 4.73 |
| 23 | A²-1 | NH | carbonyl | pentyloxy | CH₂ | 1-methyl-1H-tetrazol-5-yl | bond | F | H | CF₃ | H | H | 510 | 4.78 |
| 24 | A²-1 | NH | carbonyl | pentyloxy | CH₂ | 1-methyl-1H-tetrazol-5-yl | bond | F | H | CF₃ | H | H | 510 | 4.73 |
| 25 | A²-1 | NH | carbonyl | pentyloxy | CH₂ | 1-methyl-1H-tetrazol-5-yl | bond | F | CF₃ | H | H | H | 510 | 4.65 |
| 26 | A²-1 | NH | carbonyl | pentyl | CH₂ | 1-methyl-1H-tetrazol-5-yl | bond | F | H | CF₃ | H | H | 494 | 4.29 |
| 27 | A²-1 | NH | carbonyl | pentyl | CH₂ | 1-methyl-1H-tetrazol-5-yl | bond | F | CF₃ | H | H | H | 494 | 4.16 |
| 28 | A²-1 | NH | carbonyl | propan-2-yl | CH₂ | 1-methyl-1H-tetrazol-5-yl | bond | F | H | CF₃ | H | H | 466 | 3.48 |
| 29 | A²-1 | NH | carbonyl | pentyl | CH₂ | 1-methyl-1H-tetrazol-5-yl | bond | F | H | H | CH₃ | H | | 3.83 |
| 30 | A²-1 | NH | carbonyl | 2-methylpropyl | CH₂ | 1-methyl-1H-tetrazol-5-yl | bond | H | F | H | CF₃ | H | | 4.01 |
| 31 | A²-1 | NH | carbonyl | pentyl | CH₂ | 1-methyl-1H-tetrazol-5-yl | bond | CH₃ | H | H | CF₃ | H | | 4.01 |
| 32 | A²-1 | NH | carbonyl | 2-methylpropyl | CH₂ | 1-methyl-1H-tetrazol-5-yl | bond | H | F | CH₃ | H | H | | 3.71 |
| 33 | A²-1 | NH | carbonyl | 2-methylpropyl | CH₂ | 1-methyl-1H-tetrazol-5-yl | bond | F | CH₃ | H | H | H | | 3.51 |
| 34 | A²-1 | NH | carbonyl | 2-methylpropyl | CH₂ | 1-methyl-1H-tetrazol-5-yl | bond | CH₃ | H | CF₃O | H | H | | 4.16 |
| 35 | A²-1 | NH | carbonyl | 2-methylpropyl | CH₂ | 1-methyl-1H-tetrazol-5-yl | bond | Cl | H | H | CH₃ | H | | 3.73 |
| 36 | A⁸-1 | NH | carbonyl | pentyl | CH₂ | 1-methyl-1H-tetrazol-5-yl | bond | H | CF₃ | CH₃ | H | H | | |
| 37 | A²-1 | NH | carbonyl | 2-methylpropyl | CH₂ | 1-methyl-1H-tetrazol-5-yl | bond | H | CH₃ | F | H | H | | 3.67 |
| 38 | A²-1 | NH | carbonyl | 2-methylpropyl | CH₂ | 1-methyl-1H-tetrazol-5-yl | bond | F | H | CH₃ | H | H | | 3.53 |
| 39 | A²-1 | NH | carbonyl | pentyloxy | CH₂ | 1-methyl-1H-tetrazol-5-yl | bond | H | CH₃ | H | CH₃ | H | 452 | 4.86 |
| 40 | A²-1 | NH | carbonyl | 2-methylpropyl | CH₂ | 1-methyl-1H-tetrazol-5-yl | bond | Cl | H | CH₃ | H | H | | 3.73 |
| 41 | A²-1 | NH | carbonyl | 2-methylpropyl | CH₂ | 1-methyl-1H-tetrazol-5-yl | bond | H | F | CH₃ | H | H | | 3.71 |
| 42 | A²-1 | NH | carbonyl | pentyl | CH₂ | 1-methyl-1H-tetrazol-5-yl | bond | F | H | H | CH₃ | H | | 3.83 |
| 43 | A²-1 | NH | carbonyl | 2-methylpropyl | CH₂ | 1-methyl-1H-tetrazol-5-yl | bond | H | CF₃ | CH₃ | H | H | | 4.16 |
| 44 | A²-1 | NH | carbonyl | pentyloxy | CH₂ | 1-methyl-1H-tetrazol-5-yl | bond | CH₃ | CF₃ | H | H | H | | 4.78 |
| 45 | A²-1 | NH | carbonyl | pentyl | CH₂ | 1-methyl-1H-tetrazol-5-yl | bond | H | F | H | CF₃ | H | | 4.29 |
| 46 | A²-1 | NH | carbonyl | 2-methylpropyl | CH₂ | 1-methyl-1H-tetrazol-5-yl | bond | Cl | H | CH₃ | H | H | | 3.73 |
| 47 | A²-1 | NH | carbonyl | pentyl | CH₂ | 1-methyl-1H-tetrazol-5-yl | bond | H | Cl | CF₃ | H | H | | 4.7 |
| 48 | A²-1 | NH | carbonyl | 2-methylpropyl | CH₂ | 1-methyl-1H-tetrazol-5-yl | bond | F | H | H | CH₃ | H | | 3.53 |
| 49 | A²-1 | NH | carbonyl | pentyl | CH₂ | 1-methyl-1H-tetrazol-5-yl | bond | H | F | H | CF₃ | H | | 4.29 |
| 50 | A²-1 | NH | carbonyl | pentyl | CH₂ | 1-methyl-1H-tetrazol-5-yl | bond | H | CF₃ | CH₃ | H | H | | 4.41 |
| 51 | A²-1 | NH | carbonyl | 2-methylpropyl | CH₂ | 1-methyl-1H-tetrazol-5-yl | bond | Cl | H | CH₃ | H | H | | 3.53 |
| 52 | A⁸-1 | NH | carbonyl | pentyloxy | CH₂ | 1-methyl-1H-tetrazol-5-yl | bond | H | CF₃ | CH₃ | H | H | | 4.62 |
| 53 | A²-1 | NH | carbonyl | but-3-yn-1-yloxy | CH₂ | 1-methyl-1H-tetrazol-5-yl | bond | H | CH₃ | F | H | H | | 3.55 |
| 54 | A²-1 | NH | carbonyl | 2-cyclohexylethyl | CH₂ | 1-methyl-1H-tetrazol-5-yl | bond | H | CH₃ | F | H | H | | 4.92 |
| 55 | A²-1 | NH | carbonyl | but-3-en-1-yloxy | CH₂ | 1-methyl-1H-tetrazol-5-yl | bond | H | CH₃ | F | H | H | | 3.92 |
| 56 | A²-1 | NH | carbonyl | prop-2-en-1-yloxy | CH₂ | 1-methyl-1H-tetrazol-5-yl | bond | H | CH₃ | F | H | H | | 3.63 |
| 57 | A²-1 | NH | carbonyl | 2-phenoxyethyl | CH₂ | 1-methyl-1H-tetrazol-5-yl | bond | H | CH₃ | F | H | H | | 3.87 |
| 58 | A²-1 | NH | carbonyl | 2-cyclopentylethyl | CH₂ | 1-methyl-1H-tetrazol-5-yl | bond | H | CH₃ | F | H | H | | 4.56 |
| 59 | A²-1 | NH | carbonyl | 4-methoxybenzyl | CH₂ | 1-methyl-1H-tetrazol-5-yl | bond | H | CH₃ | F | H | H | | 3.68 |
| 60 | A²-1 | NH | carbonyl | 3-methoxybenzyl | CH₂ | 1-methyl-1H-tetrazol-5-yl | bond | H | CH₃ | F | H | H | | 3.72 |
| 61 | A²-1 | NH | carbonyl | 2-methoxybenzyl | CH₂ | 1-methyl-1H-tetrazol-5-yl | bond | H | CH₃ | F | H | H | | 3.9 |
| 62 | A²-1 | NH | carbonyl | 2-methylpropyl | CH₂ | 1-methyl-1H-tetrazol-5-yl | bond | Cl | H | H | CH₃ | H | | 3.53 |
| 63 | A²-1 | NH | carbonyl | 2-methylpropyl | CH₂ | 1-methyl-1H-tetrazol-5-yl | bond | Cl | H | H | CF₃ | H | | 3.96 |
| 64 | A²-1 | NH | carbonyl | 2-methylpropyl | CH₂ | 1-methyl-1H-tetrazol-5-yl | bond | CH₃ | H | H | CF₃ | H | | 3.94 |
| 65 | A²-1 | NH | carbonyl | 2-methylpropyl | CH₂ | 1-methyl-1H-tetrazol-5-yl | bond | H | F | H | CF₃ | H | | 3.89 |
| 66 | A²-1 | NH | carbonyl | 2-methylpropyl | CH₂ | 1-methyl-1H-tetrazol-5-yl | bond | H | CF₃ | F | H | H | | 3.94 |
| 67 | A²-1 | NH | carbonyl | 2-methylpropyl | CH₂ | 1-methyl-1H-tetrazol-5-yl | bond | CH₃ | H | H | CF₃ | H | | 3.62 |
| 68 | A²-1 | NH | carbonyl | 2-methylpropyl | CH₂ | 1-methyl-1H-tetrazol-5-yl | bond | H | CF₃ | CH₃ | H | H | | 4.01 |
| 69 | A²-1 | NH | carbonyl | 2-methylpropyl | CH₂ | 1-methyl-1H-tetrazol-5-yl | bond | H | CF₃ | CH₃ | H | H | | 4.14 |
| 70 | A²-1 | NH | carbonyl | 2-methylpropyl | CH₂ | 1-methyl-1H-tetrazol-5-yl | bond | H | CF₃ | Cl | H | H | | 4.26 |
| 71 | A²-1 | NH | carbonyl | 2-methylpropyl | CH₂ | 1-methyl-1H-tetrazol-5-yl | bond | H | Cl | CF₃ | H | H | | 4.29 |
| 72 | A²-1 | NH | carbonyl | 2-methylpropyl | CH₂ | 1-methyl-1H-tetrazol-5-yl | bond | H | Cl | CF₃ | H | H | | 4.29 |
| 73 | A²-1 | NH | carbonyl | 2-methylpropyl | CH₂ | 1-methyl-1H-tetrazol-5-yl | bond | CH₃ | F | H | H | H | | 3.53 |
| 74 | A²-1 | NH | carbonyl | pentyl | CH₂ | 1-methyl-1H-tetrazol-5-yl | bond | H | CH₃ | H | F | H | | 4.14 |
| 75 | A²-1 | NH | carbonyl | 2-methylpropyl | CH₂ | 1-methyl-1H-tetrazol-5-yl | bond | F | H | H | CH₃ | H | | 3.42 |
| 76 | A²-1 | NH | carbonyl | pentyloxy | CH₂ | 1-methyl-1H-tetrazol-5-yl | bond | H | F | CF₃ | H | H | 510 | 4.92 |
| 77 | A²-1 | NH | carbonyl | pentyl | CH₂ | 1-methyl-1H-tetrazol-5-yl | bond | H | CF₃ | Cl | H | H | | 4.67 |
| 78 | A²-1 | NH | carbonyl | pentyloxy | CH₂ | 1-methyl-1H-tetrazol-5-yl | bond | H | CF₃ | CH₃ | H | H | | 5.05 |
| 79 | A²-1 | NH | carbonyl | pentyloxy | CH₂ | 1-methyl-1H-tetrazol-5-yl | bond | H | Cl | CF₃ | H | H | | 5.23 |
| 80 | A²-1 | NH | carbonyl | pentyloxy | CH₂ | 1-methyl-1H-tetrazol-5-yl | bond | Cl | H | H | CF₃ | H | | 4.92 |
| 81 | A²-1 | NH | carbonyl | pentyloxy | CH₂ | 1-methyl-1H-tetrazol-5-yl | bond | H | CF₃ | CH₃ | H | H | | 5.08 |
| 82 | A²-1 | NH | carbonyl | pentyloxy | CH₂ | 1-methyl-1H-tetrazol-5-yl | bond | H | CF₃ | F | H | H | | 4.86 |
| 83 | A²-1 | NH | carbonyl | pentyloxy | CH₂ | 1-methyl-1H-tetrazol-5-yl | bond | Cl | H | H | CH₃ | H | | 4.54 |
| 84 | A²-1 | NH | carbonyl | pentyloxy | CH₂ | 1-methyl-1H-tetrazol-5-yl | bond | Cl | H | H | CH₃ | H | | 4.73 |
| 85 | A²-1 | NH | carbonyl | pentyloxy | CH₂ | 1-methyl-1H-tetrazol-5-yl | bond | Cl | H | CH₃ | H | H | 472 | 4.54 |
| 86 | A²-1 | NH | carbonyl | pentyloxy | CH₂ | 1-methyl-1H-tetrazol-5-yl | bond | H | Cl | CF₃ | H | H | | 5.2 |
| 87 | A²-1 | NH | carbonyl | pentyloxy | CH₂ | 1-methyl-1H-tetrazol-5-yl | bond | Cl | CF₃ | H | H | H | 526 | 4.81 |
| 88 | A²-1 | NH | carbonyl | pentyloxy | CH₂ | 1-methyl-1H-tetrazol-5-yl | bond | H | CF₃ | Cl | H | H | | 5.14 |
| 89 | A²-1 | NH | carbonyl | pentyl | CH₂ | 1-methyl-1H-tetrazol-5-yl | bond | H | CF₃ | CH₃ | H | H | 490 | 4.56 |
| 90 | A⁸-1 | NH | carbonyl | butyl | CH₂ | 1-methyl-1H-tetrazol-5-yl | bond | H | CF₃ | CH₃ | H | H | 477 | 3.94 |
| 91 | A²-1 | NH | carbonyl | pentyloxy | CH₂ | 1-methyl-1H-tetrazol-5-yl | bond | Cl | H | CH₃ | H | H | 472 | 4.7 |
| 92 | A²-1 | NH | carbonyl | pentyloxy | CH₂ | 1-methyl-1H-tetrazol-5-yl | bond | Cl | H | CH₃ | H | H | 472 | 4.54 |
| 93 | A²-1 | NH | carbonyl | pentyloxy | CH₂ | 1-methyl-1H-tetrazol-5-yl | bond | H | F | H | CH₃ | H | 456 | 4.65 |
| 94 | A²-1 | NH | carbonyl | pentyloxy | CH₂ | 1-methyl-1H-tetrazol-5-yl | bond | H | CH₃ | F | H | H | 456 | 4.65 |
| 95 | A²-1 | NH | carbonyl | pentyloxy | CH₂ | 1-methyl-1H-tetrazol-5-yl | bond | H | F | CH₃ | H | H | 456 | 4.67 |
| 96 | A²-1 | NH | carbonyl | pentyloxy | CH₂ | 1-methyl-1H-tetrazol-5-yl | bond | CH₃ | H | CF₃O | H | H | 522 | 4.89 |
| 97 | A²-1 | NH | carbonyl | pentyloxy | CH₂ | 1-methyl-1H-tetrazol-5-yl | bond | CH₃ | H | CF₃O | H | H | 522 | 5.11 |
| 98 | A²-1 | NH | carbonyl | pentyloxy | CH₂ | 1-methyl-1H-tetrazol-5-yl | bond | CH₃ | H | H | CF₃ | H | 512 | 4.36 |
| 99 | A²-1 | NH | carbonyl | pentyloxy | CH₂ | 1-methyl-1H-tetrazol-5-yl | bond | H | F | H | CF₃ | H | 516 | 4.62 |
| 100 | A²-1 | NH | carbonyl | pentyl | CH₂ | 1-methyl-1H-tetrazol-5-yl | bond | F | H | H | CH₃ | H | | 3.92 |
| 101 | A²-1 | NH | carbonyl | but-2-yn-1-yloxy | CH₂ | 1-methyl-1H-tetrazol-5-yl | bond | H | CH₃ | F | H | H | | 3.67 |
| 102 | A²-1 | NH | carbonyl | pentyl | CH₂ | 1-methyl-1H-tetrazol-5-yl | bond | H | F | CH₃ | H | H | | 4.21 |
| 103 | A²-1 | NH | carbonyl | pentyl | CH₂ | 1-methyl-1H-tetrazol-5-yl | bond | F | CH₃ | H | H | H | | 3.96 |
| 104 | A²-1 | NH | carbonyl | pentyl | CH₂ | 1-methyl-1H-tetrazol-5-yl | bond | H | CF₃ | F | H | H | | 4.36 |
| 105 | A²-1 | NH | carbonyl | pentyl | CH₂ | 1-methyl-1H-tetrazol-5-yl | bond | CH₃ | H | CF₃O | H | H | | 4.65 |
| 106 | A²-1 | NH | carbonyl | pentyl | CH₂ | 1-methyl-1H-tetrazol-5-yl | bond | Cl | H | H | CF₃ | H | | 4.39 |
| 107 | A²-1 | NH | carbonyl | pentyl | CH₂ | 1-methyl-1H-tetrazol-5-yl | bond | H | Cl | CF₃ | H | H | | 4.7 |
| 108 | A²-1 | NH | carbonyl | pentyl | CH₂ | 1-methyl-1H-tetrazol-5-yl | bond | CH₃ | H | CF₃O | H | H | | 4.39 |
| 109 | A²-1 | NH | carbonyl | pentyl | CH₂ | 1-methyl-1H-tetrazol-5-yl | bond | Cl | H | CH₃ | H | H | | 3.99 |
| 110 | A²-1 | NH | carbonyl | pentyloxy | CH₂ | 1-methyl-1H-tetrazol-5-yl | bond | CH₃ | H | H | CF₃ | H | | 4.84 |
| 111 | A²-1 | NH | carbonyl | pentyl | CH₂ | 1-methyl-1H-tetrazol-5-yl | bond | CH₃ | H | H | CF₃ | H | | 4.34 |
| 112 | A²-1 | NH | carbonyl | pentyl | CH₂ | 1-methyl-1H-tetrazol-5-yl | bond | CH₃ | F | H | H | H | | 3.96 |
| 113 | A²-1 | NH | carbonyl | pentyl | CH₂ | 1-methyl-1H-tetrazol-5-yl | bond | Cl | H | CH₃ | H | H | | 4.16 |
| 114 | A²-1 | NH | carbonyl | pentyl | CH₂ | 1-methyl-1H-tetrazol-5-yl | bond | H | F | CH₃ | H | H | | 4.16 |
| 115 | A²-1 | NH | carbonyl | pentyl | CH₂ | 1-methyl-1H-tetrazol-5-yl | bond | F | H | CH₃ | H | H | | 3.92 |
| 116 | A²-1 | NH | carbonyl | pentyl | CH₂ | 1-methyl-1H-tetrazol-5-yl | bond | Cl | H | H | CH₃ | H | | 3.99 |
| 117 | A²-1 | NH | carbonyl | pentyl | CH₂ | 1-methyl-1H-tetrazol-5-yl | bond | Cl | H | H | CH₃ | H | | 4.19 |
| 118 | A²-1 | NH | carbonyl | pentyl | CH₂ | 1-methyl-1H-tetrazol-5-yl | bond | H | CF₃ | CH₃ | H | H | | 4.56 |
| 119 | A²-1 | NH | carbonyl | pentyl | CH₂ | 1-methyl-1H-tetrazol-5-yl | bond | H | CH₃ | F | H | H | | 4.11 |
| 120 | A²-1 | NH | carbonyl | pentyl | CH₂ | 1-methyl-1H-tetrazol-5-yl | bond | H | F | H | CF₃ | H | | 4.41 |
| 121 | A²-1 | NH | carbonyl | pentyloxy | CH₂ | 1-methyl-1H-tetrazol-5-yl | bond | F | H | H | CH₃ | H | | 4.19 |
| 122 | A⁸-1 | NH | carbonyl | pentyloxy | CH₂ | 1-methyl-1H-tetrazol-5-yl | bond | H | CF₃ | CH₃ | H | H | | 4.59 |
| 123 | A²-1 | NH | carbonyl | pentyl | CH₂ | 1-methyl-1H-tetrazol-5-yl | bond | H | CH₃ | H | F | H | | 4.14 |
| 124 | A²-1 | NH | bond | H | CH₂ | 1-methyl-1H-tetrazol-5-yl | bond | CH₃ | H | H | CF₃ | H | 392 | 1.73 |
| 125 | A²-1 | NH | bond | H | CH₂ | 1-methyl-1H-tetrazol-5-yl | bond | H | CH₃ | H | CH₃ | H | 338 | 1.81 |
| 126 | A²-1 | NH | bond | H | CH₂ | 1-methyl-1H-tetrazol-5-yl | bond | Cl | H | CH₃ | H | H | 357 | 1.34 |
| 127 | A²-1 | NH | bond | H | CH₂ | 1-methyl-1H-tetrazol-5-yl | bond | H | F | H | CF₃ | H | 396 | 1.75 |
| 128 | A⁸-1 | NH | bond | H | CH₂ | 1-methyl-1H-tetrazol-5-yl | bond | H | CF₃ | CH₃ | H | H | 393 | 1.7 |
| 129 | A¹¹⁷-1 | NH | bond | H | CH₂ | 1-methyl-1H-tetrazol-5-yl | bond | H | phenoxy | F | H | H | 435 | 1.75 |
| 130 | A¹⁶-1 | NH | bond | H | CH₂ | 1-methyl-1H-tetrazol-5-yl | bond | CH₃ | H | H | CF₃ | H | 398 | 1.92 |
| 131 | A¹⁶-1 | NH | bond | H | CH₂ | 1-methyl-1H-tetrazol-5-yl | bond | H | F | H | CF₃ | H | 402 | 2.14 |
| 132 | A¹⁶-1 | NH | bond | H | CH₂ | 1-methyl-1H-tetrazol-5-yl | bond | F | H | H | CH₃ | H | 348 | 1.65 |
| 133 | A²-1 | NH | bond | H | CH₂ | 1-methyl-1H-tetrazol-5-yl | bond | Cl | H | H | CF₃ | H | 412 | 1.72 |
| 134 | A²-1 | NH | bond | H | CH₂ | 1-methyl-1H-tetrazol-5-yl | bond | H | Cl | CF₃ | H | H | 412 | 1.9 |
| 135 | A²-1 | NH | bond | H | CH₂ | 1-methyl-1H-tetrazol-5-yl | bond | H | F | H | CH₃ | H | 342 | 1.46 |
| 136 | A²-1 | NH | bond | H | CH₂ | 1-methyl-1H-tetrazol-5-yl | bond | F | H | H | CH₃ | H | 342 | 1.4 |
| 137 | A²-1 | NH | bond | H | CH₂ | 1-methyl-1H-tetrazol-5-yl | bond | H | F | CH₃ | H | H | 342 | 1.52 |
| 138 | A²-1 | NH | bond | H | CH₂ | 1-methyl-1H-tetrazol-5-yl | bond | F | CH₃ | H | H | H | 342 | 1.42 |
| 139 | A¹¹⁷-1 | NH | bond | H | CH₂ | 2-methyl-2H-tetrazol-5-yl | bond | H | phenoxy | F | H | H | 0 | 1.73 |
| 140 | A¹⁶-1 | NH | bond | H | CH₂ | 2-methyl-2H-tetrazol-5-yl | bond | H | phenoxy | F | H | H | 426 | 2.23 |
| 141 | A²-1 | NH | bond | H | CH₂ | 1-methyl-1H-tetrazol-5-yl | bond | F | H | CF₃ | H | H | 396 | 1.82 |
| 142 | A²-1 | NH | bond | H | CH₂ | 1-methyl-1H-tetrazol-5-yl | bond | F | H | CF₃ | H | H | 396 | 1.72 |
| 143 | A²-1 | NH | bond | H | CH₂ | 1-methyl-1H-tetrazol-5-yl | bond | F | CF₃ | H | H | H | 396 | 1.75 |
| 144 | A²-1 | NH | bond | H | CH₂ | 1-methyl-1H-tetrazol-5-yl | bond | F | CF₃ | H | H | H | 396 | 1.65 |

| Example | A | L¹ | T | L²Q | MW | Log p |
|---|---|---|---|---|---|---|
| 145 | 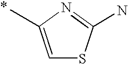 | CH₂ | 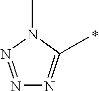 | 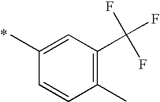 | 398 | 2.22 |
| 146 | 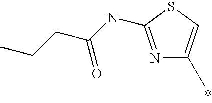 | CH₂ | 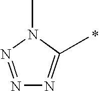 | 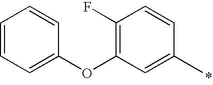 | 496 | 3.35 |
| 147 | 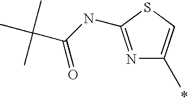 | CH₂ | 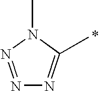 | 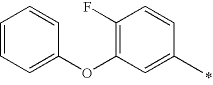 | 510 | 4.05 |
| 148 | 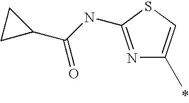 | CH₂ | 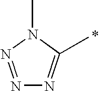 | 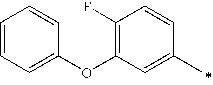 | 494 | 3.48 |
| 149 | 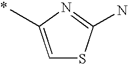 | CH₂ | 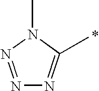 | 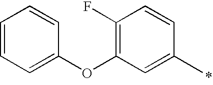 | 426 | 2.25 |
| 150 | 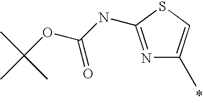 | CH₂ | 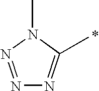 | 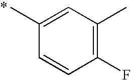 | | 3.89 |
| 151 | 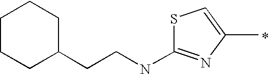 | CH₂ | 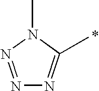 | 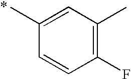 | | 4.36 |
| 152 | 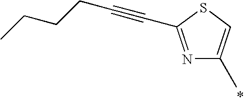 | CH₂ | 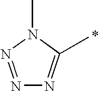 | 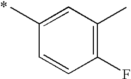 | | 4.78 |
| 153 | 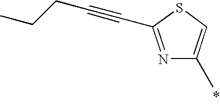 | CH₂ | 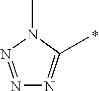 | 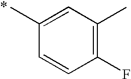 | | 4.36 |
| 154 | 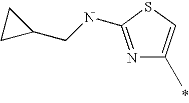 | CH₂ | 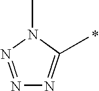 | 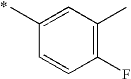 | | 2.71 |
| 155 | 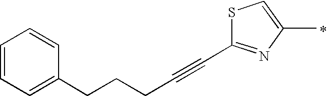 | CH₂ | 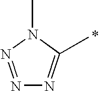 | 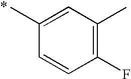 | | 5.32 |
| 156 | 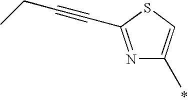 | CH₂ | 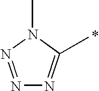 | 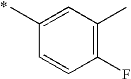 | | 4.09 |
| 157 | 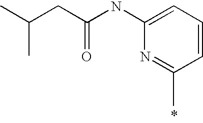 | CH₂ | 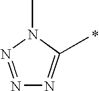 | 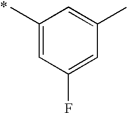 | | 3.71 |
| 158 | 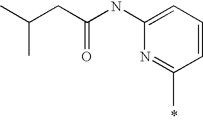 | CH₂ | 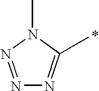 | 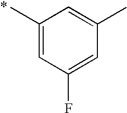 | | 3.76 |
| 159 | 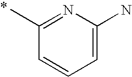 | CH₂ | 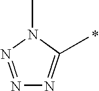 | 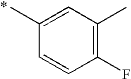 | | 1.50 |
| 160 | 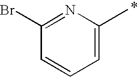 | CH₂ | 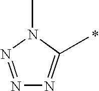 | 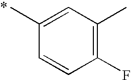 | | 3.65 |
| 161 | 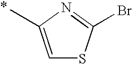 | CH₂ | 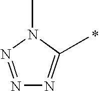 | 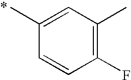 | | 3.57 |
| 162 | 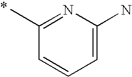 | CH₂ | 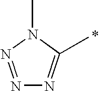 | 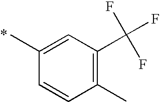 | | 2.10 |
| 163 | 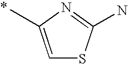 | CH₂ | 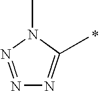 | 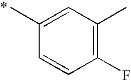 | | 1.75 |

Measurement of logP values was performed according EEC directive 79/831 Annex V.A8 by HPLC (High Performance Liquid Chromatography) on reversed phase columns with the following methods:
Measurement of LC-MS was done at pH 2.7 with 0.1% formic acid in water and with acetonitrile (contains 0.1% formic acid) as eluent with a linear gradient from 10% acetonitrle to 95% acetonitrile.
Calibration was done with not branched alkan2-ones (with 3 to 16 carbon atoms) with known logP-values (measurement of logP values using retention times with linear interpolation between successive alkanones) . . . lambda-maX-values were determined using UV-spectra from 200 nm to 400 nm and the peak values of the

TABLE 2
(I)

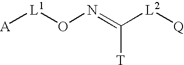

In table 2 we describe examples of intermediates for the compounds of table 1 and use
the following abbreviations for specified claimed elements “A” of the generic structure (I) of the
invention and we specify the substituents of the element “Q” of the generic structure (I) as shown:

| A²-1 | Q |
|---|---|
| 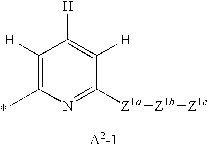 | 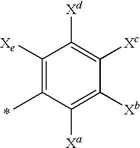 |

| | | | | | | | | | | | MW | |
|---|---|---|---|---|---|---|---|---|---|---|---|---|
| Ex- | | | | | | | X^{b} | X^{c} | | | Mea- | |
| ample | A | Z^{1a} + Z^{1b} + Z^{1c} | L¹ | T | L² | X^{a} | or X^{b +} X^{c} | | X^{d} | X^{e} | sure | logp |
| 1 | A²-1 | 1,3-dioxo- | CH₂ | 1-methyl-1H-tetrazol-5- | bond | H | CF₃ | F | H | H | 526 | 3.71 |
| | | 1,3-dihydro- | | yl | | | | | | | | |
| | | 2H-isoindol-2-yl | | | | | | | | | | |
| 2 | A²-1 | 1,3-dioxo- | CH₂ | 1-methyl-1H-tetrazol-5- | bond | F | H | H | CF₃ | H | 526 | 3.53 |
| | | 1,3-dihydro- | | yl | | | | | | | | |
| | | 2H-isoindol-2-yl | | | | | | | | | | |
| 3 | A²-1 | 1,3-dioxo- | CH₂ | 1-methyl-1H-tetrazol-5- | bond | H | F | H | CF₃ | H | 526 | 3.76 |
| | | 1,3-dihydro- | | yl | | | | | | | | |
| | | 2H-isoindol-2-yl | | | | | | | | | | |
| 4 | A²-1 | 1,3-dioxo- | CH₂ | 1-methyl-1H-tetrazol-5- | bond | CH₃ | H | H | CF₃ | H | 522 | 3.54 |
| | | 1,3-dihydro- | | yl | | | | | | | | |
| | | 2H-isoindol-2-yl | | | | | | | | | | |
| 5 | A²-1 | 1,3-dioxo- | CH₂ | 1-methyl-1H-tetrazol-5- | bond | F | H | H | CH₃ | H | 272 | 3.29 |
| | | 1,3-dihydro- | | yl | | | | | | | | |
| | | 2H-isoindol-2-yl | | | | | | | | | | |
| 6 | A²-1 | 1,3-dioxo- | CH₂ | 1-methyl-1H-tetrazol-5- | bond | H | CH₃ | H | CH₃ | H | 468 | 3.65 |
| | | 1,3-dihydro- | | yl | | | | | | | | |
| | | 2H-isoindol-2-yl | | | | | | | | | | |
| 7 | A²-1 | 1,3-dioxo- | CH₂ | 1-methyl-1H-tetrazol-5- | bond | Cl | H | CH₃ | H | H | 488 | 3.49 |
| | | 1,3-dihydro- | | yl | | | | | | | | |
| | | 2H-isoindol-2-yl | | | | | | | | | | |
| 8 | A²-1 | 1,3-dioxo- | CH₂ | 1-methyl-1H-tetrazol-5- | bond | F | H | CH₃ | H | H | 472 | 3.29 |
| | | 1,3-dihydro- | | yl | | | | | | | | |
| | | 2H-isoindol-2-yl | | | | | | | | | | |
| 9 | A²-1 | 1,3-dioxo- | CH₂ | 1-methyl-1H-tetrazol-5- | bond | H | F | CH₃ | H | H | 472 | 3.46 |
| | | 1,3-dihydro- | | yl | | | | | | | | |
| | | 2H-isoindol-2-yl | | | | | | | | | | |
| 10 | A²-1 | 1,3-dioxo- | CH₂ | 1-methyl-1H-tetrazol-5- | bond | H | Cl | CF₃ | H | H | 542 | 4.01 |
| | | 2H-isoindol-2-yl | | yl | | | | | | | | |
| | | 1,3-dihydro- | | | | | | | | | | |
| 11 | A2-1 | 1,3-dioxo- | CH₂ | 1-methyl-1H-tetrazol-5- | bond | CH₃ | F | H | H | H | 472 | 3.11 |
| | | 1,3-dihydro- | | yl | | | | | | | | |
| | | 2H-isoindol-2-yl | | | | | | | | | | |
| 12 | A²-1 | 1,3-dioxo- | CH₂ | 1-methyl-1H-tetrazol-5- | bond | H | F | H | CH₃ | H | 472 | 3.46 |
| | | 1,3-dihydro- | | yl | | | | | | | | |
| | | 2H-isoindol-2-yl | | | | | | | | | | |
| 13 | A²-1 | 1,3-dioxo- | CH₂ | 1-methyl-1H-tetrazol-5- | bond | H | CF₃ | Cl | H | H | 542 | 3.99 |
| | | 1,3-dihydro- | | yl | | | | | | | | |
| | | 2H-isoindol-2-yl | | | | | | | | | | |
| 14 | A²-1 | 1,3-dioxo- | CH₂ | 1-methyl-1H-tetrazol-5- | bond | H | CF₃ | CH₃ | H | H | 522 | 3.89 |
| | | 1,3-dihydro- | | yl | | | | | | | | |
| | | 2H-isoindol-2-yl | | | | | | | | | | |
| 15 | A²-1 | 1,3-dioxo- | CH₂ | 1-methyl-1H-tetrazol-5- | bond | F | CH₃ | H | H | H | 472 | 3.29 |
| | | 1,3-dihydro- | | yl | | | | | | | | |
| | | 2H-isoindol-2-yl | | | | | | | | | | |
| 16 | A²-1 | 1,3-dioxo- | CH₂ | 1-methyl-1H-tetrazol-5- | bond | H | F | CH₃ | H | H | 472 | 3.46 |
| | | 1,3-dihydro- | | yl | | | | | | | | |
| | | 2H-isoindol-2-yl | | | | | | | | | | |
| 17 | A²-1 | 1,3-dioxo- | CH₂ | 1-methyl-1H-tetrazol-5- | bond | H | F | CF₃ | H | H | 526 | 3.78 |
| | | 1,3-dihydro- | | yl | | | | | | | | |
| | | 2H-isoindol-2-yl | | | | | | | | | | |
| 18 | A²-1 | 1,3-dioxo- | CH₂ | 1-methyl-1H-tetrazol-5- | bond | Cl | H | H | CF₃ | H | 542 | 3.71 |
| | | 1,3-dihydro- | | yl | | | | | | | | |
| | | 2H-isoindol-2-yl | | | | | | | | | | |

| Ex- | | | | | | |
|---|---|---|---|---|---|---|
| ample | A | L¹ | T | L²Q | MW | Log p |
| 19 | 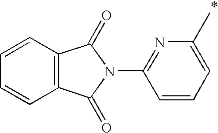 | CH₂ | 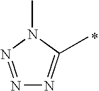 | 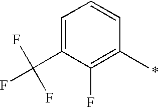 | 526 | 3.51 |
| 20 | 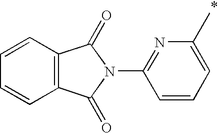 | CH₂ | 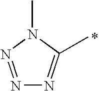 | 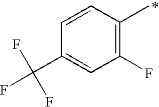 | 526 | 3.65 |

Measurement of logP values was performed according EEC directive 79/831 Annex V.A8 by HPLC (High Performance Liquid Chromatography) on reversed phase columns with the following methods:
Measurement of LC-MS was done at pH 2.7 with 0.1% formic acid in water and with acetonitrile (contains 0.1% formic acid) as eluent with a linear gradient from 10% acetonitrle to 95% acetonitrile.
Calibration was done with not branched alkan2-ones (with 3 to 16 carbon atoms) with known logP-values (measurement of logP values using retention times with linear interpolation between successive alkanones) . . . lambda-maX-values were determined using UV-spectra from 200 nm to 400 nm and the peak values of the chromatographic signals.

The following examples illustrate in a non-limiting manner the preparation and efficacy of the compounds of formula (I) according to the invention.

### EXAMPLE 1

### Preparation of 6-{[({[2-fluoro-3-(trifluoromethyl)phenyl](1-methyl-1H-tetrazol-5-yl)methylidene}amino)oxy]methyl}pyridin-2-amine (Compound 143) According to Process P1

Step 1

### Preparation of 2-fluoro-N-methoxy-N-methyl-3-(trifluoromethyl)benzamide

To a stirred solution of 2-fluoro-3-(trifluoromethyl)-benzoyl chloride (3.00 g, 12.24 mmol) in dichloromethane (20 mL), was added pyridine (3.14 g, 39.72 mmol) and N,O-dimethyl-hydroxylamine hydrochloride (1.93 g, 19.86 mmol) The reaction mixture was stirred at room temperature for 24 h. The reaction mixture was then poured into water (75 mL) and dichloromethane (25 mL). The organic layer was washed with water (50 mL), dried on MgSO₄, filtered and concentrated in vacuo to afford an oil. Purification on silica gel afforded 2-fluoro-N-methoxy-N-methyl-3-(trifluoromethyl)benzamide (2.72 g, 78%)

[M+1]=252

Step 2

### Preparation of 2-fluoro-3-(trifluoromethyl)phenyl](1-methyl-1H-tetrazol-5-yl)methanone

To a stirred solution of 1-methyl-1H-tetrazole (0.98 g, 11.67 mmol) in tetrahydrofuran (15 mL), was added 1,2-bis-(dimethylamino)-ethane (1.48 g, 12.73 mmol) and the reaction mixture was cooled to −75° C. A solution of BuLi (5.09 mL, 2.5 M in hexanes) was added dropwise to the reaction mixture over a period of 20 min After a further 8 min of stirring at −75° C., a solution of 2-fluoro-N-methoxy-N-methyl-3-(trifluoromethyl)benzamide (2.66 g, 10.61 mmol) in tetrahydrofuran (20 mL) was added over a period of 12 min After 4 h of stirring at −75° C., the reaction mixture was poured into a saturated aqueous solution of ammonium chloride (200 mL) and ice. The mixture was extracted with ethyl acetate (2×100 mL). The organic layer was washed with water (100 mL), dried on MgSO₄, filtered and concentrated in vacuo to afford an oil. Purification on silica gel afforded 2-fluoro-3-(trifluoromethyl)phenyl](1-methyl-1H-tetrazol-5-yl)methanone (0.61 g, 20%)

[M+1]=275

Step 3

### Preparation of 1-[2-fluoro-3-(trifluoromethyl)phenyl]-N-hydroxy-1-(1-methyl-1H-tetrazol-5-yl)methanimine

To a stirred solution of 2-fluoro-3-(trifluoromethyl)phenyl](1-methyl-1H-tetrazol-5-yl)methanone (534 mg, 1.95 mmol) in pyridine (15 mL), was added hydroxylamine hydrochloride (339 g, 4.87 mmol) and the reaction mixture was heated at 50° C. for 7.5 h and left overnight. The reaction mixture was concentrated in vacuo and taken up in ethyl acetate (50 mL). The solution was washed with water (50 mL), dried on MgSO₄, filtered and concentrated in vacuo to afford an oil. Purification on silica gel afforded 1-[2-fluoro-3-(trifluoromethyl)phenyl]-N-hydroxy-1-(1-methyl-1H-tetrazol-5-yl)methanimine (540 mg, 91%)

[M+1]=290

Step 4

### Preparation of 2-(6-{[({[2-fluoro-3-(trifluoromethyl)phenyl](1-methyl-1H-tetrazol-5-yl)methylidene}amino)oxy]methyl}pyridin-2-yl)-1H-isoindole-1,3(2H)-dione

To a stirred solution of 1-[2-fluoro-3-(trifluoromethyl)phenyl]-N-hydroxy-1-(1-methyl-1H-tetrazol-5-yl)methanimine (535 mg, 1.85 mmol) in acetonitrile (15 mL), was added 2-[6-(bromomethyl)pyridin-2-yl]-1H-isoindole-1,3(2H)-dione (645 mg, 2.03 mmol), cesium carbonate (1.26 g, 3.88 mmol), potassium iodide (31.0 mg, 0.18 mmol) and the reaction mixture was stirred at room temperature for 24 h. The reaction mixture was filtered, washed with ethyl acetate (30 mL) and the filtrate was concentrated in vacuo to afford an oil. Purification on silica gel afforded 2-(6-{[({[2-fluoro-3-(trifluoromethyl)phenyl](1-methyl-1H-tetrazol-5-yl)methylidene}amino)oxy]methyl}pyridin-2-yl)-1H-isoindole-1,3(2H)-dione (764 mg, 75%)

[M+1]=526

Step 5

### Preparation of 6-{[({[2-fluoro-3-(trifluoromethyl)phenyl](1-methyl-1H-tetrazol-5-yl)methylidene}amino)oxy]methyl}pyridin-2-amine (compound 143)

To a stirred solution 2-(6-{[({[2-fluoro-3-(trifluoromethyl)phenyl](1-methyl-1H-tetrazol-5-yl)methylidene}amino)oxy]methyl}pyridin-2-yl)-1H-isoindole-1,3(2H)-dione (759 mg, 1.44 mmol) in tetrahydrofuran (20 mL), was added hydrazine hydrate (361 mg, 7.22 mmol) and the reaction mixture was stirred at room temperature for 24 h. The reaction mixture was filtered, washed with ethyl acetate (50 mL) and the filtrate was concentrated in vacuo to afford an oil. Purification on silica gel afforded 6-{[({[2-fluoro-3-(trifluoromethyl)phenyl](1-methyl-1H-tetrazol-5-yl)methylidene}amino)oxy]methyl}pyridin-2-amine (297 mg, 50%)

[M+1]=396

### EXAMPLE 2

### Preparation of N-(6-{[({[2-fluoro-3-(trifluoromethyl)phenyl](1-methyl-1H-tetrazol-5-yl)methylidene}amino)oxy]methyl}pyridin-2-yl)hexanamide (Compound 27) According to Process P2

To a stirred solution of 6-{[({[2-fluoro-3-(trifluoromethyl)phenyl](1-methyl-1H-tetrazol-5-yl)methylidene}amino)oxy]methyl}pyridin-2-amine (131 mg, 0.33 mmol) in dioxane (10 mL), was added triethylamine (92.0 L, 0.66 mmol) and hexanoyl chloride (178 mg, 1.32 mmol) The reaction mixture was stirred at room temperature for 24 h. The reaction mixture was concentrated in vacuo, taken up in ethyl acetate (50 mL) and water (20 mL). The organic layer was dried on MgSO₄, filtered and concentrated in vacuo to afford an oil. Purification on silica gel afforded N-(6-{[({[2-fluoro-3-(trifluoromethyl)phenyl](1-methyl-1H-tetrazol-5-yl)methylidene}amino)oxy]methyl}pyridin-2-yl)hexanamide (540 mg, 91%)

[M+1]=494

### EXAMPLE 3

### Preparation of 2-{[({(1-methyl-1H-tetrazol-5-yl)[4-methyl-3-(trifluoromethyl)phenyl]methylidene}amino)oxy]methyl}pyrimidin-4-amine (Compound 128) According to Process P1

Step 1

### Preparation of N-methoxy-N,4-dimethyl-3-(trifluoromethyl)benzamide

To a stirred solution of 4-methyl-3-(trifluoromethyl)benzoyl chloride (5.00 g, 22.46 mmol) in dichloromethane (25 mL), was added triethylamine (5.23 g, 51.66 mmol) and N,O-dimethyl-hydroxylamine hydrochloride (2.41 g, 24.71 mmol) The reaction mixture was stirred at room temperature for 20 h. The reaction mixture was then extracted with water (75 mL) followed by 1N HCl. The organic layer was washed with water, dried over MgSO₄, filtered and concentrated in vacuo to afford N-methoxy-N,4-dimethyl-3-(trifluoromethyl)benzamide (5.38 g, 87%) as an oil which was used as such in the following step.

[M+1]=248

Step 2

### Preparation of (1-methyl-1H-1-tetrazol-5-yl)[4-methyl-3-(trifluoromethyl)phenyl]methanone

To a stirred solution of 1-methyl-1H-tetrazole (2.16 g, 25.73 mmol) in tetrahydrofuran (25 mL), was added at 0° C. dropwise 2-propylmagnesium chloride (2M in THF, 2.65 g, 25.73 mmol) and the reaction mixture was stirred for 15 min at 0° C. A solution of N-methoxy-N,4-dimethyl-3-(trifluoromethyl)benzamide (5.38 g, 21.44 mmol) in tetrahydrofuran (10 mL) was slowly added and the mixture was allowed to warm to room temperature. After further 1 h of stirring at room temperature 1N HCl (20 mL) and water (30 mL) were added. The mixture was extracted with ethyl acetate, the organic layer was washed with water, dried over MgSO₄, filtered and concentrated in vacuo. Purification of the crude material on silica gel afforded (1-methyl-1H-tetrazol-5-yl)[4-methyl-3-(trifluoromethyl)phenyl]methanone (3.13 g, 50%)

[M+1]=271

Step 3

### Preparation of N-hydroxy-1-(1-methyl-1H-tetrazol-5-yl)-1-[4-methyl-3-(trifluoromethyl)phenyl]methanimine

To a stirred solution of (1-methyl-1H-tetrazol-5-yl)[4-methyl-3-(trifluoromethyl)phenyl]methanone (3.1 g, 11.47 mmol) in pyridine (60 mL), was added hydroxylamine hydrochloride (1.99 g, 28.68 mmol) and the reaction mixture was heated at 50° C. for 19 h. The reaction mixture was concentrated in vacuo. Water (140 mL) was added, the precipitate formed was filtered and washed with diethylether (3×8 mL) to afford N-hydroxy-1-(1-methyl-1H-tetrazol-5-yl)-1-[4-methyl-3-(trifluoromethyl)phenyl]methanimine (2.57 g, 77%)

[M+1]=286

Step 4

### Preparation of 2-{[({(1-methyl-1H-tetrazol-5-yl)[4-methyl-3-(trifluoromethyl)phenyl]methylidene}amino)oxy]methyl}pyrimidin-4-amine (Compound 128)

To Si-TBD resin (2.147 g, 0.9 mmol/g) suspended in tetrahydrofuran (5 mL) was added N-hydroxy-1-(1-methyl-1H-tetrazol-5-yl)-1-[4-methyl-3-(trifluoromethyl)phenyl]methanimine (200 mg, 0.7 mmol) suspended in tetrahydrofuran (2.5 mL) and the suspension was shaked for 1 h at room temperature. After 1 h 2-(chloromethyl)pyrimidine (92 mg, 0.64 mmol) suspended in THF (2.5 mL) was added and shaking was continued at room temperature for 16 h. The reaction mixture was filtered, washed with tetrahydrofuran and the filtrate was concentrated in vacuo to afford 2-{[({(1-methyl-1H-tetrazol-5-yl)[4-methyl-3-(trifluoromethyl)phenyl]methylidene}amino)oxy]methyl}pyrimidin-4-amine (230 mg, 77%)

[M+1]=398

### EXAMPLE 4

### Preparation of butyl(2-{[({(1-methyl-1H-tetrazol-5-yl)[4-methyl-3-(trifluoromethyl)phenyl]methylidenelamino)oxy]methyl}pyrimidin-4-yl)carbamate (Compound 8) According to Process P2

To a stirred solution of 2-{[({(1-methyl-1H-tetrazol-5-yl)[4-methyl-3-(trifluoromethyl)phenyl]methylidene}amino)oxy]methyl}pyrimidin-4-amine (50 mg, 0.13 mmol) in dichloromethane (1.5 mL) were added pyridine (15 mg, 0.19 mmol) and after 15 min stirring n-Butyl chloroformate (35 mg, 0.26 mmol) dissolved in dichloromethane (0.5 mL). After stirring at room temperature for 16 h dichloromethane was added and the reaction mixture was extracted with saturated potassium carbonate solution. The organic layer was dried over MgSO₄, filtered and concentrated in vacuo. Purification on silica gel afforded butyl(2-{[({(1-methyl-1H-tetrazol-5-yl)[4-methyl-3-(trifluoromethyl)phenyl]methylidene}amino)oxy]methyl}pyrimidin-4-yl)carbamate (36 mg, 51%)

[M+1]=393

### EXAMPLE 5

### Preparation of 4-{[({((1-methyl-1H-tetrazol-5-yl)[4-methyl-3-(trifluoromethyl)phenyl]methylidene}amino)oxy]methyl}-1,3-thiazol-2-amine (Compound 145) According to Process P1

To Si-TBD resin (2.9 g, 0.9 mmol/g) suspended in tetrahydrofuran (5 mL) was added N-hydroxy-1-(1-methyl-1H-tetrazol-5-yl)-1-[4-methyl-3-(trifluoromethyl)phenyl]methanimine (200 mg, 0.7 mmol, prepared as described above) suspended in tetrahydrofuran (2.5 mL) and the suspension was shaked for 1 h at room temperature. After 1 h 4-(chloromethyl)-1,3-thiazol-2-amine hydrochloride (118 mg, 0.64 mmol) suspended in THF (2.5 mL) was added and shaking was continued at room temperature for 48 h. The reaction mixture was filtered, washed with tetrahydrofuran and the filtrate was concentrated in vacuo. Purification on silica gel afforded 4-{[({(1-methyl-1H -tetrazol-5-yl)[4-methyl-3-(trifluoromethyl)phenyl]methylidene}amino)oxy]methyl}-1,3-thiazol-2-amine (140 mg, 54%)

[M+1]=398

### EXAMPLE 6

### Preparation of pentyl(4-{[({(1-methyl-1H-tetrazol-5-yl)[4-methyl-3-(trifluoromethyl)phenyl]methylidene}amino)oxy]methyl}-1,3-thiazol-2-yl)carbamate (Compound 22)) According to Process P2

To a stirred solution of 4-{[({(1-methyl-1H-tetrazol-5-yl)[4-methyl-3-(trifluoromethyl)phenyl]methylidene}amino)oxy]methyl}-1,3-thiazol-2-amine (100 mg, 0.25 mmol) in dichloromethane (1.5 mL) were added pyridine (30 mg, 0.38 mmol) and after 15 min stirring pentyl carbonochloridoate (76 mg, 0.5 mmol) dissolved in dichloromethane (0.5 mL). After stirring at room temperature for 16 h dichloromethane was added and the reaction mixture was extracted with saturated potassium carbonate solution. The organic layer was dried over MgSO₄, filtered and concentrated in vacuo. Purification on silica gel afforded pentyl(4-{[({(1-methyl-1H-tetrazol-5-yl)[4-methyl-3-(trifluoromethyl)phenyl]methylidene}amino)oxy]methyl}-1,3-thiazol-2-yl)carbamate (36 mg, 51%)

[M+1]=512

### EXAMPLE 7

### Preparation of N-(cyclopropylmethyl)-4-{[({(4-fluoro-3-methylphenyl)(1-methyl-1H-tetrazol-5-yl)methylidene]amino}oxy)methyl]-1,3-thiazol-2-amine (Compound 154) According to Process P3

Step 1

### Preparation of tert-butyl {4-{[({(4-fluoro-3-methylphenyl)(1-methyl-1H-tetrazol-5-yl)methylidene]amino}oxy)methyl]-1,3-thiazol-2-yl}carbamate (Compound 150)

To a stirred solution of 1-(4-fluoro-3-methylphenyl)-N-hydroxy-1-(1-methyl-1H-tetrazol-5-yl)methanimine (470 mg, 2 mmol, prepared in analogy to compound 128) in acetonitrile (16 mL), was added tert-butyl[4-(chloromethyl)-1,3-thiazol-2-yl]carbamate (547 mg, 2.2 mmol), cesium carbonate (1.3 g, 4 mmol), potassium iodide (33.2 mg, 0.2 mmol) and the reaction mixture was stirred at room temperature for 48 h. After the addition of water the reaction mixture was extracted with ethyl acetate, the organic layer was dried over MgSO₄, filtered and concentrated in vacuo. Purification on silica gel afforded tert-butyl {4-[({[(4-fluoro-3-methylphenyl)(1-methyl-1H-tetrazol-5-yl)methylidene]amino}oxy)methyl]-1,3-thiazol-2-yl}carbamate (145 mg, 14%)

[M+1]=448

Step 2

### Preparation of N-(cyclopropylmethyl)-4-[({[(4-fluoro-3-methylphenyl)(1-methyl-1H-tetrazol-5-yl)methylidene]amino}oxy)methyl]-1,3-thiazol-2-amine (Compound 154)

To a stirred solution of tert-butyl {4-[({[(4-fluoro-3-methylphenyl)(1-methyl-1H-tetrazol-5-yl)methylidene]amino}oxy)methyl]-1,3-thiazol-2-yl}carbamate (145 mg, 0.32 mmol) in N,N-dimethylformamide (3 mL) were successively added sodium hydride (60 w/w, 14 mg, 0.35 mmol) and bromomethylcyclopropane (48 mg, 0.35 mmol). After stirring at room temperature for 16 h trifluoroacetic acid (1 mL) was added and the reaction mixture was stirred at 50° C. for further 8 h. After cooling water and saturated bicarbonate solution were added. The mixture was extracted with ethyl acetate. The organic layer was dried over MgSO₄, filtered and concentrated in vacuo. Purification on silica gel afforded N-(cyclopropylmethyl)-4-[({[(4-fluoro-3-methylphenyl)(1-methyl-1H-tetrazol-5-yl)methylidene]amino}oxy)methyl]-1,3-thiazol-2-amine (22 mg, 15%)

[M+1]=402

### EXAMPLE 8

### Preparation of 1-(4-fluoro-3-methylphenyl)-1-(1-methyl-1H-tetrazol-5-yl)-N-{[2-(pent-1-yn-1-yl)-1,3-thiazol-4-yl]methoxy}methanimine (Compound 153) According to Process P5

Step 1

### Preparation of N-[(2-bromo-1,3-thiazol-4-yl)methoxy]-1-(4-fluoro-3-methylphenyl)-1-(1-methyl-1H-tetrazol-5-yl)methanimine (Compound 161)

To a stirred solution of 4-[({[(4-fluoro-3-methylphenyl)(1-methyl-1H-tetrazol-5-yl)methylidene]amino}oxy)methyl]-1,3-thiazol-2-amine (250 mg, 0.72 mmol, prepared in analogy to compound LEGS474) in acetonitrile (10 mL), were added sodium bromide (222 mg, 2.2 mmol), copper(I) bromide (103 mg, 0.72 mmol), tert-butyl nitrite (74 mg, 0.72 mmol) and the reaction mixture was stirred at 70° C. for 15 minutes. After cooling and evaporation of the solvent in vacuo water (25 mL) was added and the mixture was extracted with dichloromethane (25 mL). The organic layer was dried over MgSO₄, filtered and concentrated in vacuo to afford N-[(2-bromo -1,3-thiazol-4-yl)methoxy]-1-(4-fluoro-3-methylphenyl)-1-(1-methyl-1H-tetrazol-5-yl)methanimine (300 mg, 91%)

[M+1]=412

Step 2

### Preparation of 1-(4-fluoro-3-methylphenyl)-1-(1-methyl-1H-tetrazol-5-yl)-N-{[2-(pent-1-yn-1-yl)-1,3-thiazol-4-yl]methoxy}methanimine (Compound 153)

A solution of N-[(2-bromo-1,3-thiazol-4-yl)methoxy]-1-(4-fluoro-3-methylphenyl)-1-(1-methyl-1H-tetrazol-5-yl)methanimine (50 mg, 0.12 mmol), 1-pentyne (33 mg, 0.48 mmol), copper(I) iodide (7 mg, 0.04 mmol), tetrakis(triphenylphosphine)palladium(0) (42 mg, 0.04 mmol), N,N-diisopropylethylamine (82 mg, 0.6 mmol) in tetrahydrofuran (2 mL) was stirred at room temperature for 16 h. After the addition of ethyl acetate (2 mL) the suspension was filtered over a pad of Celite and the filtrate was concentrated in vacuo. Purification on silica gel afforded 1-(4-fluoro-3-methylphenyl)-1-(1-methyl-1H-tetrazol-5-yl)-N-{[2-(pent-1-yn-1yl)-1,3-thiazol-4-yl]methoxy}methanimine (12 mg, 22%)

[M+1]=399

### BIOLOGICAL EXAMPLES

### EXAMPLE A

### Phytophthora Test (Tomato)/Preventive

Solvent: 49 parts by weight of N,N-Dimethylformamide

Emulsifier: 1 part by weight of Alkylarylpolyglycolether

To produce a suitable preparation of active compound, 1 part by weight of active compound is mixed with the stated amounts of solvent and emulsifier, and the concentrate is diluted with water to the desired concentration.

To test for preventive activity, young plants are sprayed with the preparation of active compound at the stated rate of application. One day after this treatment, the plants are inoculated with an aqueous spore suspension of Phytophthora infestans. The plants remain for one day in an incubation cabinet at approximately 22° C. and a relative atmospheric humidity of 100%. Then the plants are placed in an incubation cabinet at approximately 20° C. and a relative atmospheric humidity of 96%.

The test is evaluated 7 days after the inoculation. 0% means an efficacy which corresponds to that of the control, while an efficacy of 100% means that no disease is observed.

In this test the examples Nr. 5, 6, 7, 8, 10, 11, 12, 13, 24, 25, 27, 33, 36, 37, 39, 41, 43, 50, 66, 68, 69, 70, 73, 74, 77, 78, 81, 82, 88, 89, 93, 94, 95, 102, 103, 104, 112, 114, 115, 118, 119, 120, 122, 123 and 125 of table 1 showed efficacy of 70% or even higher at a concentration of 500 ppm of active ingredient:

### EXAMPLE B

### Plasmopara Test (Grapevines)/Protective

Solvent: 24.5 parts by weight of acetone
24.5 parts by weight of dimethylacetamide

Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated rate of application. After the spray coating has dried on, the plants are inoculated with an aqueous spore suspension of Plasmopara viticola and then remain for 1 day in an incubation cabinet at approximately 20° C. and a relative atmospheric humidity of 100%. The plant is subsequently placed for 4 days in a greenhouse at approximately 21° C. and a relative atmospheric humidity of approximately 90%. The plants are then misted and placed for 1 day in an incubation cabinet.

The test is evaluated 6 days after the inoculation. 0% means an efficacy which corresponds to that of the control, while an efficacy of 100% means that no disease is observed.

In this test the examples 5, 6, 7, 10, 11, 13, 37, 41, 43, 66, 70, 73, 77, 78, 81, 82, 88, 89, 93, 94, 95, 102, 103, 104, 112, 114, 115, 118, 119, 122 and 123 of table 1 showed efficacy of 70% or even higher at a concentration of 100 ppm of active ingredient.

### EXAMPLE C

### Pythium Test (Cotton)/Seed Treatment

The test is performed in the greenhouse. Cotton seeds of the variety Schmitz-Laux were sown in 6*6 cm pots containing a mix of steamed field soil and sand (1:1), 2 replicates were made. The active ingredient was solved in a solvent and seed treatment was performed with a lab equipment.

Infected perlite was scattered between cotton seeds. Seeds were then covered by 3 cm of LECA (light expanded clay aggregate).

Pots were incubated in the greenhouse 7 days at 20° C. and 80% relative humidity.

Assessment consisted of counting of emerged seedlings. 0% means an efficacy which corresponds to that of the control, while an efficacy of 100% means that all seedlings have emerged.

In this test the examples 94 and 95 of table 1 showed efficacy of 70% or even higher at a dose rate of 50 g of active ingredient/100 kg seed.

1. A hydroximoyl-tetrazole derivative of formula (I) 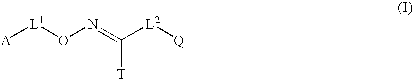
wherein
T represents a substituted or non-substituted tetrazolyl group;
L¹ represents a direct bond or a divalent group selected in the list consisting of
—(CR¹R²)ₙ— —(CR¹R²)ₘ—C(═O)—(CR¹R²)ₚ—
—(CR¹R²)ₘ—(CR¹═CR²)—(CR¹R²)ₚ— —(CR¹R²)ₘ—C(═O)—(CR¹R²)ₚ
—(CR¹R²)ₘ—C≡C—(CR¹R²)ₚ— —(CR¹R²)ₘ—O—C(═O)—(CR¹R²)ₚ—
—(CR¹R²)ₘ—O—(CR¹R²)ₚ— —(CR¹R²)ₘ—C(═O)—NH—(CR¹R²)ₚ—
—(CR¹R²)ₘ—NH—(CR¹R²)ₚ— —(CR¹R²)ₘ—NH—C(═O)—(CR¹R²)ₚ—
wherein
n represents 1, 2, 3 or 4;
m and p independently represent 0, 1, 2 or 3;
L² represents a direct bond or a divalent group selected in the list consisting of
—(CR³R⁴)_{q}— —(CR³R⁴)ₐ—C(═O)—(CR³R⁴)_{b}—
—(CR³R⁴)ₐ—(CR³═CR⁴)—(CR³R⁴)_{b}— —(CR³R⁴)ₐ—C(═O)—O—(CR³R⁴)_{b}
—(CR³R⁴)ₐ—C≡C—(CR³R⁴)_{b}— —(CR³R⁴)ₐ—O—C(═O)—(CR³R⁴)_{b}—
—(CR³R⁴)ₐ—O—(CR³R⁴)_{b}— —(CR³R⁴)ₐ—C(═O)—NH—(CR³R⁴)_{b}—
—(CR³R⁴)ₐ—NH—(CR³R⁴)_{b}— —(CR³R⁴)ₐ—NH—C(═O)—(CR³R⁴)_{b}—
wherein
q represents 1, 2, 3 or 4;
a and b independently represent 0, 1, 2 or 3;
A is selected in the list consisting of A¹ to A¹¹⁶ 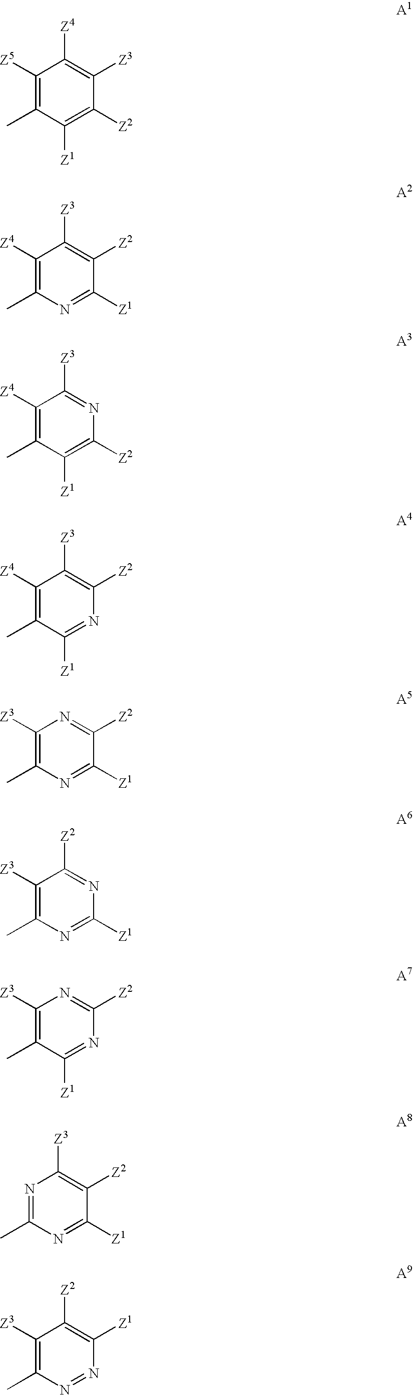 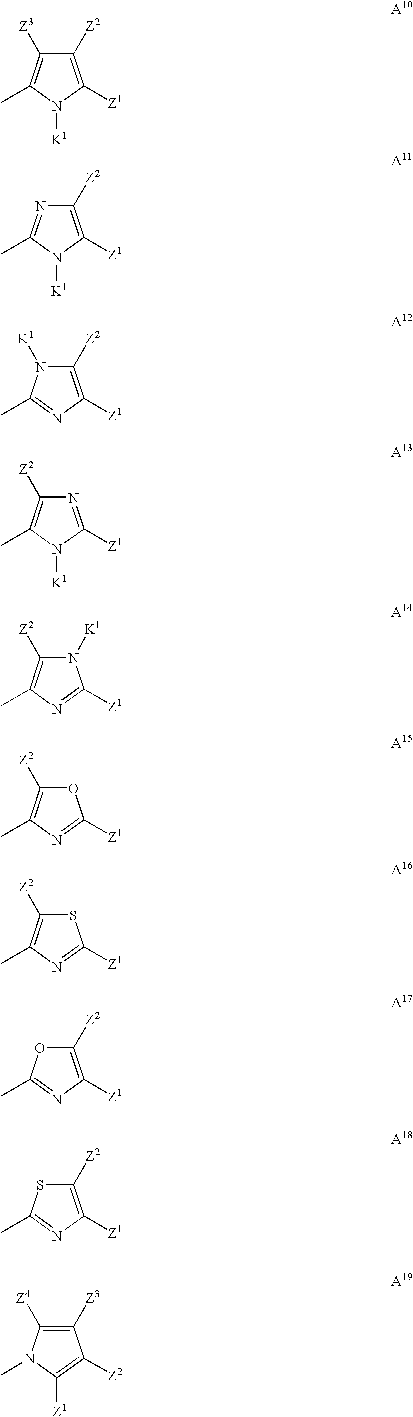 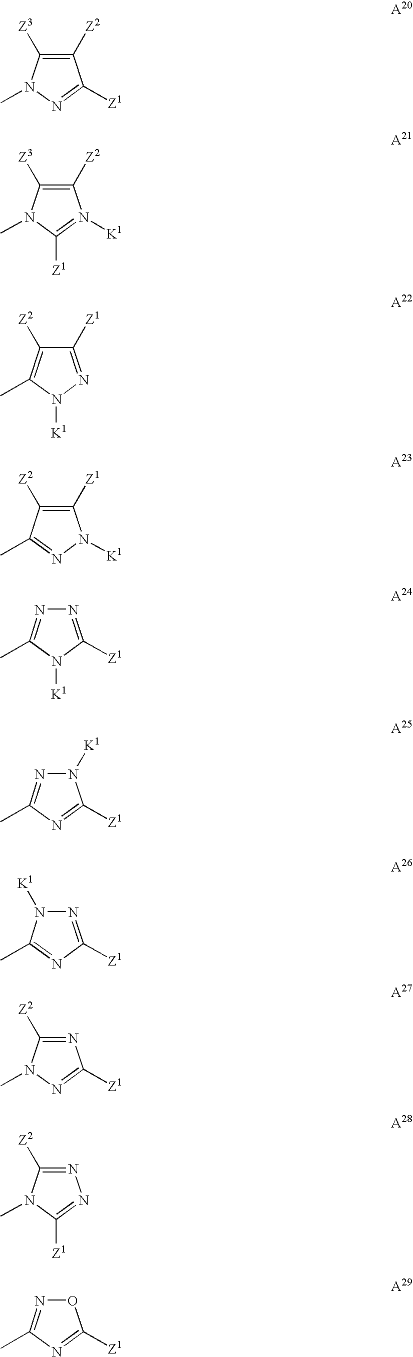 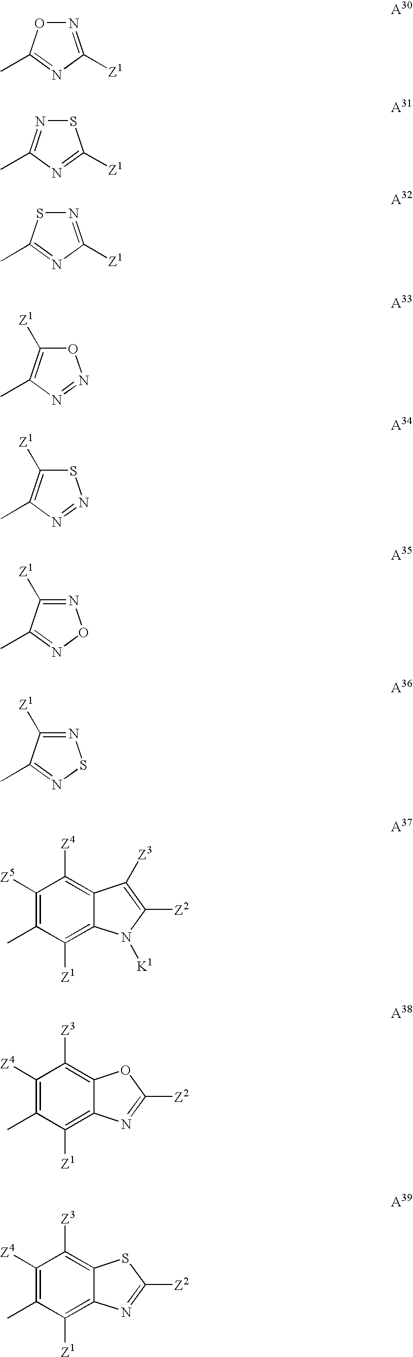 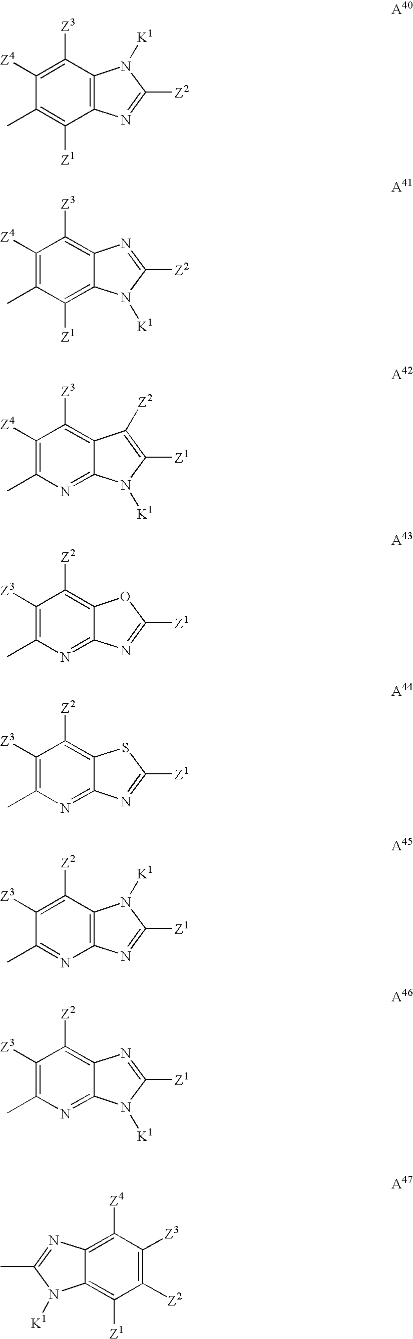 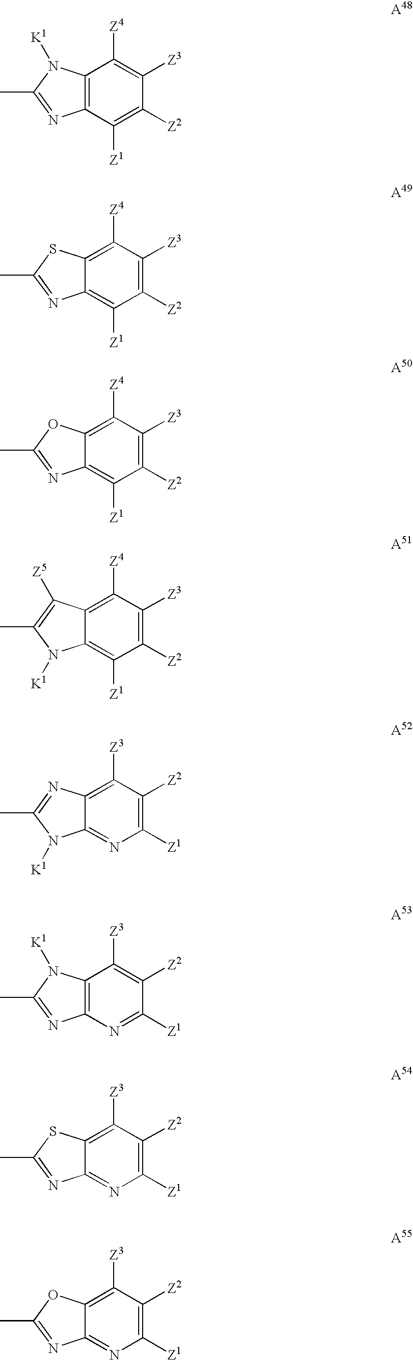 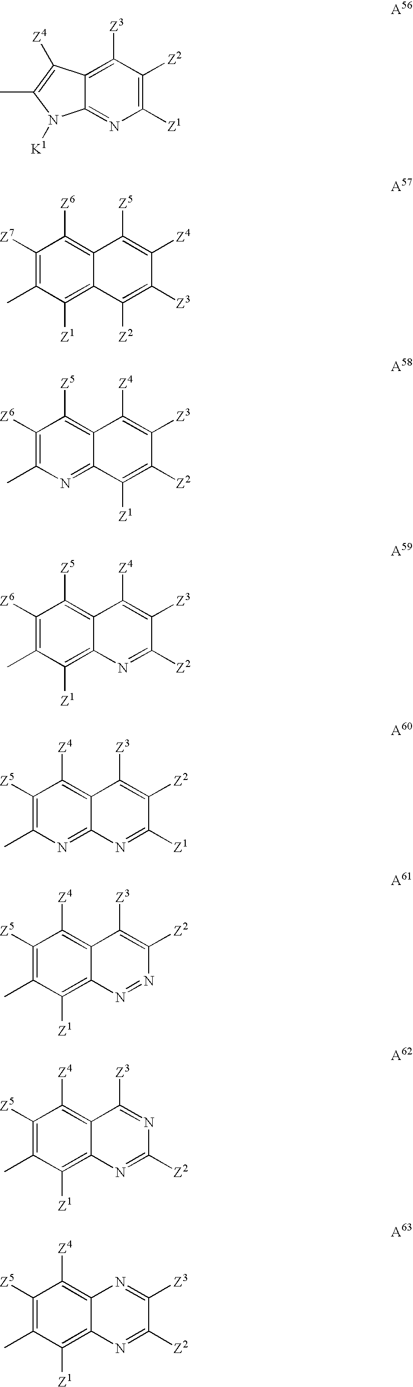 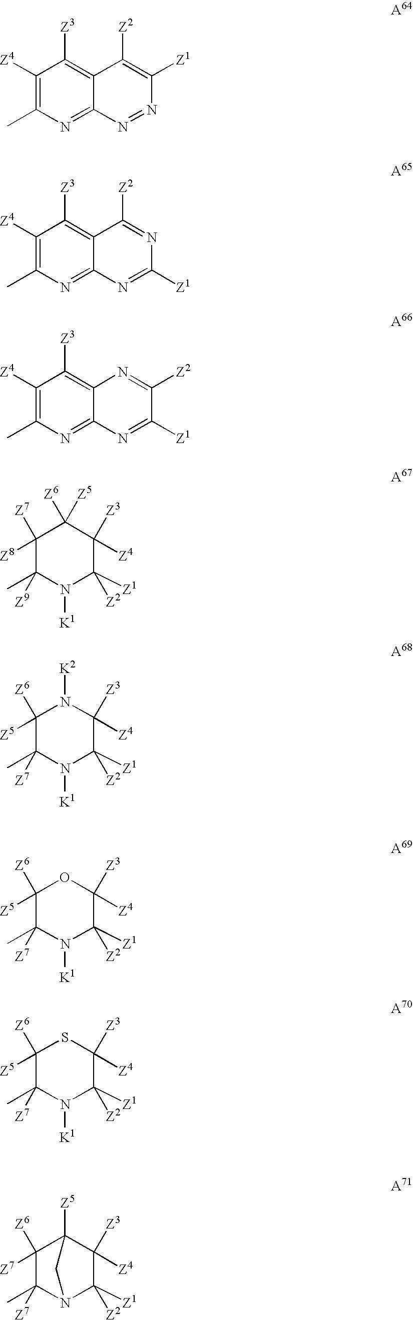 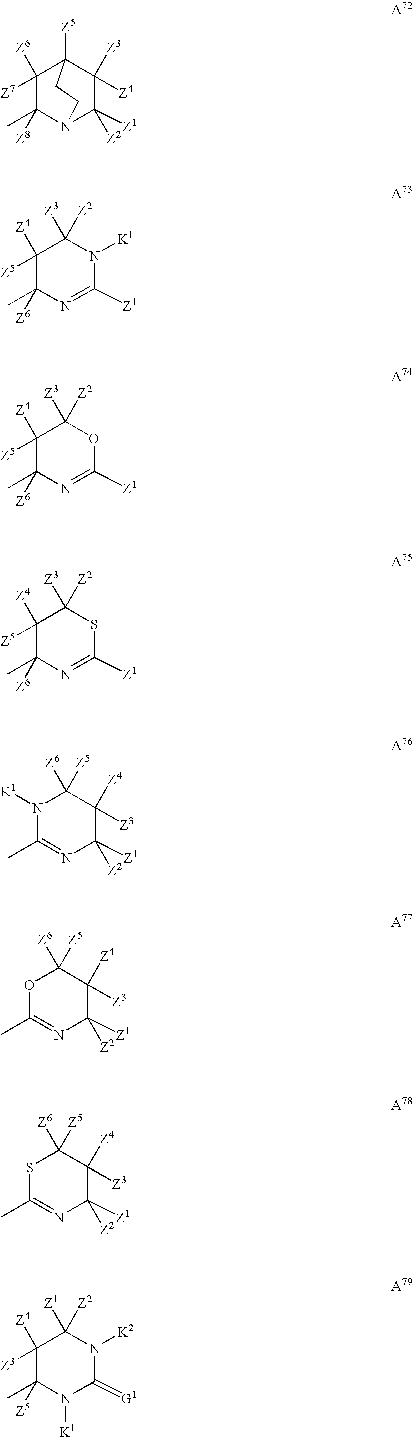 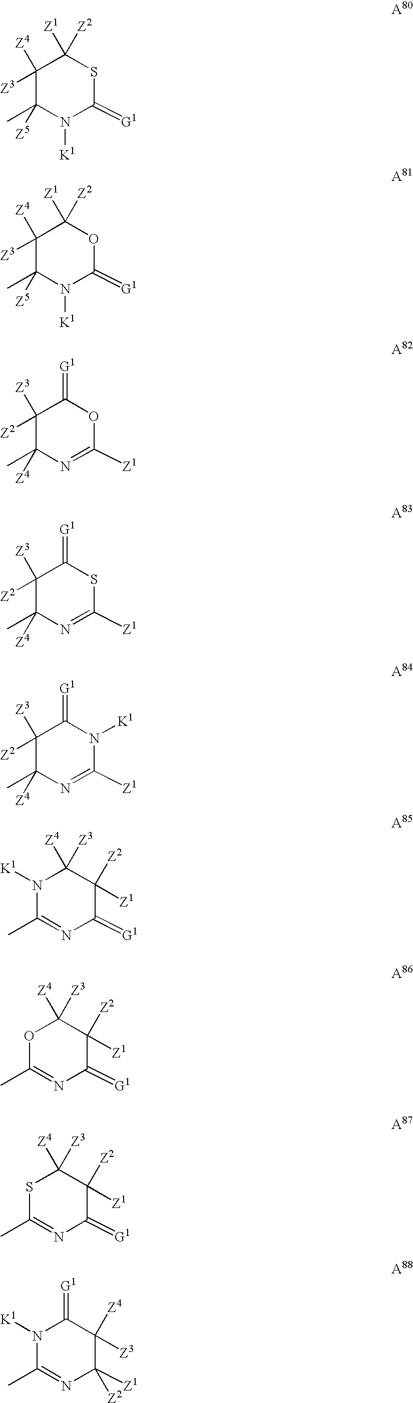 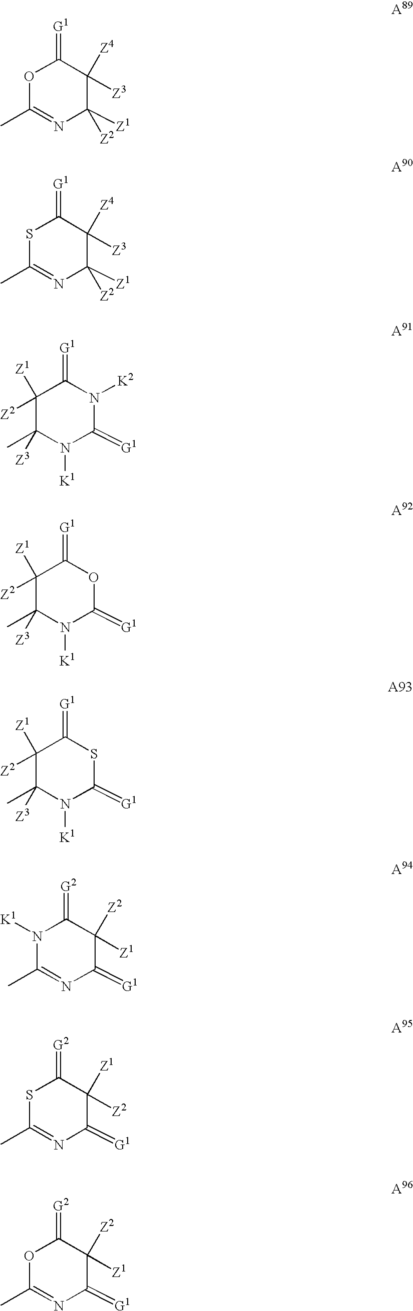 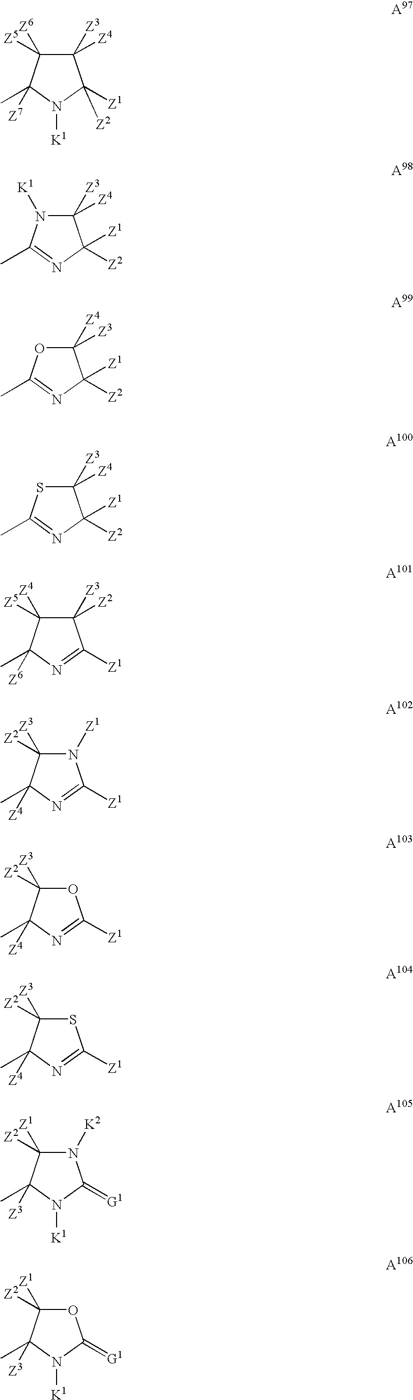 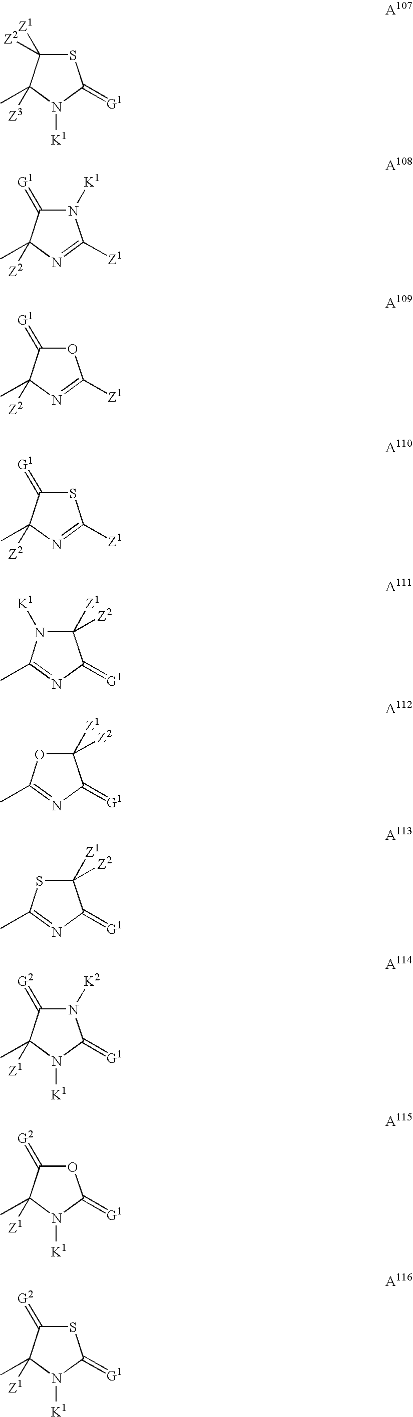
wherein
Z¹, Z², Z³, Z⁵, Z⁶, Z⁸ and Z⁹ independently represent a hydrogen atom, a halogen atom, a nitro group, a hydroxy group, a cyano group, an amino group, a sulphenyl group, a formyl group, a substituted or non-substituted carbaldehyde O—(C₁-C₈-alkyl)oxime, a formyloxy group, a formylamino group, a carbamoyl group, a N-hydroxycarbamoyl group, a pentafluoro-□⁶-sulphenyl group, a formylamino group, substituted or non-substituted C₁-C₈-alkoxyamino group, substituted or non-substituted N—C₁-C₈-alkyl-(C₁-C₈-alkoxy)-amino group, substituted or non-substituted (C₁-C₈-alkylamino)-amino group, substituted or non-substituted N—C₁-C₈-alkyl-(C₁-C₈-alkylamino)-amino group, a substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C₃-C₈-cycloalkyl, substituted or non-substituted tri(C₁-C₈-alkyl)silyl-C₃-C₈-cycloalkyl, substituted or non-substituted C₁-C₈-halogenoalkyl having 1 to 5 halogen atoms, substituted or non-substituted C₃-C₈-halogenocycloalkyl having 1 to 5 halogen atoms, a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted (C₁-C₈-alkoxycarbonyl)amino, substituted or non-substituted (C₃-C₈-cycloalkoxycarbonyl)amino, substituted or non-substituted C₁-C₈-halogenoalkoxy having 1 to 5 halogen atoms, substituted or non-substituted C₁-C₈-alkylsulphenyl, substituted or non-substituted C₁-C₈-halogenoalkylsulphenyl having 1 to 5 halogen atoms, substituted or non-substituted C₂-C₈-alkenyloxy, substituted or non-substituted (C₂-C₈-alkenyloxycarbonyl)amino, substituted or non-substituted C₂-C₈-halogenoalkenyloxy having 1 to 5 halogen atoms, substituted or non-substituted C₃-C₈-alkynyloxy, substituted or non-substituted (C₃-C₈-alkynyloxycarbonyl)amino, substituted or non-substituted C₃-C₈-halogenoalkynyloxy having 1 to 5 halogen atoms, substituted or non-substituted C₁-C₈-alkylcarbonyl, substituted or non-substituted N—(C₁-C₈-alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈-alkylcarbamoylamino, substituted or non-substituted C₁-C₈-halogenoalkylcarbamoylamino having 1 to 5 halogen atoms, substituted or non-substituted di-C₁-C₈-alkylcarbamoylamino, substituted or non-substituted di-C₁-C₈-halogenoalkylcarbamoylamino having 1 to 5 halogen atoms, substituted or non-substituted N—C₁-C₈-alkyl-(C₁-C₈-alkylcarbamoyl)amino, substituted or non-substituted N—C₁-C₈-alkyl-(C₁-C₈-halogenoalkylcarbamoyl)amino having 1 to 5 halogen atoms, substituted or non-substituted N—C₁-C₈-alkyl-(di-C₁-C₈-alkylcarbamoyl)amino, substituted or non-substituted N—C₁-C₈-alkyl-(di-C₁-C₈-halogenoalkylcarbamoyl)amino having 1 to 5 halogen atoms, substituted or non-substituted C₁-C₈-alkylaminocarbonyloxy, substituted or non-substituted di-C₁-C₈-alkylaminocarbonyloxy, substituted or non-substituted C₁-C₈-alkylcarbamothioyl, substituted or non-substituted di-C₁-C₈-alkylcarbamothioyl, substituted or non-substituted N—C₁-C₈-alkyloxycarbamothioyll, substituted or non-substituted C₁-C₈-alkoxycarbamothioyl, substituted or non-substituted N—C₁-C₈-alkyl-C₁-C₈-alkoxycarbamothioyll, substituted or non-substituted C₁-C₈-alkylthioylamino, substituted or non-substituted (C₁-C₈-alkoxythiocarbonyl)amino, substituted or non-substituted C₁-C₈-halogenoalkylthioylamino having 1 to 5 halogen atoms, substituted or non-substituted (C₁-C₈-alkyl-carbamothioyl)-oxy, substituted or non-substituted substituted or non-substituted (di-C₁-C₈-alkyl-carbamothioyl)-oxy, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted C₁-C₈-alkylaminosulfamoyl, substituted or non-substituted di-C₁-C₈-alkylaminosulfamoyl, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, substituted or non-substituted (benzyloxyimino)-C₁-C₆-alkyl, substituted or non-substituted benzyloxy, substituted or non-substituted benzylsulphenyl, substituted or non-substituted benzylamino, substituted or non-substituted phenoxy, substituted or non-substituted phenylsulphenyl, substituted or non-substituted phenylamino, substituted or non-substituted aryl, substituted or non-substituted (arylcarbonyl)amino, substituted or non-substituted (heterocyclylcarbonyl)amino substituted or non-substituted aryl-[C₁-C₈]-alkyl, substituted or non-substituted tri(C₁-C₈-alkyl)-silyloxy, substituted or non-substituted C₁-C₈-alkylsulfenylamino, substituted or non-substituted C₁-C₈-halogenoalkylsulphinylamino having 1 to 5 halogen atoms, substituted or non-substituted C₁-C₈-alkylsulphonylamino, substituted or non-substituted C₁-C₈-halogenoalkylsulphonylamino having 1 to 5 halogen atoms, substituted or non-substituted C₁-C₈-alkoxysulphonylamino, substituted or non-substituted C₁-C₈-halogenoxysulphonylamino having 1 to 5 halogen atoms, substituted or non-substituted tri(C₁-C₈-alkyl)-silyl, substituted or non-substituted (C₁-C₆-alkylideneamino)oxy, substituted or non-substituted (C₁-C₆-alkenylideneamino)oxy, substituted or non-substituted (C₁-C₆-alkynylideneamino)oxy, substituted or non-substituted (benzylideneamino)oxy; provided that when A represents A² or A¹⁶ and when Z¹, Z², Z³, Z⁴, Z⁵, Z⁶, Z⁷, Z⁸ and Z⁹ independently represent a substituted C₁-C₈-alkylamino or substituted benzylamino, then the substituent is chosen in the list consisting of a pentafluoro-λ⁶-sulphenyl group, a substituted or non-substituted carbaldehyde O—(C₁-C₈-alkyl)oxime, a formylamino group, a (hydroxyimino)-C₁-C₆-alkyl group, a tri(C₁-C₈-alkyl)silyl-C₁-C₈-alkyl, C₃-C₈-cycloalkyl, tri(C₁-C₈-alkyl)silyl-C₁-C₈-cycloalkyl, a C₁-C₈-halogenocycloalkyl having 1 to 5 halogen atoms, C₃-C₈-cycloalkoxy, a C₂-C₈-halogenoalkenyloxy having 1 to 5 halogen atoms, a C₃-C₈-halogenoalkynyloxy having 1 to 5 halogen atoms, a (C₁-C₆-alkoxyimino)-C₁-C₆-alkyl, a (C₁-C₆-alkenyloxyimino)-C₁-C₆-alkyl, a (C₁-C₆-alkynyloxyimino)-C₁-C₆-alkyl, and (benzyloxyimino)-C₁-C₆-alkyl.
K¹and K² independently represent a hydrogen atom, a formyl group, a substituted or non-substituted carbaldehyde O—(C₁-C₈-alkyl)oxime, a carbamoyl group, a N-hydroxycarbamoyl group, a formylamino group, substituted or non-substituted C₁-C₈-alkyl, substituted or non-substituted tri(C₁-C₈-alkyl)silyl-C₁-C₈-alkyl, substituted or non-substituted C3-C8-cycloalkyl, substituted or non-substituted tri(C1-C8-alkyl)silyl-C3-C8-cycloalkyl, substituted or non-substituted C₁-C₈-halogenoalkyl having 1 to 5 halogen atoms, substituted or non-substituted C3-C8-halogenocycloalkyl having 1 to 5 halogen atoms, a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₂-C₈-alkynyloxy, substituted or non-substituted C₃-C₈-halogenoalkynyloxy having 1 to 5 halogen atoms, substituted or non-substituted C₁-C₈-alkylcarbonyl, substituted or non-substituted N—(C₁-C₈-alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbamothioyl, substituted or non-substituted di-C₁-C₈-alkylcarbamothioyl, substituted or non-substituted N—C₁-C₈-alkyloxycarbamothioyll, substituted or non-substituted C₁-C₈-alkoxycarbamothioyl, substituted or non-substituted N—C₁-C₈-alkyl-C₁-C₈-alkoxycarbamothioyl, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted C₁-C₈-alkylaminosulfamoyl, substituted or non-substituted di-C₁-C₈-alkylaminosulfamoyl, substituted or non-substituted aryl, substituted or non-substituted aryl-[C₁-C₈]-alkyl;
G¹ and G² are independently selected in the list consisting of oxygen, sulfur, NR⁵, N—OR⁶ and N—NR⁷R⁸;
Q represents a substituted phenyl group of formula (VI) 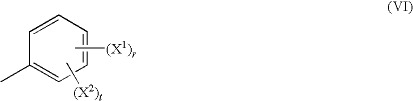
wherein:
X¹ independently represents a nitro group, a hydroxy group, a cyano group, an amino group, a sulphenyl group, a formyl group, a substituted or non-substituted carbaldehyde O—(C₁-C₈-alkyl)oxime, a formyloxy group, a formylamino group, a carbamoyl group, a N-hydroxycarbamoyl group, a pentafluoro-λ⁶-sulphenyl group, a formylamino group, substituted or non-substituted C₁-C₈-alkoxyamino group, substituted or non-substituted N—C₁-C₈-alkyl-(C₁-C₈-alkoxy)-amino group, substituted or non-substituted (C₁-C₈-alkylamino)-amino group, substituted or non-substituted N—C₁-C₈-alkyl-(C₁-C₈-alkylamino)-amino group, a substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C₃-C₈-cycloalkyl, substituted or non-substituted tri(C₁-C₈-alkyl)silyl-C₃-C₈-cycloalkyl, substituted or non-substituted C₁-C₈-halogenoalkyl having 1 to 5 halogen atoms, substituted or non-substituted C3-C8-halogenocycloalkyl having 1 to 5 halogen atoms, a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms, substituted or non-substituted C₁-C₈-alkylsulphenyl, substituted or non-substituted C₁-C₈-halogenoalkylsulphenyl having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms, substituted or non-substituted C₁-C₈-alkylcarbonyl, substituted or non-substituted N—(C₁-C₈-alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈-alkylcarbamoylamino, substituted or non-substituted C₁-C₈-halogenoalkylcarbamoylamino having 1 to 5 halogen atoms, substituted or non-substituted di-C₁-C₈-alkylcarbamoylamino, substituted or non-substituted di-C₁-C₈-halogenoalkylcarbamoylamino having 1 to 5 halogen atoms, substituted or non-substituted N—C₁-C₈-alkyl-(C₁-C₈-alkylcarbamoyl)amino, substituted or non-substituted N—C₁-C₈-alkyl-(C₁-C₈-halogenoalkylcarbamoyl)amino having 1 to 5 halogen atoms, substituted or non-substituted N—C₁-C₈-alkyl-(di-C₁-C₈-alkylcarbamoyl)amino, substituted or non-substituted N—C₁-C₈-alkyl-(di-C₁-C₈-halogenoalkylcarbamoyl)amino having 1 to 5 halogen atoms, substituted or non-substituted C₁-C₈-alkylaminocarbonyloxy, substituted or non-substituted di-C₁-C₈-alkylaminocarbonyloxy, substituted or non-substituted C₁-C₈-alkylcarbamothioyl, substituted or non-substituted di-C₁-C₈-alkylcarbamothioyl, substituted or non-substituted N—C₁-C₈-alkyloxycarbamothioyll, substituted or non-substituted C₁-C₈-alkoxycarbamothioyl, substituted or non-substituted N—C₁-C₈-alkyl-C₁-C₈-alkoxycarbamothioyll, substituted or non-substituted C₁-C₈-alkylthioylamino, substituted or non-substituted C₁-C₈-halogenoalkylthioylamino having 1 to 5 halogen atoms, substituted or non-substituted (C₁-C₈-alkyl-carbamothioyl)-oxy, substituted or non-substituted substituted or non-substituted (di-C₁-C₈-alkyl-carbamothioyl)-oxy, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted C₁-C₈-alkylaminosulfamoyl, substituted or non-substituted di-C₁-C₈-alkylaminosulfamoyl, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, substituted or non-substituted (benzyloxyimino)-C₁-C₆-alkyl, substituted or non-substituted benzyloxy, substituted or non-substituted benzylsulphenyl, substituted or non-substituted benzylamino, substituted or non-substituted phenoxy, substituted or non-substituted phenylsulphenyl, substituted or non-substituted phenylamino, substituted or non-substituted aryl, substituted or non-substituted aryl-[C₁-C₈]alkyl, substituted or non-substituted tri(C₁-C₈-alkyl)-silyloxy, substituted or non-substituted C₁-C₈-alkylsulfenylamino, substituted or non-substituted C₁-C₈-halogenoalkylsulphinylamino having 1 to 5 halogen atoms, substituted or non-substituted C₁-C₈-alkylsulphonylamino, substituted or non-substituted C₁-C₈-halogenoalkylsulphonylamino having I to 5 halogen atoms, substituted or non-substituted C₁-C₈-alkoxysulphonylamino, substituted or non-substituted C₁-C₈-halogenoxysulphonylamino having 1 to 5 halogen atoms, substituted or non-substituted tri(C₁-C₈-alkyl)-silyl, substituted or non-substituted (C₁-C₆-alkylideneamino)oxy, substituted or non-substituted (C₁-C₆-alkenylideneamino)oxy, substituted or non-substituted (C₁-C₆-alkynylideneamino)oxy, substituted or non-substituted (benzylideneamino)oxy; and
X² independently represents a halogen atom, a nitro group, a hydroxy group, a cyano group, an amino group, a sulphenyl group, a formyl group, a substituted or non-substituted carbaldehyde O—(C₁-C₈-alkyl)oxime, a formyloxy group, a formylamino group, a carbamoyl group, a N-hydroxycarbamoyl group, a pentafluoro-λ⁶-sulphenyl group, a formylamino group, substituted or non-substituted C₁-C₈-alkoxyamino group, substituted or non-substituted N—C₁-C₈-alkyl-(C₁-C₈-alkoxy)-amino group, substituted or non-substituted (C₁-C₈-alkylamino)-amino group, substituted or non-substituted N—C₁-C₈-alkyl-(C₁-C₈-alkylamino)-amino group, a substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C3-C8-cycloalkyl, substituted or non-substituted tri(C1-C8-alkyl)silyl-C3-C8-cycloalkyl, substituted or non-substituted C₁-C₈-halogenoalkyl having 1 to 5 halogen atoms, substituted or non-substituted C3-C8-halogenocycloalkyl having 1 to 5 halogen atoms, a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms, substituted or non-substituted C₁-C₈-alkylsulphenyl, substituted or non-substituted C₁-C₈-halogenoalkylsulphenyl having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈halogenoalkynyloxy having 1 to 5 halogen atoms, substituted or non-substituted C₁-C₈-alkylcarbonyl, substituted or non-substituted N—(C₁-C₈-alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈-alkylcarbamoylamino, substituted or non-substituted C₁-C₈-halogenoalkylcarbamoylamino having 1 to 5 halogen atoms, substituted or non-substituted di-C₁-C₈-alkylcarbamoylamino, substituted or non-substituted di-C₁-C₈-halogenoalkylcarbamoylamino having 1 to 5 halogen atoms, substituted or non-substituted N—C₁-C₈-alkyl-(C₁-C₈-alkylcarbamoyl)amino, substituted or non-substituted N—C₁-C₈-alkyl-(C₁-C₈-halogenoalkylcarbamoyl)amino having 1 to 5 halogen atoms, substituted or non-substituted N—C₁-C₈alkyl-(di-C₁-C₈-alkylcarbamoyl)amino, substituted or non-substituted N—C₁-C₈-alkyl-(di-C₁-C₈-halogenoalkylcarbamoyl)amino having 1 to 5 halogen atoms, substituted or non-substituted C₁-C₈-alkylaminocarbonyloxy, substituted or non-substituted di-C₁-C₈-alkylaminocarbonyloxy, substituted or non-substituted C₁-C₈-alkylcarbamothioyl, substituted or non-substituted di-C₁-C₈-alkylcarbamothioyl, substituted or non-substituted N—C₁-C₈-alkyloxycarbamothioyll, substituted or non-substituted C₁-C₈-alkoxycarbamothioyl, substituted or non-substituted N—C₁-C₈-alkyl-C₁-C₈-alkoxycarbamothioyll, substituted or non-substituted C₁-C₈-alkylthioylamino, substituted or non-substituted C₁-C₈-halogenoalkylthioylamino having 1 to 5 halogen atoms, substituted or non-substituted (C₁-C₈-alkyl-carbamothioyl)-oxy, substituted or non-substituted substituted or non-substituted (di-C₁-C₈-alkyl-carbamothioyl)-oxy, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted C₁-C₈-alkylaminosulfamoyl, substituted or non-substituted di-C₁-C₈-alkylaminosulfamoyl, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, substituted or non-substituted (benzyloxyimino)-C₁-C₆-alkyl, substituted or non-substituted benzyloxy, substituted or non-substituted benzylsulphenyl, substituted or non-substituted benzylamino, substituted or non-substituted phenoxy, substituted or non-substituted phenylsulphenyl, substituted or non-substituted phenylamino, substituted or non-substituted aryl, substituted or non-substituted aryl-[C₁-C₈]-alkyl, substituted or non-substituted tri(C₁-C₈-alkyl)-silyloxy, substituted or non-substituted C₁-C₈-alkylsulfenylamino, substituted or non-substituted C₁-C₈-halogenoalkylsulphinylamino having 1 to 5 halogen atoms, substituted or non-substituted C₁-C₈-alkylsulphonylamino, substituted or non-substituted C₁-C₈-halogenoalkylsulphonylamino having 1 to 5 halogen atoms, substituted or non-substituted C₁-C₈-alkoxysulphonylamino, substituted or non-substituted C₁-C₈-halogenoxysulphonylamino having 1 to 5 halogen atoms, substituted or non-substituted tri(C₁-C₈-alkyl)-silyl, substituted or non-substituted (C₁-C₆-alkylideneamino)oxy, substituted or non-substituted (C₁-C₆-alkenylideneamino)oxy, substituted or non-substituted (C₁-C₆-alkynylideneamino)oxy, substituted or non-substituted (benzylideneamino)oxy; and
r represents 1, 2, 3, 4 or 5;
t represents 0, 1, 2, 3, 4 or 5 and the sum of r and t equals to 2, 3, 4 or 5;
R¹, R², R³ and R⁴ independently represent a hydrogen atom, a halogen atom, a cyano group, substituted or non-substituted C₁-C₈-alkyl, substituted or non-substituted C3-C8-cycloalkyl, substituted or non-substituted C₁-C₈-halogenoalkyl having 1 to 5 halogen atoms, substituted or non-substituted C3-C8-halogenocycloalkyl having 1 to 5 halogen atoms, a C₂-C₈-alkenyl, substituted or non-substituted C₂-C₈-alkynyl, substituted or non-substituted C₁-C₈-alkoxy, substituted or non-substituted C₁-C₈-halogenoalkoxy having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms,
R⁵ to R⁸ independently represent a hydrogen atom, a halogen atom, a cyano group, substituted or non-substituted C₁-C₈-alkyl, substituted or non-substituted C3-C8-cycloalkyl, substituted or non-substituted C₁-C₈-halogenoalkyl having 1 to 5 halogen atoms, substituted or non-substituted C3-C8-halogenocycloalkyl having 1 to 5 halogen atoms, a C₂-C₈-alkenyl, substituted or non-substituted C₂-C₈-alkynyl, substituted or non-substituted C₁-C₈-alkoxy, substituted or non-substituted C₁-C₈-halogenoalkoxy having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms, substituted or non-substituted aryl, substituted or non-substituted aryl-[C₁-C₈]-alkyl; as well as salts, N-oxides, metallic complexes and metalloidic complexes thereof.

2. A compound according to claim 1 selected in the list of compounds of formula (Ia), (Ib), (Ic) and (Id) 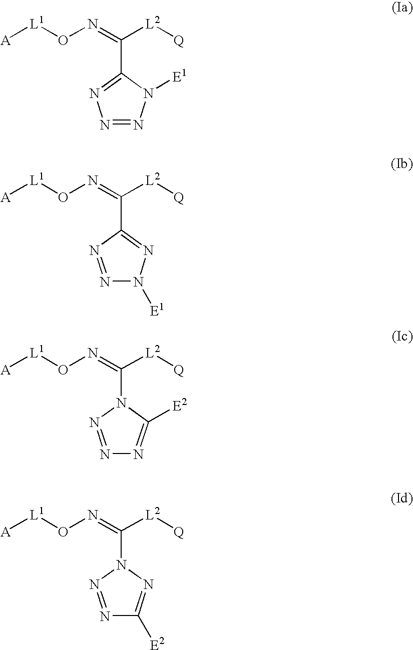
wherein
E¹ represents a hydrogen atom, a formyl group, a substituted or non-substituted carbaldehyde O—(C₁-C₈-alkyl)oxime, a carbamoyl group, a N-hydroxycarbamoyl group, a substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C3-C8-cycloalkyl, substituted or non-substituted tri(C1-C8-alkyl)silyl-C3-C8-cycloalkyl, substituted or non-substituted C₁-C₈-halogenoalkyl having 1 to 5 halogen atoms, substituted or non-substituted C3-C8-halogenocycloalkyl having 1 to 5 halogen atoms, a C₂-C₈-alkenyl, substituted or non-substituted C₂-C₈-alkynyl, substituted or non-substituted C₁-C₈-alkylcarbonyl, substituted or non-substituted N—(C₁-C₈-alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈alkoxycarbonyl, substituted or non-substituted C₁-C₈-halogenoalkoxycarbonyl having 1 to 5 halogen atoms, substituted or non-substituted C₁-C₈-alkylcarbamothioyl, substituted or non-substituted di-C₁-C₈-alkylcarbamothioyl, substituted or non-substituted N—C₁-C₈-alkyloxycarbamothioyl, substituted or non-substituted C₁-C₈-alkoxycarbamothioyl, substituted or non-substituted N—C₁-C₈-alkyl-C₁-C₈-alkoxycarbamothioyl, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted C₁-C₈-alkylaminosulfamoyl, substituted or non-substituted di-C₁-C₈-alkylaminosulfamoyl, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, substituted or non-substituted (benzyloxyimino)-C₁-C₆-alkyl, substituted or non-substituted aryl, substituted or non-substituted aryl-[C₁-C₈]-alkyl, substituted or non-substituted tri(C₁-C₈-alkyl)-silyl;
E² represents a hydrogen atom, a halogen atom, a nitro group, a hydroxy group, a cyano group, an amino group, a sulphenyl group, a formyl group, a substituted or non-substituted carbaldehyde O—(C₁-C₈-alkypoxime, a formyloxy group, a formylamino group, a carbamoyl group, a N-hydroxycarbamoyl group, a pentafluoro-λ⁶-sulphenyl group, a formylamino group, substituted or non-substituted C₁-C₈-alkoxyamino group, substituted or non-substituted N—C₁-C₈-alkyl-(C₁-C₈-alkoxy)-amino group, substituted or non-substituted (C₁-C₈-alkylamino)-amino group, substituted or non-substituted N—C₁-C₈-alkyl-(C₁-C₈-alkylamino)-amino group, a substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C3-C8-cycloalkyl, substituted or non-substituted tri(C1-C8-alkyl)silyl-C3-C8-cycloalkyl, substituted or non-substituted C₁-C₈-halogenoalkyl having 1 to 5 halogen atoms, substituted or non-substituted C3-C8-halogenocycloalkyl having 1 to 5 halogen atoms, a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms, substituted or non-substituted C₁-C₈-alkylsulphenyl, substituted or non-substituted C₁-C₈-halogenoalkylsulphenyl having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms, substituted or non-substituted C₁-C₈-alkylcarbonyl, substituted or non-substituted N—(C₁-C₈-alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈-alkylcarbamoylamino, substituted or non-substituted C₁-C₈-halogenoalkylcarbamoylamino having 1 to 5 halogen atoms, substituted or non-substituted di-C₁-C₈-alkylcarbamoylamino, substituted or non-substituted di-C₁-C₈-halogenoalkylcarbamoylamino having 1 to 5 halogen atoms, substituted or non-substituted N—C₁-C₈-alkyl-(C₁-C₈-alkylcarbamoyl)amino, substituted or non-substituted N—C₁-C₈-alkyl-(C₁-C₈-halogenoalkylcarbamoyl)amino having 1 to 5 halogen atoms, substituted or non-substituted N—C₁-C₈-alkyl-(di-C₁-C₈-alkylcarbamoyl)amino, substituted or non-substituted N—C₁-C₈-alkyl-(di-C₁-C₈-halogenoalkylcarbamoyl)amino having 1 to 5 halogen atoms, substituted or non-substituted C₁-C₈-alkylaminocarbonyloxy, substituted or non-substituted di-C₁-C₈-alkylaminocarbonyloxy, substituted or non-substituted C₁-C₈-alkylcarbamothioyl, substituted or non-substituted di-C₁-C₈-alkylcarbamothioyl, substituted or non-substituted N—C₁-C₈-alkyloxycarbamothioyll, substituted or non-substituted C₁-C₈-alkoxycarbamothioyl, substituted or non-substituted N—C₁-C₈-alkyl-C₁-C₈-alkoxycarbamothioyll, substituted or non-substituted C₁-C₈-alkylthioylamino, substituted or non-substituted C₁-C₈-halogenoalkylthioylamino having 1 to 5 halogen atoms, substituted or non-substituted (C₁-C₈-alkyl-carbamothioyl)-oxy, substituted or non-substituted substituted or non-substituted (di-C₁-C₈-alkyl-carbamothioyl)-oxy, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted C₁-C₈-alkylaminosulfamoyl, substituted or non-substituted di-C₁-C₈-alkylaminosulfamoyl, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, substituted or non-substituted (benzyloxyimino)-C₁-C₆-alkyl, substituted or non-substituted benzyloxy, substituted or non-substituted benzylsulphenyl, substituted or non-substituted benzylamino, substituted or non-substituted phenoxy, substituted or non-substituted phenylsulphenyl, substituted or non-substituted phenylamino, substituted or non-substituted aryl, substituted or non-substituted aryl-[C₁-C₈]-alkyl, substituted or non-substituted tri(C₁-C₈-alkyl)-silyloxy, substituted or non-substituted C₁-C₈-alkylsulfenylamino, substituted or non-substituted C₁-C₈-halogenoalkylsulphinylamino having 1 to 5 halogen atoms, substituted or non-substituted C₁-C₈-alkylsulphonylamino, substituted or non-substituted C₁-C₈-halogenoalkylsulphonylamino having 1 to 5 halogen atoms, substituted or non-substituted C₁-C₈-alkoxysulphonylamino, substituted or non-substituted C₁-C₈-halogenoxysulphonylamino having 1 to 5 halogen atoms, substituted or non-substituted tri(C₁-C₈-alkyl)-silyl, substituted or non-substituted (C₁-C₆-alkylideneamino)oxy, substituted or non-substituted (C₁-C₆-alkenylideneamino)oxy, substituted or non-substituted (C₁-C₆-alkynylideneamino)oxy, substituted or non-substituted (benzylideneamino)oxy.

3. A compound according to claim 1 wherein L¹ represents a direct bond or a divalent group selected in the list consisting of
—(CR¹R²)ₙ— —C(═O)—(CR¹R²)ₚ—
—(CR¹R²)ₘ—O— —(CR¹R²)ₘ—C(═O)—O—
—(CR¹R²)ₘ—NH— —(CR¹R²)ₘ—C(═O)—NH—
—(CR¹R²)ₘ—C(═O)— —(CR¹R²)ₘ—NH—C(═O)
wherein
n represents 1 or 2;
m and p independently represent 0 or 1;
R¹ and R² independently represent a hydrogen atom, a halogen atom, a cyano group, substituted or non-substituted C₁-C₈-alkyl, substituted or non-substituted C3-C8-cycloalkyl, substituted or non-substituted C₁-C₈-halogenoalkyl having 1 to 5 halogen atoms, substituted or non-substituted C3-C8-halogenocycloalkyl having 1 to 5 halogen atoms, a C₂-C₈-alkenyl, substituted or non-substituted C₂-C₈-alkynyl, substituted or non-substituted C₁-C₈-alkoxy, substituted or non-substituted C₁-C₈-halogenoalkoxy having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms.

4. A compound according to claim 3 wherein L¹ represents a direct bond or a divalent group selected in the list consisting of —(CR¹R²)—, —C(═O)—(CR¹R²)— and —C(═O)—; wherein R¹ and R² are independently selected in the list consisting of hydrogen, halogen, methyl, ethyl, isopropyl, trifluoromethyl, difluoromethyl, allyl, ethynyl, propargyl, cyclopropyl, methoxy, trifluoromethoxy and cyano.

5. A compound according to claim 1 wherein L² represents a direct bond or a divalent group selected in the list consisting of
—(CR³R⁴)_{q}— —(CR³R⁴)ₐ—C(═O)—
—(CR³═CR⁴)— —(CR³R⁴)ₐ—C(═O)—O—
C≡C— —(CR³R⁴)ₐ—O—C(═O)—
—(CR³R⁴)ₐ—O— —(CR³R⁴)ₐ—C(═O)—NH—
—(CR³R⁴)ₐ—NH— —(CR³R⁴)ₐ—NH—C(═O)—
wherein
q and a independently represent 1 or 2;
R³ and R⁴ independently represent a hydrogen atom, a halogen atom, a cyano group, substituted or non-substituted C₁-C₈-alkyl, substituted or non-substituted C3-C8-cycloalkyl, substituted or non-substituted C₁-C₈-halogenoalkyl having 1 to 5 halogen atoms, substituted or non-substituted C3-C8-halogenocycloalkyl having 1 to 5 halogen atoms, a C₂-C₈-alkenyl, substituted or non-substituted C₂-C₈-alkynyl, substituted or non-substituted C₁-C₈-alkoxy, substituted or non-substituted C₁-C₈-halogenoalkoxy having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms.

6. A compound according to claim 5 wherein L² represents a direct bond or —(CR³R⁴)— wherein R³ and R⁴ are independently selected in the list consisting of hydrogen, halogen, methyl, ethyl, isopropyl, trifluoromethyl, difluoromethyl, allyl, ethynyl, propargyl, cyclopropyl, methoxy, trifluoromethoxy and cyano.

7. A compound according to claim 1 wherein A is selected in the list consisting of A¹ to A⁵⁸.

8. A compound according to claim 7 wherein A is selected in the list consisting of A², A⁶, A⁸, A¹¹, A¹², A¹³, A¹⁴, A¹⁵, A¹⁶, A¹⁷ and A¹⁸.

9. A compound according to claim 1 wherein Z¹ represents a hydrogen atom, a halogen atom, a hydroxy group, a cyano group, an amino group, a formyloxy group, a formylamino group, a carbamoyl group, a N-hydroxycarbamoyl group, a pentafluoro-λ⁶-sulphenyl group, a formylamino group, substituted or non-substituted C₁-C₈-alkoxyamino group, substituted or non-substituted N—C₁-C₈-alkyl-(C₁-C₈-alkoxy)-amino group, a substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C₃-C₈-cycloalkyl, substituted or non-substituted tri(C₁-C₉-alkyl)silyl-C₃-C₈-cycloalkyl, substituted or non-substituted C₁-C₈-halogenoalkyl having 1 to 5 halogen atoms, substituted or non-substituted C₃-C₈-halogenocycloalkyl having 1 to 5 halogen atoms, a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted (C₁-C₈-alkoxycarbonyl)amino, substituted or non-substituted (C₂-C₈-alkenyloxycarbonyl)amino, substituted or non-substituted (C₃-C₈-alkynyloxycarbonyl)amino, substituted or non-substituted (C₃-C₈-cycloalkoxycarbonyl)amino, substituted or non-substituted C₁-C₈-halogenoalkoxy having 1 to 5 halogen atoms, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkylcarbonyl, substituted or non-substituted N—(C₁-C₈-alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈-alkylcarbamoylamino, substituted or non-substituted C₁-C₈-halogenoalkylcarbamoylamino having 1 to 5 halogen atoms, substituted or non-substituted di-C₁-C₈-alkylcarbamoylamino, substituted or non-substituted di-C₁-C₈-halogenoalkylcarbamoylamino having 1 to 5 halogen atoms, substituted or non-substituted N—C₁-C₈-alkyl-(C₁-C₈-alkylcarbamoyl)amino, substituted or non-substituted N—C₁-C₈-alkyl-(C₁-C₈-halogenoalkylcarbamoyl)amino having 1 to 5 halogen atoms, substituted or non-substituted N—C₁-C₈-alkyl-(di-C₁-C₈-alkylcarbamoyl)amino, substituted or non-substituted N—C₁-C₈-alkyl-(di-C₁-C₈-halogenoalkylcarbamoyl)amino having 1 to 5 halogen atoms, substituted or non-substituted C₁-C₈-alkylaminocarbonyloxy, substituted or non-substituted di-C₁-C₈-alkylaminocarbonyloxy, substituted or non-substituted C₁-C₈-alkylcarbamothioyl, substituted or non-substituted di-C₁-C₈-alkylcarbamothioyl, substituted or non-substituted N—C₁-C₈-alkyloxycarbamothioyl, substituted or non-substituted C₁-C₈-alkoxycarbamothioyl, substituted or non-substituted N—C₁-C₈-alkyl-C₁-C₈-alkoxycarbamothioyl, substituted or non-substituted C₁-C₈-alkylthioylamino, substituted or non-substituted C₁-C₈-halogenoalkylthioylamino having 1 to 5 halogen atoms, substituted or non-substituted (C₁-C₈-alkyl-carbamothioyl)-oxy, substituted or non-substituted substituted or non-substituted (di-C₁-C₈-alkyl-carbamothioyl)-oxy, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkylaminosulfamoyl, substituted or non-substituted di-C₁-C₈-alkylaminosulfamoyl, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, substituted or non-substituted (benzyloxyimino)-C₁-C₆-alkyl, substituted or non-substituted benzyloxy, substituted or non-substituted benzylsulphenyl, substituted or non-substituted benzylamino, substituted or non-substituted phenoxy, substituted or non-substituted phenylsulphenyl, substituted or non-substituted phenylamino, substituted or non-substituted aryl, substituted or non-substituted aryl-[C₁-C₈]-alkyl, substituted or non-substituted tri(C₁-C₈-alkyl)-silyloxy, substituted or non-substituted C₁-C₈-halogenoalkylsulphinylamino having 1 to 5 halogen atoms, substituted or non-substituted C₁-C₈-alkylsulphonylamino, substituted or non-substituted C₁-C₈-halogenoalkylsulphonylamino having 1 to 5 halogen atoms, substituted or non-substituted C₁-C₈-alkoxysulphonylamino, substituted or non-substituted C₁-C₈-halogenoxysulphonylamino having 1 to 5 halogen atoms, substituted or non-substituted tri(C₁-C₈-alkyl)-silyl, substituted or non-substituted (C₁-C₆-alkylideneamino)oxy, substituted or non-substituted (C₁-C₆-alkenylideneamino)oxy, substituted or non-substituted (C₁-C₆-alkynylideneamino)oxy, substituted or non-substituted (benzylideneamino)oxy.

10. A compound according to claim 9 wherein Z¹ represents a hydrogen atom, a halogen atom, a cyano group, an amino group, a formyloxy group, a formylamino group, a carbamoyl group, a N-hydroxycarbamoyl group, a formylamino group, substituted or non-substituted C₁-C₈-alkoxyamino group, substituted or non-substituted N—C₁-C₈-alkyl-(C₁-C₈-alkoxy)-amino group, substituted or non-substituted C₁-C₈-alkyl, substituted or non-substituted tri(C₁-C₈-alkyl)silyl-C₁-C₈-alkyl, substituted or non-substituted C₃-C₈-cycloalkyl, substituted or non-substituted C₁-C₈-halogenoalkyl having 1 to 5 halogen atoms, a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted (C₁-C₈-alkoxycarbonyl)amino, substituted or non-substituted (C₂-C₈-alkenyloxycarbonyl)amino, substituted or non-substituted (C₃-C₈-alkynyloxycarbonyl)amino, substituted or non-substituted (C₃-C₈-cycloalkoxycarbonyl)amino, substituted or non-substituted C₁-C₈-halogenoalkoxy having 1 to 5 halogen atoms, substituted or non-substituted C₁-C₈-alkylsulphenyl, substituted or non-substituted C₁-C₈-alkylcarbonyl, substituted or non-substituted N—(C₁-C₈-alkoxy)-C₁-C₈-alkanimidoyl, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈-alkylcarbamoylamino, substituted or non-substituted C₁-C₈-halogenoalkylcarbamoylamino having 1 to 5 halogen atoms, substituted or non-substituted di-C₁-C₈-alkylcarbamoylamino, substituted or non-substituted di-C₁-C₈-halogenoalkylcarbamoylamino having 1 to 5 halogen atoms, substituted or non-substituted N—C₁-C₈-alkyl-(C₁-C₈-alkylcarbamoylamino, substituted or non-substituted N—C₁-C₈-alkyl-(C₁-C₈-halogenoalkylcarbamoyl)amino having 1 to 5 halogen atoms, substituted or non-substituted N—C₁-C₈-alkyl-(di-C₁-C₈-alkylcarbamoyl)amino, substituted or non-substituted N—C₁-C₈-alkyl-(di-C₁-C₈-halogenoalkylcarbamoyl)amino having 1 to 5 halogen atoms, substituted or non-substituted C₁-C₈-alkylcarbamothioyl, substituted or non-substituted di-C₁-C₈-alkylcarbamothioyl, substituted or non-substituted N—C₁-C₈-alkyloxycarbamothioyl, substituted or non-substituted C₁-C₈-alkoxycarbamothioyl, substituted or non-substituted N—C₁-C₈-alkyl-C₁-C₈-alkoxycarbamothioyl, substituted or non-substituted C₁-C₈-alkylthioylamino, substituted or non-substituted C₁-C₈-halogenoalkylthioylamino having 1 to 5 halogen atoms, substituted or non-substituted benzylamino, substituted or non-substituted phenoxy, substituted or non-substituted phenylamino, substituted or non-substituted aryl, substituted or non-substituted aryl-[C₁-C₈]-alkyl, substituted or non-substituted C₁-C₈-halogenoalkylsulphinylamino having 1 to 5 halogen atoms, substituted or non-substituted C₁-C₈-alkylsulphonylamino, substituted or non-substituted C₁-C₈-halogenoalkylsulphonylamino having 1 to 5 halogen atoms.

11. A compound according to claim 1 wherein Z², Z³, Z⁴, Z⁵, Z⁶, Z⁷, Z⁸ and Z⁹ independently represent a hydrogen atom, a halogen atom, a cyano group, substituted or non-substituted C₁-C₈-alkyl, substituted or non-substituted C₃-C₈-cycloalkyl, substituted or non-substituted C₁-C₈-halogenoalkyl having 1 to 5 halogen atoms, a C₂-C₈-alkenyl, substituted or non-substituted C₂-C₈-alkynyl, substituted or non-substituted C₁-C₈-alkoxy, substituted or non-substituted C₁-C₈-halogenoalkoxy having 1 to 5 halogen atoms, substituted or non-substituted phenoxy, substituted or non-substituted aryl, substituted or non-substituted aryl-[C₁-C₈]-alkyl.

12. A compound according to claim 11 wherein Z², Z³, Z⁴, Z⁵, Z⁶, Z⁷, Z⁸ and Z⁹ are independently selected in the list consisting of hydrogen, halogen, methyl, ethyl, isopropyl, isobutyl, terbutyl, trifluoromethyl, difluoromethyl, allyl, ethynyl, propargyl, cyclopropyl, methoxy, trifluoromethoxy, acetyl, and cyano.

13. A compound according to claim 1 wherein K¹ and K² are independently selected in the list consisting of hydrogen, methyl, ethyl, isopropyl, isobutyl, terbutyl, allyl, propargyl, cyclopropyl, acetyl, trifluoroacetyl and mesyl.

14. A compound according to claim 1 wherein X¹ independently represents a cyano group, an amino group, a sulphenyl group, a pentafluoro-λ⁶-sulphenyl group, substituted or non-substituted C₁-C₈-alkyl, substituted or non-substituted tri(C₁-C₈-alkyl)silyl-C₁-C₈-alkyl, substituted or non-substituted C3-C8-cycloalkyl, substituted or non-substituted tri(C1-C8-alkyl)silyl-C3-C8-cycloalkyl, substituted or non-substituted C₁-C₈-halogenoalkyl having 1 to 5 halogen atoms, substituted or non-substituted C3-C8-halogenocycloalkyl having 1 to 5 halogen atoms, a C₂-C₈-alkenyl, substituted or non-substituted C₂-C₈-alkynyl, substituted or non-substituted C₁-C₈-alkoxy, substituted or non-substituted C₁-C₈-halogenoalkoxy having 1 to 5 halogen atoms, substituted or non-substituted C₁-C₈-alkylsulphenyl, substituted or non-substituted C₁-C₈-halogenoalkylsulphenyl having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms, substituted or non-substituted C₁-C₈-alkylcarbonyl, substituted or non-substituted N—(C₁-C₈-alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbamothioyl, substituted or non-substituted di-C₁-C₈-alkylcarbamothioyl, substituted or non-substituted N—C₁-C₈-alkyloxycarbamothioyl, substituted or non-substituted C₁-C₈-alkoxycarbamothioyl, substituted or non-substituted N—C₁-C₈-alkyl-C₁-C₈-alkoxycarbamothioyl, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkyl-sulphonyl, substituted or non-substituted C₁-C₈-halogenoalkylsulphonyl having 1 to 5 halogen atoms, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, substituted or non-substituted (benzyloxyimino)-C₁-C₆-alkyl, substituted or non-substituted benzyloxy, substituted or non-substituted benzylsulphenyl, substituted or non-substituted phenoxy, substituted or non-substituted phenylsulphenyl, substituted or non-substituted aryl, substituted or non-substituted aryl-[C₁-C₈]-alkyl, substituted or non-substituted tri(C₁-C₈-alkyl)-silyloxy, substituted or non-substituted tri(C₁-C₈-alkyl)-silyl;

15. A compound according to claim 14 wherein X¹ independently represents a cyano group, an amino group, a sulphenyl group, a pentafluoro-λ⁶-sulphenyl group, substituted or non-substituted C₁-C₈-alkyl, substituted or non-substituted tri(C₁-C₈-alkyl)silyl-C₁-C₈-alkyl, substitute or non-substituted C3-C8-cycloalkyl, substituted or non-substituted tri(C1-C8-alkyl)silyl-C3-C8-cycloalkyl, substituted or non-substituted C₁-C₈-halogenoalkyl having 1 to 5 halogen atoms, substituted or non-substituted C3-C8-halogenocycloalkyl having 1 to 5 halogen atoms, a C₂-C₈-alkenyl, substituted or non-substituted C₂-C₈-alkynyl, substituted or non-substituted C₁-C₈-alkoxy, substituted or non-substituted C₁-C₈-halogenoalkoxy having 1 to 5 halogen atoms, substituted or non-substituted C₁-C₈-alkylsulphenyl, substituted or non-substituted C₁-C₈-halogenoalkylsulphenyl having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms, substituted or non-substituted C₁-C₈-alkylcarbonyl, substituted or non-substituted N—(C₁-C₈-alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbamothioyl, substituted or non-substituted di-C₁-C₈-alkylcarbamothioyl, substituted or non-substituted N—C₁-C₈-alkyloxycarbamothioyll, substituted or non-substituted C₁-C₈-alkoxycarbamothioyl, substituted or non-substituted N—C₁-C₈-alkyl-C₁-C₈-alkoxycarbamothioyl, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, substituted or non-substituted (benzyloxyimino)-C₁-C₆-alkyl, substituted or non-substituted benzyloxy, substituted or non-substituted benzylsulphenyl, substituted or non-substituted phenoxy, substituted or non-substituted phenylsulphenyl, substituted or non-substituted aryl, substituted or non-substituted aryl-[C₁-C₈]-alkyl, substituted or non-substituted tri(C₁-C₈-alkyl)-silyloxy, substituted or non-substituted tri(C₁-C₈-alkyl)-silyl.

16. A compound according to claim 15 wherein X¹ independently represents methyl, isopropyl, isobutyl, tertbutyl, trifluoromethyl, difluoromethyl, allyl, ethynyl, propargyl, cyclopropyl, benzyl, phenethyl, methoxy, trifluoromethoxy, acetyl, trifluoroacetyl and cyano.

17. A compound according to claim 1 wherein X² independently represents a halogen atom, a cyano group, an amino group, a sulphenyl group, a pentafluoro-λ⁶-sulphenyl group, substituted or non-substituted C₁-C₈-alkyl, substituted or non-substituted tri(C₁-C₈-alkyl)silyl-C₁-C₈-alkyl, substituted or non-substituted C3-C8-cycloalkyl, substituted or non-substituted tri(C1-C8-alkyl)silyl-C3-C8-cycloalkyl, substituted or non-substituted C₁-C₈-halogenoalkyl having 1 to 5 halogen atoms, substituted or non-substituted C3-C8-halogenocycloalkyl having 1 to 5 halogen atoms, a C₂-C₈-alkenyl, substituted or non-substituted C₂-C₈-alkynyl, substituted or non-substituted C₁-C₈-alkoxy, substituted or non-substituted C₁-C₈-halogenoalkoxy having 1 to 5 halogen atoms, substituted or non-substituted C₁-C₈-alkylsulphenyl, substituted or non-substituted C₁-C₈-halogenoalkylsulphenyl having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms, substituted or non-substituted C₁-C₈-alkylcarbonyl, substituted or non-substituted N—(C₁-C₈-alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbamothioyl, substituted or non-substituted di-C₁-C₈-alkylcarbamothioyl, substituted or non-substituted N—C₁-C₈-alkyloxycarbamothioyll, substituted or non-substituted C₁-C₈-alkoxycarbamothioyl, substituted or non-substituted N—C₁-C₈-alkyl-C₁-C₈-alkoxycarbamothioyl, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkyl-sulphonyl, substituted or non-substituted C₁-C₈-halogenoalkylsulphonyl having 1 to 5 halogen atoms, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, substituted or non-substituted (benzyloxyimino)-C₁-C₆-alkyl, substituted or non-substituted benzyloxy, substituted or non-substituted benzylsulphenyl, substituted or non-substituted phenoxy, substituted or non-substituted phenylsulphenyl, substituted or non-substituted aryl, substituted or non-substituted aryl-[C₁-C₈]-alkyl, substituted or non-substituted tri(C₁-C₈-alkyl)-silyloxy, substituted or non-substituted tri(C₁-C₈-alkyl)-silyl.

18. A compound according to claim 17 wherein X² independently represents a halogen atom, methyl, isopropyl, isobutyl, tertbutyl, trifluoromethyl, difluoromethyl, allyl, ethynyl, propargyl, cyclopropyl, benzyl, phenethyl, methoxy, trifluoromethoxy, acetyl, trifluoroacetyl and cyano.

19. A compound according to claims 2 wherein E² represents a formyl group, methyl, ethyl, iso-propyl, allyl, propargyl, cyclopropyl, substituted or non-substituted C₁-C₈-alkylcarbonyl.

20. A compound according to claim 2 wherein E² represents a hydrogen atom, a halogen atom, a formyl group, a substituted or non-substituted carbaldehyde O—(C₁-C₈-alkyl)oxime, substituted or non-substituted C₁-C₈-alkyl, substituted or non-substituted C₃-C₈-cycloalkyl, substituted or non-substituted C₁-C₈-halogenoalkyl having 1 to 5 halogen atoms, a C₂-C₈-alkenyl, substituted or non-substituted C₂-C₈-alkynyl, substituted or non-substituted C₁-C₈-alkoxy, substituted or non-substituted C₁-C₈-halogenoalkoxy having 1 to 5 halogen atoms, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈alkylcarbonyloxy, substituted or non-substituted C₁-C₈-halogenoalkylcarbonyloxy having 1 to 5 halogen atoms, substituted or non-substituted C₁-C₈-alkylsulphenyl, substituted or non-substituted benzyloxy, substituted or non-substituted benzylsulphenyl, substituted or non-substituted phenoxy, substituted or non-substituted phenylsulphenyl, substituted or non-substituted aryl, substituted or non-substituted aryl-[C₁-C₈]-alkyl, substituted or non-substituted tri(C₁-C₈-alkyl)-silyl, substituted or non-substituted (C₁-C₆-alkylideneamino)oxy, substituted or non-substituted (C₁-C₆-alkenylideneamino)oxy, substituted or non-substituted (C₁-C₆-alkynylideneamino)oxy, substituted or non-substituted (benzylideneamino)oxy.

21. A compound according to claim 20 wherein E² represents a halogen atom, methyl, ethyl, isopropyl, trifluoromethyl, difluoromethyl, allyl, ethynyl, propargyl, cyclopropyl, cyano.

22. A compound of formula (II) 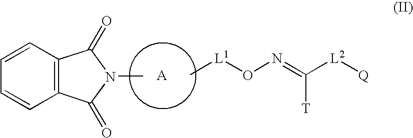
wherein
A is selected in the list consisting of A¹ to A¹¹⁶ 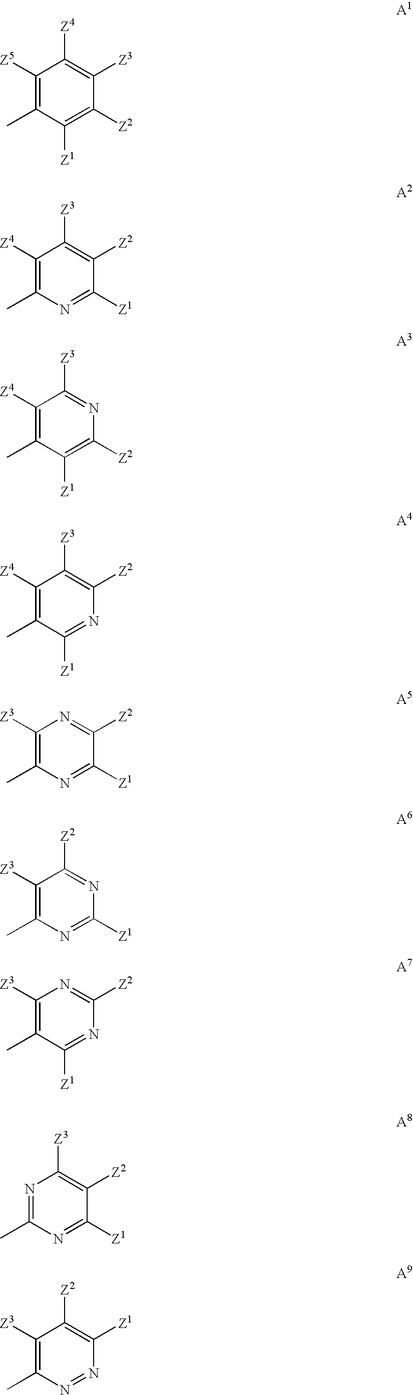 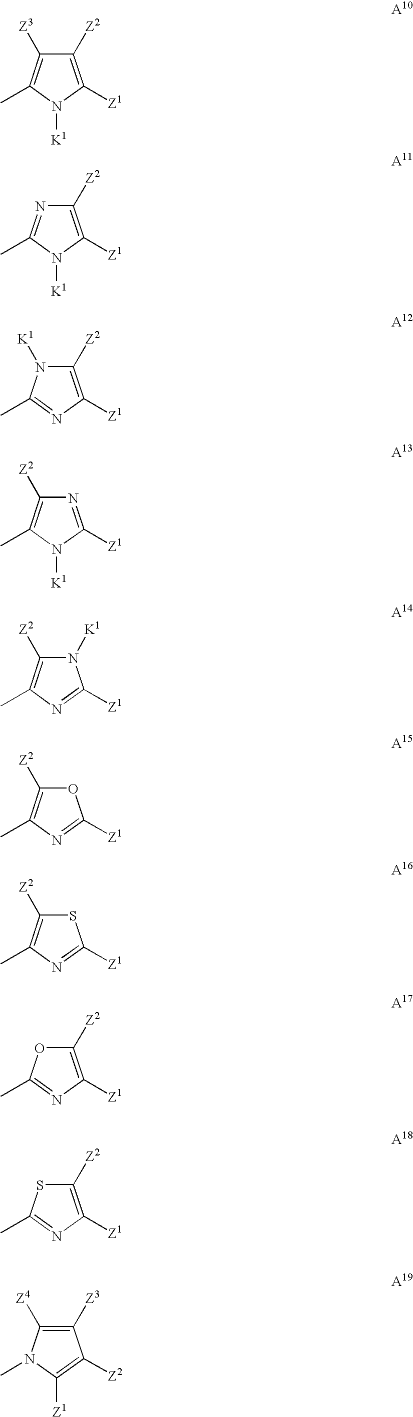 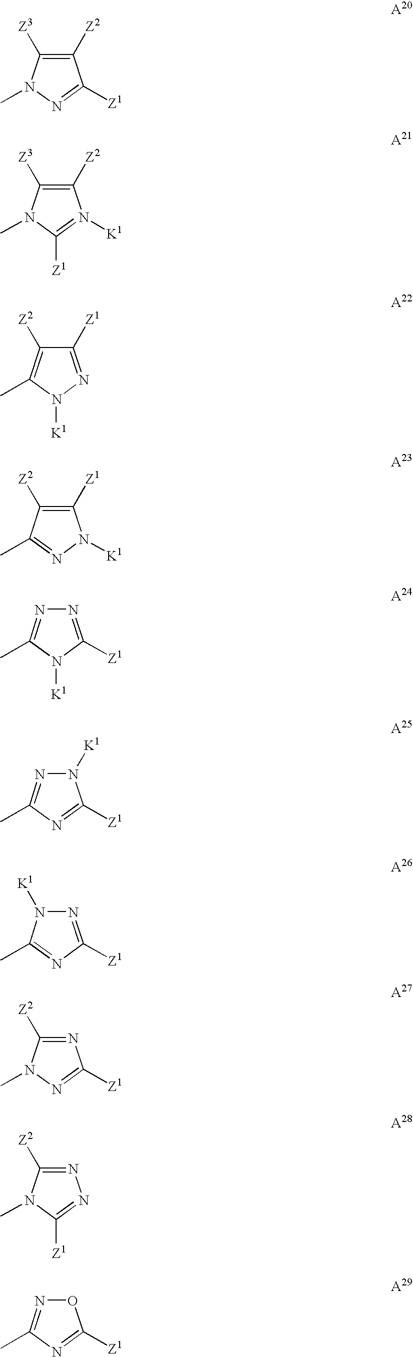 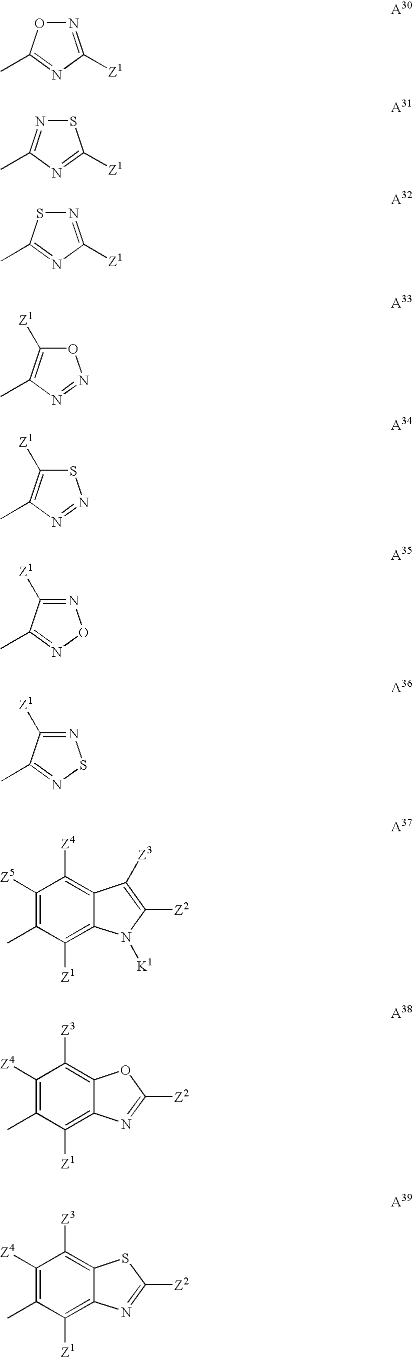 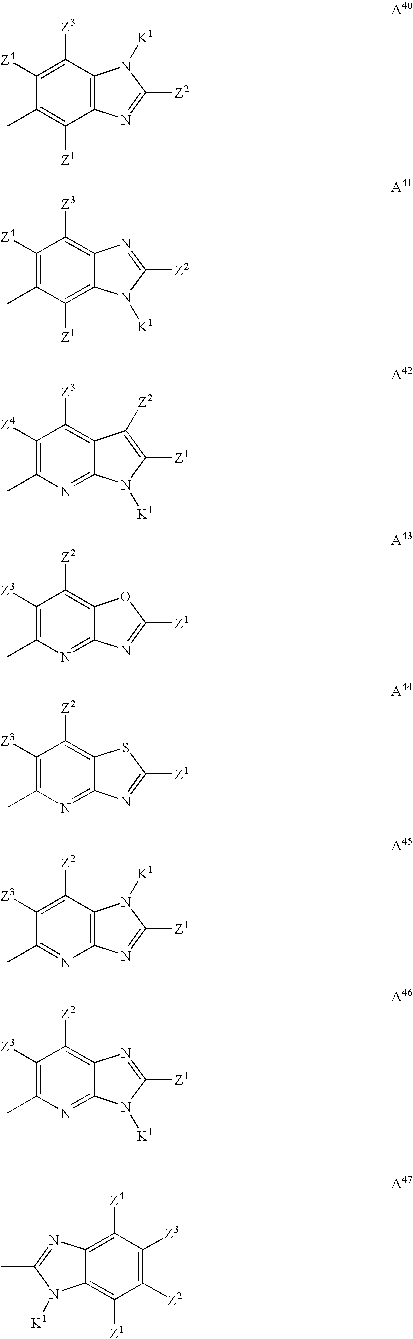 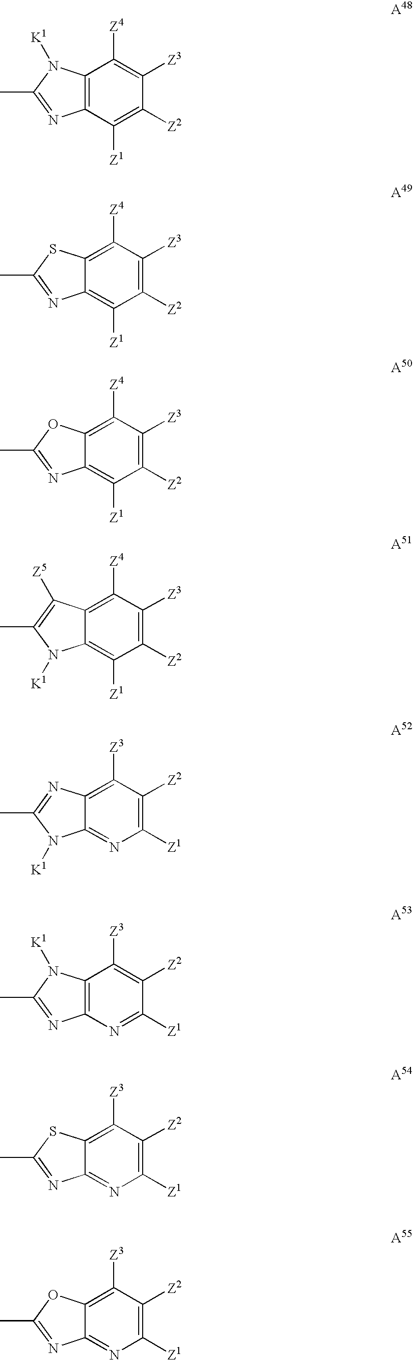 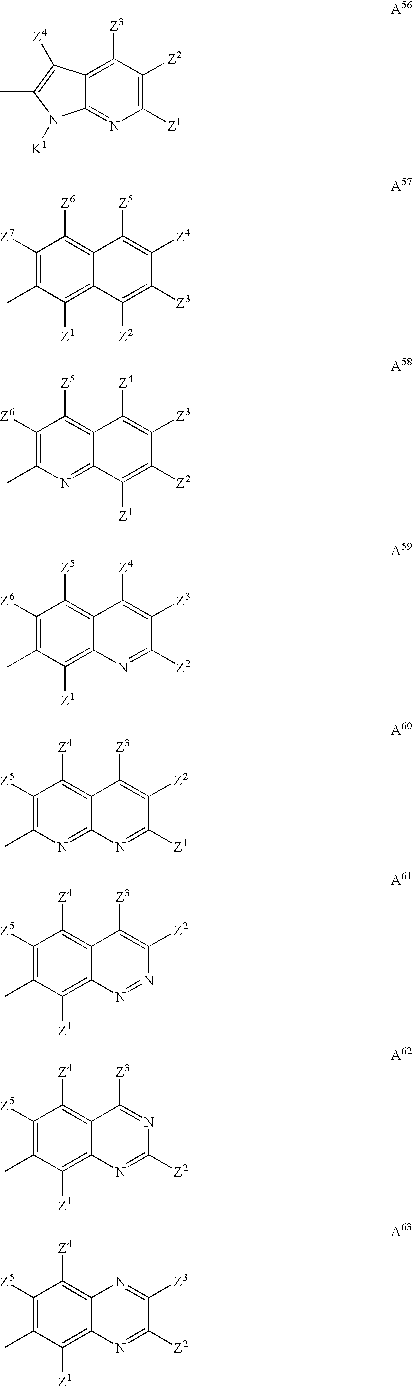 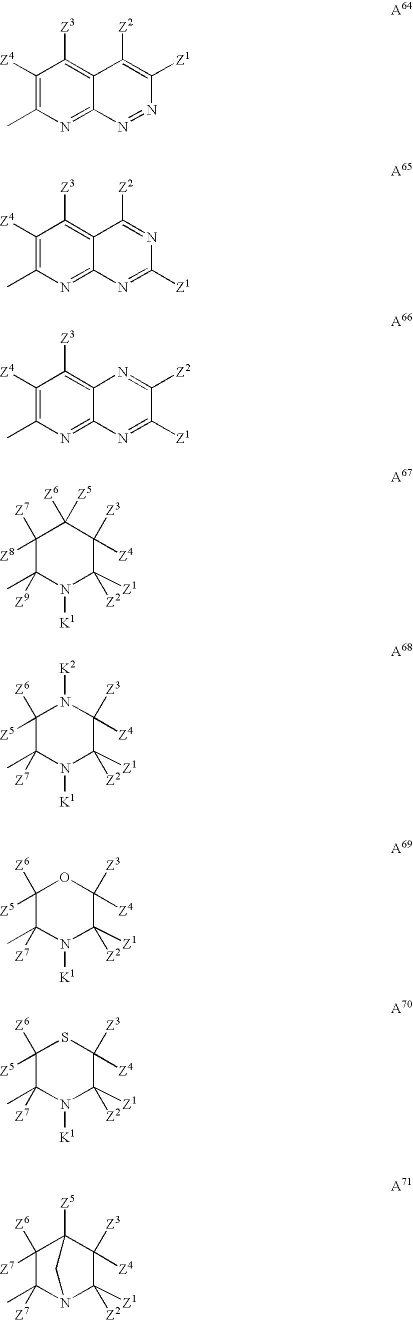 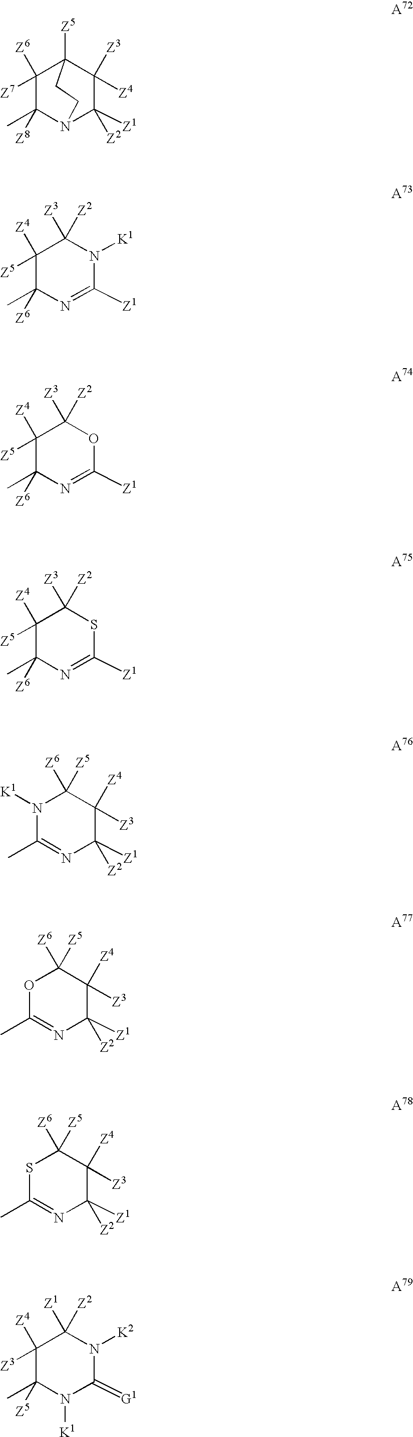 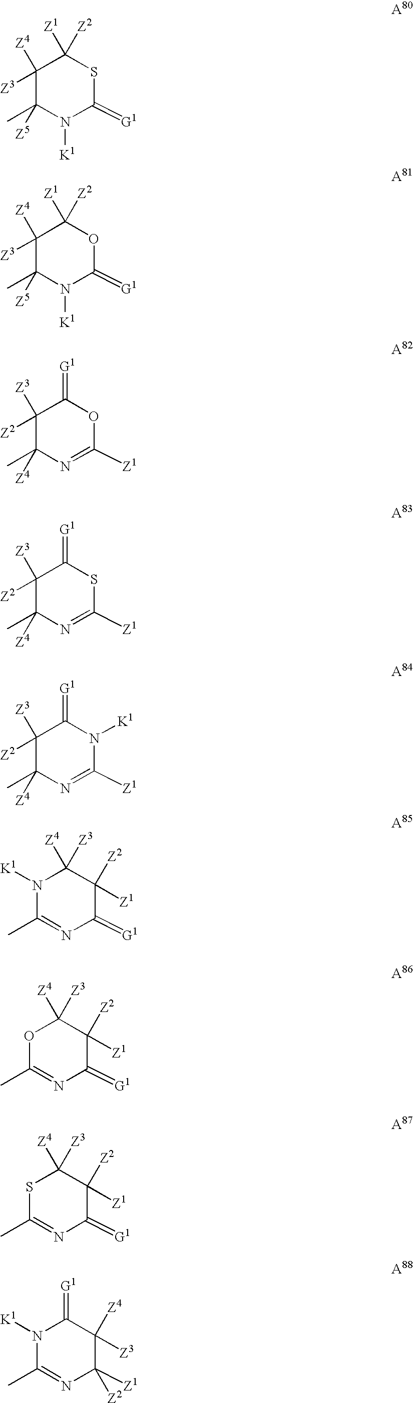 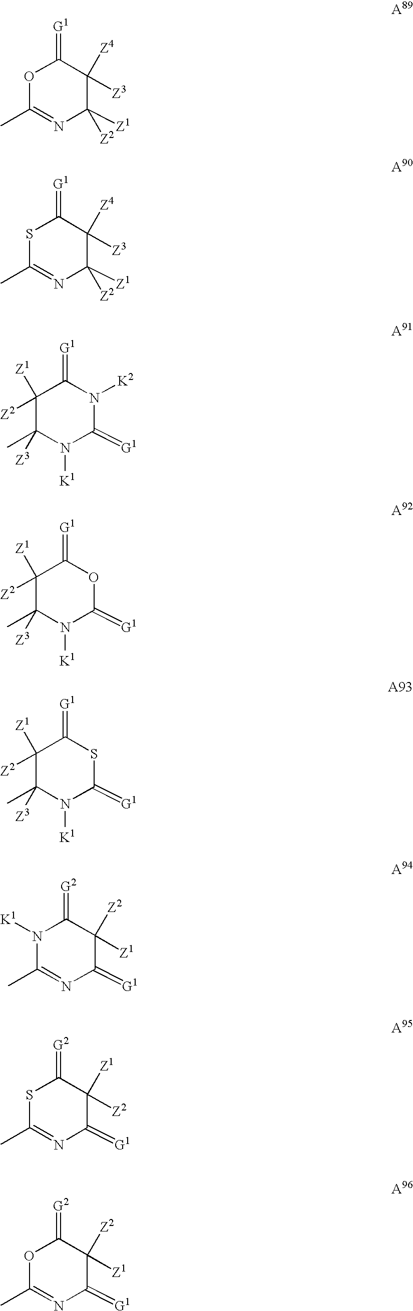 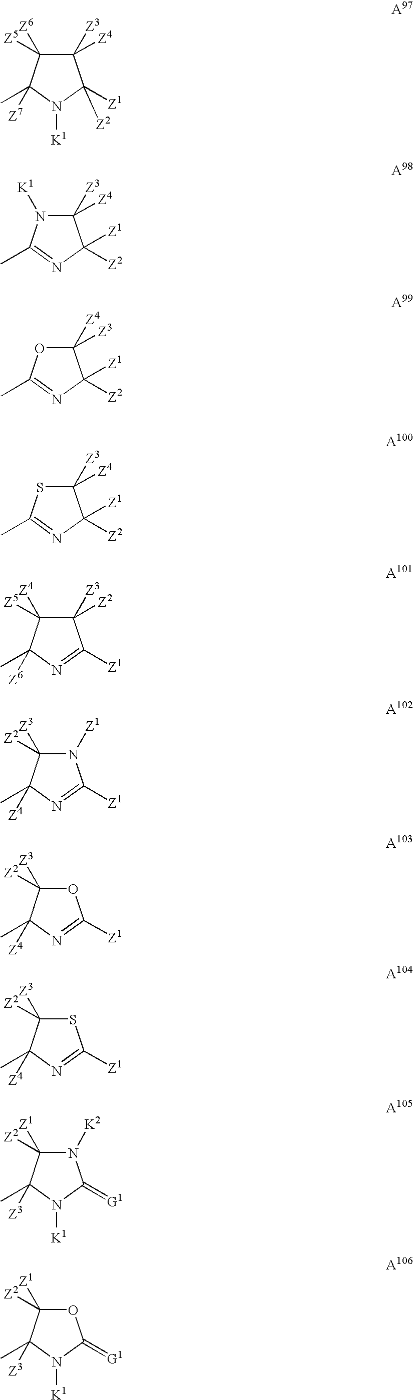 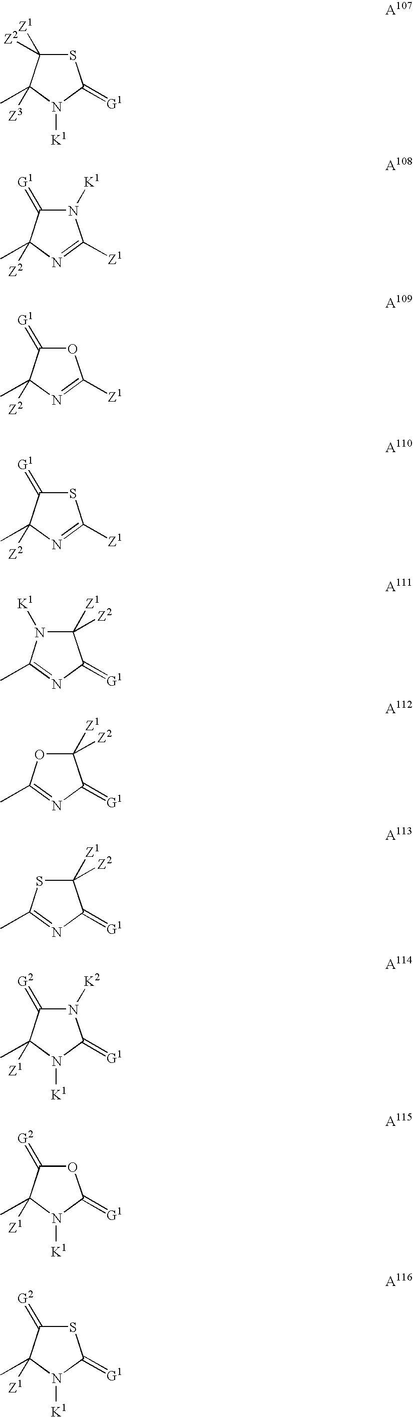
wherein
Z¹, Z², Z³, Z⁴, Z⁵, Z⁶, Z⁷, Z⁸ and Z⁹ independently represent a hydrogen atom, a halogen atom, a nitro group, a hydroxy group, a cyano group, an amino group, a sulphenyl group, a formyl group, a substituted or non-substituted carbaldehyde O—(C₁-C₈-alkyl)oxime, a formyloxy group, a formylamino group, a carbamoyl group, a N-hydroxycarbamoyl group, a pentafluoro-□⁶-sulphenyl group, a formylamino group, substituted or non-substituted C₁-C₈-alkoxyamino group, substituted or non-substituted N—C₁-C₈-alkyl-(C₁-C₈-alkoxy)-amino group, substituted or non-substituted (C₁-C₈-alkylamino)-amino group, substituted or non-substituted N—C₁-C₈-alkyl-(C₁-C₈-alkylamino)-amino group, a substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C₃-C₈-cycloalkyl, substituted or non-substituted tri(C₁-C₈-alkyl)silyl-C₃-C₈-cycloalkyl, substituted or non-substituted C₁-C₈-halogenoalkyl having 1 to 5 halogen atoms, substituted or non-substituted C₃-C₈-halogenocycloalkyl having 1 to 5 halogen atoms, a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted (C₁-C₈-alkoxycarbonyl)amino, substituted or non-substituted (C₃-C₈-cycloalkoxycarbonyl)amino, substituted or non-substituted C₁-C₈-halogenoalkoxy having 1 to 5 halogen atoms, substituted or non-substituted C₁-C₈-alkylsulphenyl, substituted or non-substituted C₁-C₈-halogenoalkylsulphenyl having 1 to 5 halogen atoms, substituted or non-substituted C₂-C₈-alkenyloxy, substituted or non-substituted (C₂-C₈-alkenyloxycarbonyl)amino, substituted or non-substituted C₂-C₈-halogenoalkenyloxy having 1 to 5 halogen atoms, substituted or non-substituted C₃-C₈-alkynyloxy, substituted or non-substituted (C₃-C₈-alkynyloxycarbonyl)amino, substituted or non-substituted C₃-C₈-halogenoalkynyloxy having 1 to 5 halogen atoms, substituted or non-substituted C₁-C₈-alkylcarbonyl, substituted or non-substituted N—(C₁-C₈-alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈-alkylcarbamoylamino, substituted or non-substituted C₁-C₈-halogenoalkylcarbamoylamino having 1 to 5 halogen atoms, substituted or non-substituted di-C₁-C₈-alkylcarbamoylamino, substituted or non-substituted di-C₁-C₈-halogenoalkylcarbamoylamino having 1 to 5 halogen atoms, substituted or non-substituted N—C₁-C₈-alkyl-(C₁-C₈-alkylcarbamoyl)amino, substituted or non-substituted N—C₁-C₈-alkyl-(C₁-C₈-halogenoalkylcarbamoyl)amino having 1 to 5 halogen atoms, substituted or non-substituted N—C₁-C₈-alkyl-(di-C₁-C₈-alkylcarbamoyl)amino, substituted or non-substituted N—C₁-C₈-alkyl-(di-C₁-C₈-halogenoalkylcarbamoyl)amino having 1 to 5 halogen atoms, substituted or non-substituted C₁-C₈-alkylaminocarbonyloxy, substituted or non-substituted di-C₁-C₈-alkylaminocarbonyloxy, substituted or non-substituted C₁-C₈-alkylcarbamothioyl, substituted or non-substituted di-C₁-C₈-alkylcarbamothioyl, substituted or non-substituted N—C₁-C₈-alkyloxycarbamothioyll, substituted or non-substituted C₁-C₈-alkoxycarbamothioyl, substituted or non-substituted N—C₁-C₈-alkyl -C₁-C₈-alkoxycarbamothioyll, substituted or non-substituted C₁-C₈-alkylthioylamino, substituted or non-substituted (C₁-C₈-alkoxythiocarbonyl)amino, substituted or non-substituted C₁-C₈-halogenoalkylthioylamino having 1 to 5 halogen atoms, substituted or non-substituted (C₁-C₈-alkyl-carbamothioyl)-oxy, substituted or non-substituted substituted or non-substituted (di-C₁-C₈-alkyl-carbamothioyl)-oxy, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted C₁-C₈-alkylaminosulfamoyl, substituted or non-substituted di-C₁-C₈alkylaminosulfamoyl, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, substituted or non-substituted (benzyloxyimino)-C₁-C₆-alkyl, substituted or non-substituted benzyloxy, substituted or non-substituted benzylsulphenyl, substituted or non-substituted benzylamino, substituted or non-substituted phenoxy, substituted or non-substituted phenylsulphenyl, substituted or non-substituted phenylamino, substituted or non-substituted aryl, substituted or non-substituted (arylcarbonyl)amino, substituted or non-substituted (heterocyclylcarbonyl)amino substituted or non-substituted aryl-[C₁-C₈]-alkyl, substituted or non-substituted tri(C₁-C₈-alkyl)-silyloxy, substituted or non-substituted C₁-C₈-alkylsulfenylamino, substituted or non-substituted C₁-C₈-halogenoalkylsulphinylamino having 1 to 5 halogen atoms, substituted or non-substituted C₁-C₈-alkylsulphonylamino, substituted or non-substituted C₁-C₈-halogenoalkylsulphonylamino having 1 to 5 halogen atoms, substituted or non-substituted C₁-C₈-alkoxysulphonylamino, substituted or non-substituted C₁-C₈-halogenoxysulphonylamino having 1 to 5 halogen atoms, substituted or non-substituted tri(C₁-C₈-alkyl)-silyl, substituted or non-substituted (C₁-C₆-alkylideneamino)oxy, substituted or non-substituted (C₁-C₆-alkenylideneamino)oxy, substituted or non-substituted (C₁-C₆-alkynylideneamino)oxy, substituted or non-substituted (benzylideneamino)oxy; provided that when A represents A² or A¹⁶ and when Z¹, Z², Z³, Z⁴, Z⁵, Z⁶, Z⁷, Z⁸ and Z⁹ independently represent a substituted C₁-C₈-alkylamino or substituted benzylamino, then the substituent is chosen in the list consisting of a pentafluoro-λ⁶-sulphenyl group, a substituted or non-substituted carbaldehyde O—(C₁-C₈-alkyl)oxime, a formylamino group, a (hydroxyimino)-C₁-C₆-alkyl group, a tri(C₁-C₈-alkyl)silyl-C₁-C₈-alkyl, C₃-C₈-cycloalkyl, tri(C₁-C₈-alkyl)silyl-C₁-C₈-cycloalkyl, a C₁-C₈-halogenocycloalkyl having 1 to 5 halogen atoms, C₃-C₈-cycloalkoxy, a C₂-C₈-halogenoalkenyloxy having 1 to 5 halogen atoms, a C₃-C₈-halogenoalkynyloxy having 1 to 5 halogen atoms, a (C₁-C₆-alkoxyimino)-C₁-C₆-alkyl, a (C₁-C₆-alkenyloxyimino)-C₁-C₆-alkyl, a (C₁-C₆-alkynyloxyimino)-C₁-C₆-alkyl, and (benzyloxyimino)-C₁-C₆-alkyl.
K¹ and K² independently represent a hydrogen atom, a formyl group, a substituted or non-substituted carbaldehyde O—(C₁-C₈-alkyl)oxime, a carbamoyl group, a N-hydroxycarbamoyl group, a formylamino group, substituted or non-substituted C₁-C₈-alkyl, substituted or non-substituted tri(C₁-C₈-alkyl)silyl-C₁-C₈-alkyl, substituted or non-substituted C3-C8-cycloalkyl, substituted or non-substituted tri(C1-C8-alkyl)silyl-C3-C8-cycloalkyl, substituted or non-substituted C₁-C₈-halogenoalkyl having 1 to 5 halogen atoms, substituted or non-substituted C3-C8-halogenocycloalkyl having 1 to 5 halogen atoms, a C₂-C₈-alkenyl, substituted or non-substituted C₁-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₂-C₈-alkynyloxy, substituted or non-substituted C₃-C₈-halogenoalkynyloxy having 1 to 5 halogen atoms, substituted or non-substituted C₁-C₈-alkylcarbonyl, substituted or non-substituted N—(C₁-C₈-alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbamothioyl, substituted or non-substituted di-C₁-C₈-alkylcarbamothioyl, substituted or non-substituted N—C₁-C₈-alkyloxycarbamothioyll, substituted or non-substituted C₁-C₈-alkoxycarbamothioyl, substituted or non-substituted N—C₁-C₈alkyl-C₁-C₈-alkoxycarbamothioyl, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-Cᵣhalogenoalkylsulphonyl having 1 to 5 halogen atoms, substituted or non-substituted C₁-C₈-alkylaminosulfamoyl, substituted or non-substituted di-C₁-C₈-alkylaminosulfamoyl, substituted or non-substituted aryl, substituted or non-substituted aryl-[C₁-C₈]-alkyl;
G¹ and G² are independently selected in the list consisting of oxygen, sulfur, NR^{S}, N—OR⁶ and N—NR⁷R⁸;
L¹ represents a direct bond or a divalent group selected in the list consisting of
—(CR¹R²)ₙ— —(CR¹R²)ₘ—C(═O)—(CR¹R²)ₚ—
—(CR¹R²)ₘ—(CR¹═CR²)—(CR¹R²)ₚ— —(CR¹R²)ₘ—C(═O)—O—(CR¹R²)ₚ
—(CR¹R²)ₘ—C≡C—(CR¹R²)ₚ— —(CR¹R²)ₘ—O—C(═O)—(CR¹R²)ₚ—
—(CR¹R²)ₘ—O—(CR¹R²)ₚ— —(CR¹R²)ₘ—C(═O)—NH—(CR¹R²)ₚ—
—(CR¹R²)ₘ—NH—(CR¹R²)ₚ— —(CR¹R²)ₘ—NH—C(═O)—(CR¹R²)ₚ—
wherein
n represents 1, 2, 3 or 4;
m and p independently represent 0, 1, 2 or 3;
L² represents a direct bond or a divalent group selected in the list consisting of
—(CR³R⁴)_{q}—(CR³R⁴)ₐ—C(═O)—(CR³R⁴)_{b}—
—(CR³R⁴)ₐ—(CR³═CR⁴)—(CR³R⁴)_{b}— —(CR³R⁴)ₐ—C(═O)—O—(CR³R⁴)_{b}
—(CR³R⁴)ₐ—C≡C—(CR³R⁴)_{b}— —(CR³R⁴)ₐ—O—C(═O)—(CR³R⁴)_{b}—
—(CR³R⁴)ₐ—O—(CR³R⁴)_{b}— —(CR³R⁴)ₐ—C(═O)—NH—(CR³R⁴)_{b}—
—(CR³R⁴)ₐ—NH—(CR³R⁴)_{b}— —(CR³R⁴)ₐ—NH—C(═O)—(CR³R⁴)_{b}—
wherein
q represents 1, 2, 3 or 4;
a and b independently represent 0, 1, 2 or 3;
T represents a substituted or non-substituted tetrazolyl group;
Q represents a substituted phenyl group of formula (VI) 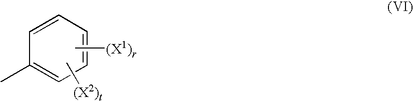
wherein:
X¹ independently represents a nitro group, a hydroxy group, a cyano group, an amino group, a sulphenyl group, a formyl group, a substituted or non-substituted carbaldehyde O—(C₁-C₈-alkyl)oxime, a formyloxy group, a formylamino group, a carbamoyl group, a N-hydroxycarbamoyl group, a pentafluoro-λ⁶-sulphenyl group, a formylamino group, substituted or non-substituted C₁-C₈-alkoxyamino group, substituted or non-substituted N—C₁-C₈-alkyl-(C₁-C₈-alkoxy)-amino group, substituted or non-substituted (C₁-C₈-alkylamino)-amino group, substituted or non-substituted N—C₁-C₈-alkyl-(C₁-C₈-alkylamino)-amino group, a substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C₃-C₈-cycloalkyl, substituted or non-substituted tri(C₁-C₈-alkyl)silyl-C₃-C₈-cycloalkyl, substituted or non-substituted C₁-C₈-halogenoalkyl having 1 to 5 halogen atoms, substituted or non-substituted C3-C8-halogenocycloalkyl having 1 to 5 halogen atoms, a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms, substituted or non-substituted C₁-C₈-alkylsulphenyl, substituted or non-substituted C₁-C₈-halogenoalkylsulphenyl having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms, substituted or non-substituted C₁-C₈-alkylcarbonyl, substituted or non-substituted N—(C₁-C₈-alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈-alkylcarbamoylamino, substituted or non-substituted C₁-C₈-halogenoalkylcarbamoylamino having 1 to 5 halogen atoms, substituted or non-substituted di-C₁-C₈-alkylcarbamoylamino, substituted or non-substituted di-C₁-C₈-halogenoalkylcarbamoylamino having 1 to 5 halogen atoms, substituted or non-substituted N—C₁-C₈-alkyl-(C₁-C₈-alkylcarbamoyl)amino, substituted or non-substituted N—C₁-C₈-alkyl-(C₁-C₈-halogenoalkylcarbamoyl)amino having 1 to 5 halogen atoms, substituted or non-substituted N—C₁-C₈-alkyl-(di-C₁-C₈alkylcarbamoyl)amino, substituted or non-substituted N—C₁-C₈-alkyl-(di-C₁-C₈halogenoalkylcarbamoyl)amino having 1 to 5 halogen atoms, substituted or non-substituted C₁-C₈-alkylaminocarbonyloxy, substituted or non-substituted di-C₁-C₈-alkylaminocarbonyloxy, substituted or non-substituted C₁-C₈-alkylcarbamothioyl, substituted or non-substituted di-C₁-C₈-alkylcarbamothioyl, substituted or non-substituted N—C₁-C₈-alkyloxycarbamothioyll, substituted or non-substituted C₁-C₈-alkoxycarbamothioyl, substituted or non-substituted N—C₁ -C₈-alkyl-C₁-C₈-alkoxycarbamothioyll, substituted or non-substituted C₁-C₈-alkylthioylamino, substituted or non-substituted C₁-C₈-halogenoalkylthioylamino having 1 to 5 halogen atoms, substituted or non-substituted (C₁-C₈-alkyl-carbamothioyl)-oxy, substituted or non-substituted substituted or non-substituted (di-C₁-C₈alkyl-carbamothioyl)-oxy, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkylsulphinyl, substituted or non-substituted C₁-C₈-halogenoalkylsulphinyl having 1 to 5 halogen atoms, substituted or non-substituted C₁-C₈alkylsulphonyl, substituted or non-substituted C₁-C₈-halogenoalkylsulphonyl having 1 to 5 halogen atoms, substituted or non-substituted C₁-C₈-alkylaminosulfamoyl, substituted or non-substituted di-C₁-C₈-alkylaminosulfamoyl, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, substituted or non-substituted (benzyloxyimino)-C₁-C₆-alkyl, substituted or non-substituted benzyloxy, substituted or non-substituted benzylsulphenyl, substituted or non-substituted benzylamino, substituted or non-substituted phenoxy, substituted or non-substituted phenylsulphenyl, substituted or non-substituted phenylamino, substituted or non-substituted aryl, substituted or non-substituted aryl-[C₁-C₈]-alkyl, substituted or non-substituted tri(C₁-C₈alkyl)-silyloxy, substituted or non-substituted C₁-C₈-alkylsulfenylamino, substituted or non-substituted C₁-C₈-halogenoalkylsulphinylamino having 1 to 5 halogen atoms, substituted or non-substituted C₁-C₈-alkylsulphonylamino, substituted or non-substituted C₁-C₈-halogenoalkylsulphonylamino having 1 to 5 halogen atoms, substituted or non-substituted C₁-C₈-alkoxysulphonylamino, substituted or non-substituted C₁-C₈-halogenoxysulphonylamino having 1 to 5 halogen atoms, substituted or non-substituted tri(C₁-C₈-alkyl)-silyl, substituted or non-substituted (C₁-C₆-alkylideneamino)oxy, substituted or non-substituted (C₁-C₆-alkenylideneamino)oxy, substituted or non-substituted (C₁-C₆-alkynylideneamino)oxy, substituted or non-substituted (benzylideneamino)oxy; and
X² independently represents a halogen atom, a nitro group, a hydroxy group, a cyano group, an amino group, a sulphenyl group, a formyl group, a substituted or non-substituted carbaldehyde O—(C₁-C₈-alkyl)oxime, a formyloxy group, a formylamino group, a carbamoyl group, a N-hydroxycarbamoyl group, a pentafluoro-λ⁶-sulphenyl group, a formylamino group, substituted or non-substituted C₁-C₈-alkoxyamino group, substituted or non-substituted N—C₁-C₈-alkyl-(C₁-C₈-alkoxy)-amino group, substituted or non-substituted (C₁-C₈-alkylamino)-amino group, substituted or non-substituted N-C₁-C_{B}alkyl-(C₁-C₈-alkylamino)-amino group, a substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C3-C8-cycloalkyl, substituted or non-substituted tri(C1-C8-alkyl)silyl-C3-C8-cycloalkyl, substituted or non-substituted C₁-C₈-halogenoalkyl having 1 to 5 halogen atoms, substituted or non-substituted C3-C8-halogenocycloalkyl having 1 to 5 halogen atoms, a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms, substituted or non-substituted C₁-C₈-alkylsulphenyl, substituted or non-substituted C₁-C₈-halogenoalkylsulphenyl having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₁-C₈-halogenoalkynyloxy having 1 to 5 halogen atoms, substituted or non-substituted C₁-C₈-alkylcarbonyl, substituted or non-substituted N—(C₁-C₈-alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈-alkylcarbamoylamino, substituted or non-substituted C₁-C₈-halogenoalkylcarbamoylamino having 1 to 5 halogen atoms, substituted or non-substituted di-C₁-C₈-alkylcarbamoylamino, substituted or non-substituted di-C₁-C₈-halogenoalkylcarbamoylamino having 1 to 5 halogen atoms, substituted or non-substituted N—C₁-C₈-alkyl-(C₁-C₈-alkylcarbamoyl)amino, substituted or non-substituted N—C₁-C₈-alkyl-(C₁-C₈halogenoalkylcarbamoyl)amino having 1 to 5 halogen atoms, substituted or non-substituted N—C₁-C₈alkyl-(di-C₁-C₈-alkylcarbamoyl)amino, substituted or non-substituted N—C₁-C₈-alkyl-(di-C₁-C₈-halogenoalkylcarbamoyl)amino having 1 to 5 halogen atoms, substituted or non-substituted C₁-C₈-alkylaminocarbonyloxy, substituted or non-substituted di-C₁-C₈-alkylaminocarbonyloxy, substituted or non-substituted C₁-C₈-alkylcarbamothioyl, substituted or non-substituted di-C₁-C₈-alkylcarbamothioyl, substituted or non-substituted N—C₁-C₈-alkyloxycarbamothioyll, substituted or non-substituted C₁-C₈-alkoxycarbamothioyl, substituted or non-substituted N—C₁-C₈-alkyl-C₁-C₈-alkoxycarbamothioyll, substituted or non-substituted C₁-C₈-alkylthioylamino, substituted or non-substituted C₁-C₈halogenoalkylthioylamino having 1 to 5 halogen atoms, substituted or non-substituted (C₁-C₈alkyl-carbamothioyl)-oxy, substituted or non-substituted substituted or non-substituted (di-C₁-C₈alkyl-carbamothioyl)-oxy, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted C₁-C₈-alkylaminosulfamoyl, substituted or non-substituted di-C₁-C₈-alkylaminosulfamoyl, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, substituted or non-substituted (benzyloxyimino)-C₁-C₆-alkyl, substituted or non-substituted benzyloxy, substituted or non-substituted benzylsulphenyl, substituted or non-substituted benzylamino, substituted or non-substituted phenoxy, substituted or non-substituted phenylsulphenyl, substituted or non-substituted phenylamino, substituted or non-substituted aryl, substituted or non-substituted aryl-[C₁-C₈]-alkyl, substituted or non-substituted tri(C₁-C₈-alkyl)-silyloxy, substituted or non-substituted C₁-C₈-alkylsulfenylamino, substituted or non-substituted C₁-C₈-halogenoalkylsulphinylamino having 1 to 5 halogen atoms substituted or non-substituted C₁-C₈-alkylsulphonylamino, substituted or non-substituted C₁-C₈-halogenoalkylsulphonylamino having 1 to 5 halogen atoms substituted or non-substituted C₁-C₈-alkoxysulphonylamino, substituted or non-substituted C₁-C₈-halogenoxysulphonylamino having 1 to 5 halogen atoms, substituted or non-substituted tri(C₁-C₈-alkyl)-silyl, substituted or non-substituted (C₁-C₆-alkylideneamino)oxy, substituted or non-substituted (C₁-C₆-alkenylideneamino)oxy, substituted or non-substituted (C₁-C₆-alkynylideneamino)oxy, substituted or non-substituted (benzylideneamino)oxy; and
r represents 1, 2, 3, 4 or 5;
t represents 0, 1, 2, 3, 4 or 5 and the sum of r and t equals to 2, 3, 4 or 5;
R¹, R², R³ and R⁴ independently represent a hydrogen atom, a halogen atom, a cyano group, substituted or non-substituted C₁-C₈-alkyl, substituted or non-substituted C3-C8-cycloalkyl, substituted or non-substituted C₁-C₈-halogenoalkyl having 1 to 5 halogen atoms, substituted or non-substituted C3-C8-halogenocycloalkyl having 1 to 5 halogen atoms, a C₂-C₈-alkenyl, substituted or non-substituted C₂-C₈-alkynyl, substituted or non-substituted C₁-C₈-alkoxy, substituted or non-substituted C₁-C₈-halogenoalkoxy having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms,
R⁵ to R⁸ independently represent a hydrogen atom, a halogen atom, a cyano group, substituted or non-substituted C₁-C₈-alkyl, substituted or non-substituted C3-C8-cycloalkyl, substituted or non-substituted C₁-C₈-halogenoalkyl having 1 to 5 halogen atoms, substituted or non-substituted C3-C8-halogenocycloalkyl having 1 to 5 halogen atoms, a C₁-C₈-alkenyl, substituted or non-substituted C₂-C₈-alkynyl, substituted or non-substituted C₁-C₈-alkoxy, substituted or non-substituted C₁-C₈-halogenoalkoxy having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms, substituted or non-substituted aryl, substituted or non-substituted aryl-[C₁-C₈]-alkyl.

23. A compound of formula (IVa) 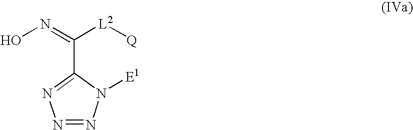
wherein
L² represents a direct bond or a divalent group selected in the list consisting of
—(CR³R⁴)_{g}— —(CR³R⁴)ₐ—C(═O)—(CR³R⁴)_{b}—
—(CR³R⁴)ₐ—(CR³═CR⁴)—(CR³R⁴)_{b}— —(CR³R⁴)ₐ—C(═O)—O—(CR³R⁴)_{b}
—(CR³R⁴)ₐ—C≡C—(CR³R⁴)_{b}— —(CR³R⁴)ₐ—O—C(═O)—(CR³R⁴)_{b}—
—(CR³R⁴)ₐ—O—(CR³R⁴)_{b}— —(CR³R⁴)ₐ—C(═O)—NH—(CR³R⁴)_{b}——(CR³R⁴)ₐ—NH—(CR³R⁴)_{b}— —(CR³R⁴)ₐ—NH—C(═O)—(CR³R⁴)_{b}—
wherein
q represents 1, 2, 3 or 4;
a and b independently represent 0, 1, 2 or 3;
Q represents a substituted phenyl group of formula (VI) 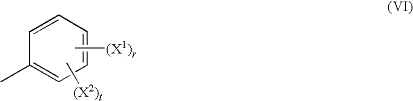
wherein:
X¹ independently represents a nitro group, a hydroxy group, a cyano group, an amino group, a sulphenyl group, a formyl group, a substituted or non-substituted carbaldehyde O—(C₁-C₈-alkyl)oxime, a formyloxy group, a formylamino group, a carbamoyl group, a N-hydroxycarbamoyl group, a pentafluoro-λ⁶-sulphenyl group, a formylamino group, substituted or non-substituted C₁-C₈-alkoxyamino group, substituted or non-substituted N—C₁-C₈-alkyl-(C₁-C₈-alkoxy)-amino group, substituted or non-substituted (C₁-C₈-alkylamino)-amino group, substituted or non-substituted N—C₁-C₈-alkyl-(C₁-C₈-alkylamino)-amino group, a substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C₁-C₈-cycloalkyl, substituted or non-substituted tri(C₁-C₈-alkyl)silyl-C₃-C₈-cycloalkyl, substituted or non-substituted C₁-C₈-halogenoalkyl having 1 to 5 halogen atoms, substituted or non-substituted C3-C8-halogenocycloalkyl having 1 to 5 halogen atoms, a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms, substituted or non-substituted C₁-C₈-alkylsulphenyl, substituted or non-substituted C₁-C₈-halogenoalkylsulphenyl having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms, substituted or non-substituted C₁-C₈-alkylcarbonyl, substituted or non-substituted N—(C₁-C₈-alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈alkylcarbamoylamino, substituted or non-substituted C₁-C₈-halogenoalkylcarbamoylamino having 1 to 5 halogen atoms, substituted or non-substituted di-C₁-C₈-alkylcarbamoylamino, substituted or non-substituted di-C₁-C₈-halogenoalkylcarbamoylamino having 1 to 5 halogen atoms, substituted or non-substituted N—C₁-C₈-alkyl-(C₁-C₈-alkylcarbamoyl)amino, substituted or non-substituted N—C₁-C₈-alkyl-(C₁-C₈-halogenoalkylcarbamoyl)amino having 1 to 5 halogen atoms, substituted or non-substituted N—C₁-C₈-alkyl-(di-C₁-C₈-alkylcarbamoyl)amino, substituted or non-substituted N—C₁-C₈-alkyl-(di-C₁-C₈-halogenoalkylcarbamoyl)amino having 1 to 5 halogen atoms, substituted or non-substituted C₁-C₈-alkylaminocarbonyloxy, substituted or non-substituted di-C₁-C₈-alkylaminocarbonyloxy, substituted or non-substituted C₁-C₈-alkylcarbamothioyl, substituted or non-substituted di-C₁-C₈-alkylcarbamothioyl, substituted or non-substituted N—C₁-C₈-alkyloxycarbamothioyll, substituted or non-substituted C₁-C₈-alkoxycarbamothioyl, substituted or non-substituted N—C₁—C₈ alkyl C₁-C₈-alkoxycarbamothioyll, substituted or non-substituted C₁-C₈-alkylthioylamino, substituted or non-substituted C₁-C₈-halogenoalkylthioylamino having 1 to 5 halogen atoms, substituted or non-substituted (C₁-C₈-alkyl-carbamothioyl)-oxy, substituted or non-substituted substituted or non-substituted (di-C₁-C₈-alkyl-carbamothioyl)-oxy, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted C₁-C₈-alkylaminosulfamoyl, substituted or non-substituted di-C₁-C₈-alkylaminosulfamoyl, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, substituted or non-substituted (benzyloxyimino)-C₁-C₆-alkyl, substituted or non-substituted benzyloxy, substituted or non-substituted benzylsulphenyl, substituted or non-substituted benzylamino, substituted or non-substituted phenoxy, substituted or non-substituted phenylsulphenyl, substituted or non-substituted phenylamino substituted or non-substituted aryl substituted or non-substituted aryl-[C₁-C₈]-alkyl, substituted or non-substituted tri(C₁-C₈-alkyl)-silyloxy, substituted or non-substituted C₁-C₈-alkylsulfenylamino, substituted or non-substituted C₁-C₈-halogenoalkylsulphinylamino having 1 to 5 halogen atoms, substituted or non-substituted C₁-C₈-alkylsulphonylamino, substituted or non-substituted C₁-C₈-halogenoalkylsulphonylamino having 1 to 5 halogen atoms, substituted or non-substituted C₁-C₈-alkoxysulphonylamino, substituted or non-substituted C₁-C₈-halogenoxysulphonylamino having 1 to 5 halogen atoms, substituted or non-substituted tri(C₁-C₈-alkyl)-silyl, substituted or non-substituted (C₁-C₆-alkylideneamino)oxy, substituted or non-substituted (C₁-C₆-alkenylideneamino)oxy, substituted or non-substituted (C₁-C₆-alkynylideneamino)oxy, substituted or non-substituted (benzylideneamino)oxy; and
X² independently represents a halogen atom, a nitro group, a hydroxy group, a cyano group, an amino group, a sulphenyl group, a formyl group, a substituted or non-substituted carbaldehyde O—(C₁-C₈-alkyl)oxime, a formyloxy group, a formylamino group, a carbamoyl group, a N-hydroxycarbamoyl group, a pentafluoro-λ⁶-sulphenyl group, a formylamino group, substituted or non-substituted C₁-C₈-alkoxyamino group, substituted or non-substituted N—C₁-C₈-alkyl-(C₁-C₈-alkoxy)-amino group, substituted or non-substituted C₁-C₈-alkylamino)-amino group, substituted or non-substituted N—C₁-C₈-alkyl-(C₁-C₈-alkylamino)-amino group, a substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C3-C8-cycloalkyl, substituted or non-substituted tri(C1-C8-alkyl)silyl-C3-C8-cycloalkyl, substituted or non-substituted C₁-C₈-halogenoalkyl having 1 to 5 halogen atoms, substituted or non-substituted C3-C8-halogenocycloalkyl having 1 to 5 halogen atoms, a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms, substituted or non-substituted C₁-C₈-alkylsulphenyl, substituted or non-substituted C₁-C₈-halogenoalkylsulphenyl having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms, substituted or non-substituted C₁-C₈-alkylcarbonyl, substituted or non-substituted N—(C₁-C₈-alkoxy)-C₁-C₈alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈alkylcarbonylamino, substituted or non-substituted C₁-C₈-halogenoalkylcarbonylamino having 1 to 5 halogen atoms, substituted or non-substituted C₁-C₈-alkylcarbamoylamino, substituted or non-substituted C₁-C₈-halogenoalkylcarbamoylamino having 1 to 5 halogen atoms, substituted or non-substituted di-C₁-C₈-alkylcarbamoylamino, substituted or non-substituted di-C₁-C₈-halogenoalkylcarbamoylamino having 1 to 5 halogen atoms , substituted or non-substituted N—C₁-C₈-alkyl-(C₁-C₈-alkylcarbamoyl)amino, substituted or non-substituted N—C₁-C₈-alkyl-(C₁-C₈-halogenoalkylcarbamoyl)amino having 1 to 5 halogen atoms, substituted or non-substituted N—C₁-C₈-alkyl-(di-C₁-C₈-alkylcarbamoyl)amino, substituted or non-substituted N—C₁-C₈-alkyl-(di-C₁-C₈-halogenoalkylcarbamoyl)amino having 1 to 5 halogen atoms, substituted or non-substituted C₁-C₈-alkylaminocarbonyloxy, substituted or non-substituted di-C₁-C₈alkylaminocarbonyloxy, substituted or non-substituted C₁-C₈-alkylcarbamothioyl, substituted or non-substituted di-C₁-C₈-alkylcarbamothioyl, substituted or non-substituted N—C₁-C₈-alkyloxycarbamothioyll, substituted or non-substituted C₁-C₈-alkoxycarbamothioyl, substituted or non-substituted N—C₁-C₈-alkyl-C₁-C₈-alkoxycarbamothioyll, substituted or non-substituted C₁-C₈-alkylthioylamino, substituted or non-substituted C₁-C₈-halogenoalkylthioylamino having 1 to 5 halogen atoms, substituted or non-substituted (C₁-C₈-alkyl-carbamothioyl)-oxy, substituted or non-substituted substituted or non-substituted (di-C₁-C₈-alkyl-carbamothioyl)-oxy, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted C₁-C₈-alkylaminosulfamoyl, substituted or non-substituted di-C₁-C₈-alkylaminosulfamoyl, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, substituted or non-substituted (benzyloxyimino)-C₁-C₆-alkyl, substituted or non-substituted benzyloxy, substituted or non-substituted benzylsulphenyl, substituted or non-substituted benzylamino, substituted or non-substituted phenoxy, substituted or non-substituted phenylsulphenyl, substituted or non-substituted phenylamino, substituted or non-substituted aryl, substituted or non-substituted aryl-[C₁-C₈]-alkyl, substituted or non-substituted tri(C₁-C₈-alkyl)-silyloxy, substituted or non-substituted C₁-C₈-alkylsulfenylamino, substituted or non-substituted C₁-C₈-halogenoalkylsulphinylamino having 1 to 5 halogen atoms, substituted or non-substituted C₁-C₈-alkylsulphonylamino, substituted or non-substituted C₁-C₈-halogenoalkylsulphonylamino having 1 to 5 halogen atoms, substituted or non-substituted C₁-C₈-alkoxysulphonylamino, substituted or non-substituted C₁-C₈-halogenoxysulphonylamino having 1 to 5 halogen atoms, substituted or non-substituted tri(C₁-C₈-alkyl)-silyl, substituted or non-substituted (C₁-C₆-alkylideneamino)oxy, substituted or non-substituted (C₁-C₆-alkenylideneamino)oxy, substituted or non-substituted (C₁-C₆-alkynylideneamino)oxy, substituted or non-substituted (benzylideneamino)oxy; and
r represents 1, 2, 3, 4 or 5;
t represents 0, 1, 2, 3, 4 or 5 and the sum of r and t equals to 2, 3, 4 or 5;
E¹ represents a hydrogen atom, a formyl group, a substituted or non-substituted carbaldehyde O—(C₁-C₈-alkyl)oxime, a carbamoyl group, a N-hydroxycarbamoyl group, a substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C3-C8-cycloalkyl, substituted or non-substituted tri(C₁-C8-alkyl)silyl-C3-C8-cycloalkyl, substituted or non-substituted C₁-C₈-halogenoalkyl having 1 to 5 halogen atoms, substituted or non-substituted C3-C8-halogenocycloalkyl having 1 to 5 halogen atoms, a C₇-C₈-alkenyl, substituted or non-substituted C₇-C₈-alkynyl, substituted or non-substituted C₁-C₈-alkylcarbonyl, substituted or non-substituted N—(C₁-C₈-alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈alkoxycarbonyl, substituted or non-substituted C₁-C₈-halogenoalkoxycarbonyl having 1 to 5 halogen atoms, substituted or non-substituted C₁-C₈-alkylcarbamothioyl, substituted or non-substituted di-C₁-C₈-alkylcarbamothioyl, substituted or non-substituted N—C₁-C₈-alkyloxycarbamothioyl, substituted or non-substituted C₁-C₈-alkoxycarbamothioyl, substituted or non-substituted N—C₁-C₈-alkyl-C₁-C₈-alkoxycarbamothioyl, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted C₁-C₈-alkylaminosulfamoyl, substituted or non-substituted di-C₁-C₈-alkylaminosulfamoyl, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, substituted or non-substituted (benzyloxyimino)-C₁-C₆-alkyl, substituted or non-substituted aryl, substituted or non-substituted aryl-[C₁-C₈]-alkyl, substituted or non-substituted tri(C₁-C₈-alkyl)-silyl;
R¹, R², R³ and R⁴ independently represent a hydrogen atom, a halogen atom, a cyano group, substituted or non-substituted C₁-C₈-alkyl, substituted or non-substituted C3-C8-cycloalkyl, substituted or non-substituted C₁-C₈-halogenoalkyl having 1 to 5 halogen atoms, substituted or non-substituted C3-C8-halogenocycloalkyl having 1 to 5 halogen atoms, a C₇-C₈-alkenyl, substituted or non-substituted C₂-C₈-alkynyl, substituted or non-substituted C₁-C₈-alkoxy, substituted or non-substituted C₁-C₈-halogenoalkoxy having 1 to 5 halogen atoms, substituted or non-substituted C₂-C₈alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms.

24. A compound of formula (IVb): 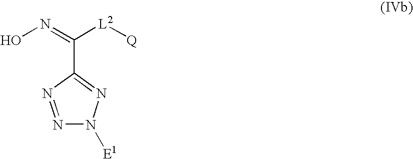
wherein
L² represents a direct bond or a divalent group selected in the list consisting of
—(CR³R⁴)ₐ—(CR³═R⁴)ₐ—C(═O)—(CR³R⁴)_{b}—
—(CR³R⁴)ₐ—(CR³═CR⁴)—(CR³R⁴)_{b}— —(CR³R⁴)ₐ—C(═O)—O—(CR³R⁴)_{b}
—(CR³R⁴)ₐ—C≡C—(CR³R⁴)_{b}— —(CR³R⁴)ₐ—O—C(═O)—(CR³R⁴)_{b}—
—(CR³R⁴)ₐ—O—(CR³R⁴)_{b}— —(CR³R⁴)ₐ—C(═O)—NH—(CR³R⁴)_{b}—
—(CR³R⁴)ₐ—NH—(CR³R⁴)_{b}— —(CR³R⁴)ₐ—NH—C(═O)—(CR³R⁴)_{b}—
wherein
q represents 1, 2, 3 or 4;
a and b independently represent 0, 1, 2 or 3;
Q represents a substituted phenyl group of formula (VI) 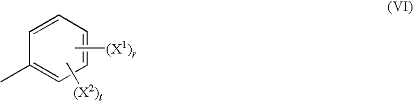
wherein:
X¹ independently represents a nitro group, a hydroxy group, a cyano group, an amino group, a sulphenyl group, a formyl group, a substituted or non-substituted carbaldehyde O-(C₁-C₈-alkyl)oxime, a formyloxy group, a formylamino group, a carbamoyl group, a N-hydroxycarbamoyl group, a pentafluoro-λ⁶-sulphenyl group, a formylamino group, substituted or non-substituted C₁-C₈-alkoxyamino group, substituted or non-substituted N—C₁-C₈-alkyl-(C₁-C₈-alkoxy)-amino group, substituted or non-substituted (C₁-C₈-alkylamino)-amino group, substituted or non-substituted N—C₁-C₈-alkyl-(C₁-C₈-alkylamino)-amino group, a substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C₃-C₈-cycloalkyl, substituted or non-substituted tri(C₁-C₈-alkyl)silyl-C₃-C₈-cycloalkyl, substituted or non-substituted C₁-C₈-halogenoalkyl having 1 to 5 halogen atoms, substituted or non-substituted C3-C8-halogenocycloalkyl having 1 to 5 halogen atoms, a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms, substituted or non-substituted C₁-C₈-alkylsulphenyl, substituted or non-substituted C₁-C₈-halogenoalkylsulphenyl having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms, substituted or non-substituted C₁-C₈-alkylcarbonyl, substituted or non-substituted N—(C₁-C₈-alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈alkylcarbonyloxy, substituted or non-substituted C₁-C₈-halogenoalkylcarbonyloxy having 1 to 5 halogen atoms, substituted or non-substituted C₁-C₈alkylcarbonylamino, substituted or non-substituted C₁-C₈-halogenoalkylcarbonylamino having 1 to 5 halogen atoms, substituted or non-substituted C₁-C₈-alkylcarbamoylamino, substituted or non-substituted C₁-C₈-halogenoalkylcarbamoylamino having 1 to 5 halogen atoms, substituted or non-substituted di-C₁-C₈-alkylcarbamoylamino, substituted or non-substituted di-C₁-C₈-halogenoalkylcarbamoylamino having 1 to 5 halogen atoms, substituted or non-substituted N—C₁-C₈-alkyl-(C₁-C₈-alkylcarbamoyl)amino, substituted or non-substituted N—C₁-C₈-alkyl-(C₁-C₈halogenoalkylcarbamoyl)amino having 1 to 5 halogen atoms, substituted or non-substituted N—C₁-C₈-alkyl-(di-C₁-C₈-alkylcarbamoyl)amino, substituted or non-substituted N—C₁-C₈-alkyl-(di-C₁-C₈-halogenoalkylcarbamoyl)amino having 1 to 5 halogen atoms, substituted or non-substituted C₁-C₈-alkylaminocarbonyloxy, substituted or non-substituted di-C₁-C₈-alkylaminocarbonyloxy, substituted or non-substituted C₁-C₈alkylcarbamothioyl, substituted or non-substituted di-C₁-C₈-alkylcarbamothioyl, substituted or non-substituted N—C₁-C₈-alkyloxycarbamothioyll, substituted or non-substituted C₁-C₈-alkoxycarbamothioyl, substituted or non-substituted N—C₁-C₈-alkyl-C₁-C₈-alkoxycarbamothioyll, substituted or non-substituted C₁-C₈-alkylthioylamino, substituted or non-substituted C₁-C₈-halogenoalkylthioylamino having 1 to 5 halogen atoms, substituted or non-substituted (C₁-C₈-alkyl-carbamothioyl)-oxy, substituted or non-substituted substituted or non-substituted (di-C₁-C₈-alkyl-carbamothioyl)-oxy, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted C₁-C₈-alkylaminosulfamoyl, substituted or non-substituted di-C₁-C₈-alkylaminosulfamoyl, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, substituted or non-substituted (benzyloxyimino)-C₁-C₆-alkyl, substituted or non-substituted benzyloxy, substituted or non-substituted benzylsulphenyl, substituted or non-substituted benzylamino, substituted or non-substituted phenoxy, substituted or non-substituted phenylsulphenyl, substituted or non-substituted phenylamino, substituted or non-substituted aryl, substituted or non-substituted aryl-[C₁-C₈]-alkyl, substituted or non-substituted tri(C₁-C₈-alkyl)-silyloxy, substituted or non-substituted C₁-C₈-alkylsulfenylamino, substituted or non-substituted C₁-C₈-halogenoalkylsulphinylamino having 1 to 5 halogen atoms, substituted or non-substituted C₁-C₈-alkylsulphonylamino, substituted or non-substituted C₁-C₈-halogenoalkylsulphonylamino having 1 to 5 halogen atoms, substituted or non-substituted C₁-C₈alkoxysulphonylamino, substituted or non-substituted C₁-C₈-halogenoxysulphonylamino having 1 to 5 halogen atoms, substituted or non-substituted tri(C₁-C₈-alkyl)-silyl, substituted or non-substituted (C₁-C₆-alkylideneamino)oxy, substituted or non-substituted (C₁-C₆-alkenylideneamino)oxy, substituted or non-substituted (C₁-C₆-alkynylideneamino)oxy, substituted or non-substituted (benzylideneamino)oxy; and
X² independently represents a halogen atom, a nitro group, a hydroxy group, a cyano group, an amino group, a sulphenyl group, a formyl group, a substituted or non-substituted carbaldehyde O—(C₁-C₈-alkyl)oxime, a formyloxy group, a formylamino group, a carbamoyl group, a N-hydroxycarbamoyl group, a pentafluoro-λ⁶-sulphenyl group, a formylamino group, substituted or non-substituted C₁-C₈-alkoxyamino group, substituted or non-substituted N—C₁-C₈-alkyl-(C₁-C₈-alkoxy)-amino group, substituted or non-substituted (C₁-C₈-alkylamino)-amino group, substituted or non-substituted N—C₁-C₈-alkyl-(C₁-C₈-alkylamino)-amino group, a substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C3-C8-cycloalkyl, substituted or non-substituted tri(C1-C8-alkyl)silyl-C3-C8-cycloalkyl, substituted or non-substituted C₁-C₈-halogenoalkyl having 1 to 5 halogen atoms, substituted or non-substituted C3-C8-halogenocycloalkyl having 1 to 5 halogen atoms, a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms, substituted or non-substituted C₁-C₈-alkylsulphenyl, substituted or non-substituted C₁-C₈-halogenoalkylsulphenyl having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms, substituted or non-substituted C₁-C₈-alkylcarbonyl, substituted or non-substituted N—(C₁-C₈-alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈-alkylcarbamoylamino, substituted or non-substituted C₁-C₈-halogenoalkylcarbamoylamino having 1 to 5 halogen atoms, substituted or non-substituted di-C₁-C₈-alkylcarbamoylamino, substituted or non-substituted di-C₁-C₈-halogenoalkylcarbamoylamino having 1 to 5 halogen atoms, substituted or non-substituted N—C₁-C₈-alkyl-(C₁-C₈-alkylcarbamoyl)amino, substituted or non-substituted N—C₁-C₈-alkyl-(C₁-C₈-halogenoalkylcarbamoyl)amino having 1 to 5 halogen atoms, substituted or non-substituted N—C₁-C₈-alkyl-(di-C₁-C₈-alkylcarbamoyl)amino, substituted or non-substituted N—C₁-C₈-alkyl-(di-C₁-C₈-halogenoalkylcarbamoyl)amino having 1 to 5 halogen atoms, substituted or non-substituted C₁-C₈-alkylaminocarbonyloxy, substituted or non-substituted di-C₁-C₈-alkylaminocarbonyloxy, substituted or non-substituted C₁-C₈-alkylcarbamothioyl, substituted or non-substituted di-C₁-C₈-alkylcarbamothioyl, substituted or non-substituted N—C₁-C₈-alkyloxycarbamothioyll, substituted or non-substituted C₁-C₈-alkoxycarbamothioyl, substituted or non-substituted N—C ₁-C₈-alkyl-C₁-C₈-alkoxycarbamothioyll, substituted or non-substituted C₁-C₈-alkylthioylamino, substituted or non-substituted C₁-C₈-halogenoalkylthioylamino having 1 to 5 halogen atoms, substituted or non-substituted (C₁-C₈-alkyl-carbamothioyl)-oxy, substituted or non-substituted substituted or non-substituted (di-C₁-C₈-alkyl-carbamothioyl)-oxy, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted C₁-C₈-alkylaminosulfamoyl, substituted or non-substituted di-C₁-C₈-alkylaminosulfamoyl, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, substituted or non-substituted (benzyloxyimino)-C₁-C₆-alkyl, substituted or non-substituted benzyloxy, substituted or non-substituted benzylsulphenyl, substituted or non-substituted benzylamino, substituted or non-substituted phenoxy, substituted or non-substituted phenylsulphenyl, substituted or non-substituted phenylamino, substituted or non-substituted aryl, substituted or non-substituted aryl-[C₁-C₈]-alkyl, substituted or non-substituted tri(C₁-C₈-alkyl)-silyloxy, substituted or non-substituted C₁-C₈-alkylsulfenylamino, substituted or non-substituted C₁-C₈-halogenoalkylsulphinylamino having 1 to 5 halogen atoms, substituted or non-substituted C₁-C₈alkylsulphonylamino, substituted or non-substituted C₁-C₈-halogenoalkylsulphonylamino having 1 to 5 halogen atoms, substituted or non-substituted C₁-C₈-alkoxysulphonylamino, substituted or non-substituted C₁-C₈-halogenoxysulphonylamino having 1 to 5 halogen atoms, substituted or non-substituted tri(C₁-C₈-alkyl)-silyl, substituted or non-substituted (C₁-C₆-alkylideneamino)oxy, substituted or non-substituted (C₁-C₆-alkenylideneamino)oxy, substituted or non-substituted (C₁-C₆-alkynylideneamino)oxy, substituted or non-substituted (benzylideneamino)oxy; and
r represents 1, 2, 3, 4 or 5;
t represents 0, 1, 2, 3, 4 or 5 and the sum of r and t equals to 2, 3, 4 or 5;
E¹ represents a hydrogen atom, a formyl group, a substituted or non-substituted carbaldehyde O—(C₁-C₈-alkyl)oxime, a carbamoyl group, a N-hydroxycarbamoyl group, a substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C3-C8-cycloalkyl, substituted or non-substituted tri(C1-C8-alkyl)silyl-C3-C8-cycloalkyl, substituted or non-substituted C₁-C₈-halogenoalkyl having 1 to 5 halogen atoms, substituted or non-substituted C3-C8-halogenocycloalkyl having 1 to 5 halogen atoms, a C₂-C₈-alkenyl, substituted or non-substituted C₂-C₈-alkynyl, substituted or non-substituted C₁-C₈-alkylcarbonyl, substituted or non-substituted N—(C₁-C₈-alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbamothioyl, substituted or non-substituted di-C₁-C₈-alkylcarbamothioyl, substituted or non-substituted N—C₁-C₈alkyloxycarbamothioyl, substituted or non-substituted C₁-C₈-alkoxycarbamothioyl, substituted or non-substituted N—C₁-C₈-alkyl-C₁-C₈-alkoxycarbamothioyl, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted C₁-C₈-alkylaminosulfamoyl, substituted or non-substituted di-C₁-C₈-alkylaminosulfamoyl, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, substituted or non-substituted (benzyloxyimino)-C₁-C₆-alkyl, substituted or non-substituted aryl, substituted or non-substituted aryl-[C₁-C₈]-alkyl, substituted or non-substituted tri(C₁-C₈-alkyl)-silyl;
R¹, R², R³, and R⁴ independently represent a hydrogen atom, a halogen atom, a cyano group, substituted or non-substituted C₁-C₈-alkyl, substituted or non-substituted C3-C8-cycloalkyl, substituted or non-substituted C₁-C₈-halogenoalkyl having 1 to 5 halogen atoms, substituted or non-substituted C3-C8-halogenocycloalkyl having 1 to 5 halogen atoms, a C₂-C₈-alkenyl, substituted or non-substituted C₂-C₈-alkynyl, substituted or non-substituted C₁-C₈-alkoxy, substituted or non-substituted C₁-C₈-halogenoalkoxy having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms.

25. A compound of formula (Va) 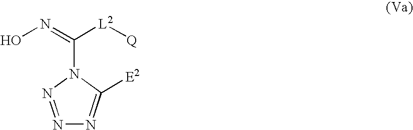
wherein
L² represents a direct bond or a divalent group selected in the list consisting of
—(CR³R⁴)ₐ— —(CR³R⁴)ₐ—C(═O)—(CR³R⁴)_{b}—
—(CR³R⁴)ₐ—(CR³═CR⁴)—(CR³R⁴)_{b}— —(CR³R⁴)ₐ—C(═O)—O—(CR³R⁴)_{b}
—(CR³R⁴)ₐ—C≡C—(CR³R⁴)_{b}— —(CR³R⁴)ₐ—O—C(═O)—(CR³R⁴)_{b}—
—(CR³R⁴)ₐ—O—(CR³R⁴)_{b}— —(CR³R⁴)ₐ—C(═O)—NH—(CR³R⁴)_{b}—
—(CR³R⁴)ₐ—NH—(CR³R⁴)_{b}— —(CR³R⁴)ₐ—NH—C(═O)—(CR³R⁴)_{b}—
wherein
q represents 1, 2, 3 or 4;
a and b independently represent 0, 1, 2 or 3;
Q represents a substituted phenyl group of formula (VI) 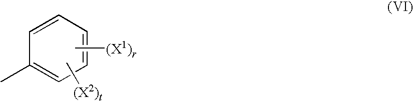
wherein:
X¹ independently represents a nitro group, a hydroxy group, a cyano group, an amino group, a sulphenyl group, a formyl group, a substituted or non-substituted carbaldehyde O—(C₁-C₈-alkyl)oxime, a formyloxy group, a formylamino group, a carbamoyl group, a N-hydroxycarbamoyl group, a pentafluoro-λ⁶-sulphenyl group, a formylamino group, substituted or non-substituted C₁-C₈-alkoxyamino group, substituted or non-substituted N—C₁-C₈-alkyl-(C₁-C₈-alkoxy)-amino group, substituted or non-substituted (C₁-C₈-alkylamino)-amino group, substituted or non-substituted N—C₁-C₈-alkyl-(C₁-C₈-alkylamino)-amino group, a substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C₃-C₈-cycloalkyl, substituted or non-substituted tri(C₁-C₈-alkyl)silyl-C₃-C₈-cycloalkyl, substituted or non-substituted C₁-C₈-halogenoalkyl having 1 to 5 halogen atoms, substituted or non-substituted C3-C8-halogenocycloalkyl having 1 to 5 halogen atoms, a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms, substituted or non-substituted C₁-C₈-alkylsulphenyl, substituted or non-substituted C₁-C₈-halogenoalkylsulphenyl having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms, substituted or non-substituted C₁-C₈-alkylcarbonyl, substituted or non-substituted N—(C₁-C₈-alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈alkyl-C₁-C₈alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈-alkylcarbamoylamino, substituted or non-substituted C₁-C₈-halogenoalkylcarbamoylamino having 1 to 5 halogen atoms, substituted or non-substituted di-C₁-C₈-alkylcarbamoylamino, substituted or non-substituted halogenoalkylcarbamoylamino having 1 to 5 halogen atoms, substituted or non-substituted N—C₁-C₈-alkyl-(C₁-C₈alkylcarbamoyl)amino, substituted or non-substituted N—C₁-C₈-alkyl-(C₁-C₈halogenoalkylcarbamoyl)amino having 1 to 5 halogen atoms, substituted or non-substituted N—C₁-C₈-alkyl-(di-C₁-C₈-alkylcarbamoyl)amino, substituted or non-substituted N—C₁-C₈-alkyl-(di-C₁-C₈-halogenoalkylcarbamoyl)amino having 1 to 5 halogen atoms, substituted or non-substituted C₁-C₈-alkylaminocarbonyloxy, substituted or non-substituted di-C₁-C₈-alkylaminocarbonyloxy, substituted or non-substituted C₁-C₈-alkylcarbamothioyl, substituted or non-substituted di-C₁-C₈-alkylcarbamothioyl, substituted or non-substituted N—C₁-C₈-alkyloxycarbamothioyll, substituted or non-substituted C₁-C₈-alkoxycarbamothioyl, substituted or non-substituted N—C₁-C₈-alkyl-C₁-C₈-alkoxycarbamothioyll, substituted or non-substituted C₁-C₈-alkylthioylamino, substituted or non-substituted C₁-C₈-halogenoalkylthioylamino having 1 to 5 halogen atoms, substituted or non-substituted (C₁-C₈-alkyl-carbamothioyl)-oxy, substituted or non-substituted substituted or non-substituted (di-C₁-C₈-alkyl-carbamothioyl)-oxy, substituted or non-substituted C₁-C₈-alkyl sulphenyl, substituted or non-substituted C₁-C₈-halogenoalkylsulphenyl having 1 to 5 halogen atoms, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted C₁-C₈-alkylaminosulfamoyl, substituted or non-substituted di-C₁-C₈-alkylaminosulfamoyl, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, substituted or non-substituted (benzyloxyimino)-C₁-C₆-alkyl, substituted or non-substituted benzyloxy, substituted or non-substituted benzylsulphenyl, substituted or non-substituted benzylamino, substituted or non-substituted phenoxy, substituted or non-substituted phenyl sulphenyl substituted or non-substituted phenylamino, substituted or non-substituted aryl, substituted or non-substituted aryl-[C₁-C₈]-alkyl, substituted or non-substituted tri(C₁-C₈-alkyl)-silyloxy, substituted or non-substituted C₁-C₈-alkylsulfenylamino, substituted or non-substituted C₁-C₈-halogenoalkylsulphinylamino having 1 to 5 halogen atoms, substituted or non-substituted C₁-C₈-alkylsulphonylamino, substituted or non-substituted C₁-C₈-halogenoalkylsulphonylamino having 1 to 5 halogen atoms, substituted or non-substituted C₁-C₈-alkoxysulphonylamino, substituted or non-substituted C₁-C₈-halogenoxysulphonylamino having 1 to 5 halogen atoms, substituted or non-substituted tri(C₁-C₈-alkyl)-silyl, substituted or non-substituted (C₁-C₆-alkylideneamino)oxy, substituted or non-substituted (C₁-C₆-alkenylideneamino)oxy, substituted or non-substituted (C₁-C₆-alkynylideneamino)oxy, substituted or non-substituted (benzylideneamino)oxy; and
X² independently represents a halogen atom, a nitro group, a hydroxy group, a cyano group, an amino group, a sulphenyl group, a formyl group, a substituted or non-substituted carbaldehyde O—(C₁-C₈-alkyl)oxime, a formyloxy group, a formylamino group, a carbamoyl group, a N-hydroxycarbamoyl group, a pentafluoro-λ⁶-sulphenyl group, a formylamino group, substituted or non-substituted C₁-C₈-alkoxyamino group, substituted or non-substituted N—C₁-C₈-alkyl-(C₁-C₈-alkoxy)-amino group, substituted or non-substituted (C₁-C₈-alkylamino)-amino group, substituted or non-substituted N—C₁-C₈-alkyl-(C₁-C₈-alkylamino)-amino group, a substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C3-C8-cycloalkyl, substituted or non-substituted tri(C1-C8-alkyl)silyl-C3-C8-cycloalkyl, substituted or non-substituted C₁-C₈-halogenoalkyl having 1 to 5 halogen atoms, substituted or non-substituted C3-C8-halogenocycloalkyl having 1 to 5 halogen atoms, a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms, substituted or non-substituted C₁-C₈-alkylsulphenyl, substituted or non-substituted C₁-C₈-halogenoalkylsulphenyl having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms, substituted or non-substituted C₁-C₈-alkylcarbonyl, substituted or non-substituted N—(C₁-C₈-alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈-alkylcarbamoylamino, substituted or non-substituted C₁-C₈-halogenoalkylcarbamoylamino having 1 to 5 halogen atoms, substituted or non-substituted di-C₁-C₈-alkylcarbamoylamino, substituted or non-substituted di-C₁-C₈-halogenoalkylcarbamoylamino having 1 to 5 halogen atoms, substituted or non-substituted N—C₁-C₈-alkyl-(C₁-C₈-alkylcarbamoyl)amino, substituted or non-substituted N—C₁-C₈-alkyl-(C₁-C₈-halogenoalkylcarbamoyl)amino having 1 to 5 halogen atoms, substituted or non-substituted N—C₁-C₈-alkyl-(di-C₁-C₈-alkylcarbamoyl)amino, substituted or non-substituted N—C₁-C₈-alkyl-(di-C₁-C₈-halogenoalkylcarbamoyl)amino having 1 to 5 halogen atoms, substituted or non-substituted C₁-C₈alkylaminocarbonyloxy, substituted or non-substituted di-C₁-C₈-alkylaminocarbonyloxy, substituted or non-substituted C₁-C₈-alkylcarbamothioyl, substituted or non-substituted di-C₁-C₈-alkylcarbamothioyl, substituted or non-substituted N—C₁-C₈-alkyloxycarbamothioyll, substituted or non-substituted C₁-C₈-alkoxycarbamothioyl, substituted or non-substituted N—C₁-C₈-alkyl-C₁-C₈-alkoxycarbamothioyll, substituted or non-substituted C₁-C₈-alkylthioylamino, substituted or non-substituted C₁-C₈-halogenoalkylthioylamino having 1 to 5 halogen atoms, substituted or non-substituted (C₁-C₈-alkyl-carbamothioyl)-oxy, substituted or non-substituted substituted or non-substituted (di-C₁-C₈-alkyl-carbamothioyl)-oxy, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted C₁-C₈-alkylaminosulfamoyl, substituted or non-substituted di-C₁-C₈-alkylaminosulfamoyl, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, substituted or non-substituted (benzyloxyimino)-C₁-C₆-alkyl, substituted or non-substituted benzyloxy, substituted or non-substituted benzylsulphenyl, substituted or non-substituted benzylamino, substituted or non-substituted phenoxy, substituted or non-substituted phenylsulphenyl substituted or non-substituted phenylamino, substituted or non-substituted aryl, substituted or non-substituted aryl-[C₁-C₈]-alkyl, substituted or non-substituted tri(C₁-C₈-alkyl)-silyloxy, substituted or non-substituted C₁-C₈-alkylsulfenylamino, substituted or non-substituted C₁-C₈-halogenoalkylsulphinylamino having 1 to 5 halogen atoms, substituted or non-substituted C₁-C₈-alkylsulphonylamino, substituted or non-substituted C₁-C₈-halogenoalkylsulphonylamino having 1 to 5 halogen atoms, substituted or non-substituted C₁-C₈-alkoxysulphonylamino, substituted or non-substituted C₁-C₈-halogenoxysulphonylamino having 1 to 5 halogen atoms, substituted or non-substituted tri(C₁-C₈-alkyl)-silyl, substituted or non-substituted (C₁-C₆-alkylideneamino)oxy, substituted or non-substituted (C₁-C₆-alkenylideneamino)oxy, substituted or non-substituted (C₁-C₆-alkynylideneamino)oxy, substituted or non-substituted (benzylideneamino)oxy; and
r represents 1, 2, 3, 4 or 5
t represents 0, 1, 2, 3, 4 or 5 and the sum of r and t equals to 2, 3, 4 or 5;
E² represents a hydrogen atom, a halogen atom, a nitro group, a hydroxy group, a cyano group, an amino group, a sulphenyl group, a formyl group, a substituted or non-substituted carbaldehyde O—(C₁-C₈-alkyl)oxime, a formyloxy group, a formylamino group, a carbamoyl group, a N-hydroxycarbamoyl group, a pentafluoro-λ⁶-sulphenyl group, a formylamino group, substituted or non-substituted C₁-C₈-alkoxyamino group, substituted or non-substituted N—C₁-C₈-alkyl-(C₁-C₈-alkoxy)-amino group, substituted or non-substituted (C₁-C₈-alkylamino)-amino group, substituted or non-substituted N—C₁-C₈-alkyl-(C₁-C₈-alkylamino)-amino group, a substituted or non-substituted (hydroxyimino)-C₁-C₆-alkyl group, substituted or non-substituted C₁-C₈-alkyl, substituted or non-substituted tri(C₁-C_{g}-alkyl)silyl-C₁-C₈-alkyl, substituted or non-substituted C3-C8-cycloalkyl, substituted or non-substituted tri(C1-C8-alkyl)silyl-C3-C8-cycloalkyl, substituted or non-substituted C₁-C₈-halogenoalkyl having 1 to 5 halogen atoms, substituted or non-substituted C3-C8-halogenocycloalkyl having 1 to 5 halogen atoms, a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms, substituted or non-substituted C₁-C₈-alkylsulphenyl, substituted or non-substituted C₁-C₈-halogenoalkylsulphenyl having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₁-C₈-halogenoalkynyloxy having 1 to 5 halogen atoms, substituted or non-substituted C₁-C₈-alkylcarbonyl, substituted or non-substituted N—(C₁-C₈-alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈alkylcarbamoylamino, substituted or non-substituted C₁-C₈-halooallyclcarbamoylamino having 1 to 5 halogen atoms, substituted or non-substituted di-C₁-C₈-alkylcarbamoylamino, substituted or non-substituted di-C₁-C₈-halogenoalkylcarbamoylamino having 1 to 5 halogen atoms, substituted or non-substituted N—C₁-C₈-alkyl-(C₁-C₈-alkylcarbamoyl)amino, substituted or non-substituted N—C₁-C₈-alkyl-(C₁-C₈-halogenoalkylcarbamoyl)amino having 1 to 5 halogen atoms, substituted or non-substituted N—C₁-C₈-alkyl-(di-C₁-C₈-alkylcarbamoyl)amino, substituted or non-substituted N—C₁-C₈-alkyl-(di-C₁-C₈-halogenoalkylcarbamoyl)amino having 1 to 5 halogen atoms, substituted or non-substituted C₁-C₈-alkylaminocarbonyloxy, substituted or non-substituted di-C₁-C₈-alkylaminocarbonyloxy, substituted or non-substituted C₁-C₈-alkylcarbamothioyl, substituted or non-substituted di-C₁-C₈-alkylcarbamothioyl, substituted or non-substituted N—C₁-C₈-alkyloxycarbamothioyll, substituted or non-substituted C₁-C₈-alkoxycarbamothioyl, substituted or non-substituted N—C₁-C₈-alkyl-C₁-C₈-alkoxycarbamothioyll, substituted or non-substituted C₁-C₈-alkylthioylamino, substituted or non-substituted C₁-C₈-halogenoalkylthioylamino having 1 to 5 halogen atoms, substituted or non-substituted (C₁-C₈-alkyl-carbamothioyl)-oxy, substituted or non-substituted substituted or non-substituted (di-C₁-C₈-alkyl-carbamothioyl)-oxy, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted C₁-C₈-alkylaminosulfamoyl, substituted or non-substituted di-C₁-C₈-alkylaminosulfamoyl, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, substituted or non-substituted (benzyloxyimino)-C₁-C₆-alkyl, substituted or non-substituted benzyloxy, substituted or non-substituted benzylsulphenyl, substituted or non-substituted benzylamino, substituted or non-substituted phenoxy, substituted or non-substituted phenylsulphenyl, substituted or non-substituted phenylamino, substituted or non-substituted aryl, substituted or non-substituted aryl-[C₁-C₈]-alkyl, substituted or non-substituted tri(C₁-C₈-alkyl)-silyloxy, substituted or non-substituted C₁-C₈-alkyl sulfenylamino, substituted or non-substituted halogenoalkylsulphinylamino having 1 to 5 halogen atoms, substituted or non-substituted C₁-C₈-alkylsulphonylamino, substituted or non-substituted C₁-C₈-halogenoalkylsulphonylamino having 1 to 5 halogen atoms, substituted or non-substituted C₁-C₈-alkoxysulphonylamino, substituted or non-substituted C₁-C₈-halogenoxysulphonylamino having 1 to 5 halogen atoms, substituted or non-substituted tri(C₁-C₈-alkyl)-silyl, substituted or non-substituted (C₁-C₆-alkylideneamino)oxy, substituted or non-substituted (C₁-C₆-alkenylideneamino)oxy, substituted or non-substituted (C₁-C₆-alkynylideneamino)oxy, substituted or non-substituted (benzylideneamino)oxy;
R¹, R², R³ and R⁴ independently represent a hydrogen atom, a halogen atom, a cyano group, substituted or non-substituted C₁-C₈-alkyl, substituted or non-substituted C3-C8-cycloalkyl, substituted or non-substituted C₁-C₈-halogenoalkyl having 1 to 5 halogen atoms, substituted or non-substituted C3-C8-halogenocycloalkyl having 1 to 5 halogen atoms, a C₂-C₈-alkenyl, substituted or non-substituted C₂-C₈-alkynyl, substituted or non-substituted C₁-C₈alkoxy, substituted or non-substituted C₁-C₈-halogenoalkoxy having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₁-C₈-alkynyloxy, substituted or non-substituted C₃-C₈-halogenoalkynyloxy having 1 to 5 halogen atoms.

26. A compound of formula (Vb) 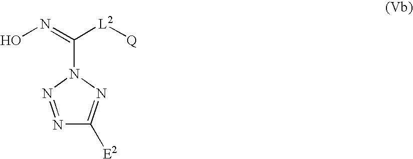
wherein
L² represents a direct bond or a divalent group selected in the list consisting of
—(CR³R⁴)ₐ— —(CR³R⁴)ₐ—C(═O)—(CR³R⁴)_{b}—
—(CR³R⁴)ₐ—(CR³═CR⁴)—(CR³R⁴)_{b}— —(CR³R⁴)ₐ—C(═O)—O—(CR³R⁴)_{b}
—(CR³R⁴)ₐ—C≡C—(CR³R⁴)_{b}— —(CR³R⁴)ₐ—O—C(═O)—(CR³R⁴)_{b}—
—(CR³R⁴)ₐ—O—(CR³R⁴)_{b}— —(CR³R⁴)ₐ—C(═O)—NH—(CR³R⁴)_{b}—
—(CR³R⁴)ₐ—NH—(CR³R⁴)_{b}— —(CR³R⁴)ₐ—NH—C(═O)—(CR³R⁴)_{b}—
wherein
q represents 1, 2, 3 or 4 ,
a and b independently represent 0, 1, 2 or 3;
Q represents a substituted phenyl group of formula (VI) 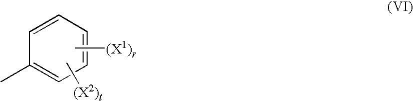
wherein:
X¹ independently represents a nitro group, a hydroxy group, a cyano group, an amino group, a sulphenyl group, a formyl group, a substituted or non-substituted carbaldehyde O-(C₁-C₈-alkyl)oxime, a formyloxy group, a formylamino group, a carbamoyl group, a N-hydroxycarbamoyl group, a pentafluoro-λ⁶-sulphenyl group a formylamino group, substituted or non-substituted C₁-C₈-alkoxyamino group, substituted or non-substituted N—C₁-C₈-alkyl-(C₁-C₈-alkoxy)-amino group, substituted or non-substituted (C₁-C₈-alkylamino)-amino group, substituted or non-substituted N—C₁-C₈-alkyl-(C₁-C₈-alkylamino)-amino group, a substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C₃-C₈-cycloalkyl, substituted or non-substituted tri(C₁-C₈-alkyl)silyl-C₃-C₈-cycloalkyl, substituted or non-substituted C₁-C₈-halogenoalkyl having 1 to 5 halogen atoms, substituted or non-substituted C3-C8-halogenocycloalkyl having 1 to 5 halogen atoms, a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms, substituted or non-substituted C₁-C₈-alkylsulphenyl, substituted or non-substituted C₁-C₈-halogenoalkylsulphenyl having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms, substituted or non-substituted C₁-C₈-alkylcarbonyl, substituted or non-substituted N—(C₁-C₈alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈-alkylcarbamoylamino, substituted or non-substituted C₁-C₈-halogenoalkylcarbamoylamino having 1 to 5 halogen atoms substituted or non-substituted di-C₁-C₈-alkylcarbamoylamino, substituted or non-substituted di-C₁-C₈-halogenoalkylcarbamoylamino having 1 to 5 halogen atoms, substituted or non-substituted N—C₁-C₈-alkyl-(C₁-C₈-alkylcarbamoyl)amino, substituted or non-substituted N—C₁-C₈-alkyl-(C₁-C₈-halogenoalkylcarbamoyl)amino having 1 to 5 halogen atoms, substituted or non-substituted N—C₁-C₈alkyl-(di-C₁-C₈alkylcarbamoyl)amino, substituted or non-substituted N—C₁-C₈-alkyl-(di-C₁-C₈-halogenoalkylcarbamoyl)amino having 1 to 5 halogen atoms, substituted or non-substituted C₁-C₈-alkylaminocarbonyloxy, substituted or non-substituted di-C₁-C₈-alkylaminocarbonyloxy, substituted or non-substituted C₁-C₈alkylcarbamothioyl, substituted or non-substituted di-C₁-C₈-alkylcarbamothioyl, substituted or non-substituted N—C₁-C₈-alkyloxycarbamothioyll, substituted or non-substituted C₁-C₈-alkoxycarbamothioyl, substituted or non-substituted N—C₁-C₈-alkyl-C₁-C₈-alkoxycarbamothioyll, substituted or non-substituted C₁-C₈-alkylthioylamino, substituted or non-substituted C₁-C₈-halogenoalkylthioylamino having 1 to 5 halogen atoms, substituted or non-substituted (C₁-C₈-alkyl-carbamothioyl)-oxy, substituted or non-substituted substituted or non-substituted (di-C₁-C₈-alkyl-carbamothioyl)-oxy, substituted or non-substituted C₁-C₈-alkyl sulphenyl, substituted or non-substituted C₁-C₈-halogenoalkylsulphenyl having 1 to 5 halogen atoms, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted C₁-C₈-alkylaminosulfamoyl, substituted or non-substituted di-C₁-C₈-alkylaminosulfamoyl, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, substituted or non-substituted (benzyloxyimino)-C₁-C₆-alkyl, substituted or non-substituted benzyloxy, substituted or non-substituted benzylsulphenyl, substituted or non-substituted benzylamino, substituted or non-substituted phenoxy, substituted or non-substituted phenylsulphenyl, substituted or non-substituted phenylamino, substituted or non-substituted aryl, substituted or non-substituted aryl-[C₁-C₈]-alkyl, substituted or non-substituted tri(C₁-C₈alkyl)-silyloxy, substituted or non-substituted C₁-C₈-alkylsulfenylamino, substituted or non-substituted C₁-C₈-halogenoalkylsulphinylamino having 1 to 5 halogen atoms, substituted or non-substituted C₁-C₈-alkylsulphonylamino, substituted or non-substituted C₁-C₈-halogenoalkylsulphonylamino having 1 to 5 halogen atoms, substituted or non-substituted C₁-C₈-alkoxysulphonylamino, substituted or non-substituted C₁ -C₈-halogenoxysulphonylamino having 1 to 5 halogen atoms, substituted or non-substituted tri(C₁-C₈-alkyl)-silyl, substituted or non-substituted (C₁-C₆-alkylideneamino)oxy, substituted or non-substituted (C₁-C₆-alkenylideneamino)oxy, substituted or non-substituted (C₁-C₆-alkynylideneamino)oxy, substituted or non-substituted (benzylideneamino)oxy; and
X² independently represents a halogen atom, a nitro group, a hydroxy group, a cyano group, an amino group, a sulphenyl group, a formyl group, a substituted or non-substituted carbaldehyde O-(C₁-C₈-alkynoxime, a formyloxy group, a formylamino group, a carbamoyl group, a N-hydroxycarbamoyl group, a pentafluoro-λ⁶-sulphenyl group, a formylamino group, substituted or non-substituted C₁-C₈-alkoxyamino group, substituted or non-substituted N—C₁-C₈-alkyl-(C₁-C₈-alkoxy)-amino group, substituted or non-substituted (C₁-C₈-alkylamino)-amino group, substituted or non-substituted N—C₁-C₈-alkyl-(C₁-C₈-alkylamino)-amino group, a substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C3-C8-cycloalkyl, substituted or non-substituted tri(C1-C8-alkyl)silyl-C3-C8-cycloalkyl, substituted or non-substituted C₁-C₈-halogenoalkyl having 1 to 5 halogen atoms, substituted or non-substituted C3-C8-halogenocycloalkyl having 1 to 5 halogen atoms, a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms, substituted or non-substituted C₁-C₈-alkyl sulphenyl, substituted or non-substituted C₁-C₈-halogenoalkylsulphenyl having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms, substituted or non-substituted C₁-C₈-alkylcarbonyl, substituted or non-substituted N—(C₁-C₈-alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈-alkylcarbamoylamino, substituted or non-substituted C₁-C₈-halogenoalkylcarbamoylamino having 1 to 5 halogen atoms, substituted or non-substituted di-C₁-C₈-alkylcarbamoylamino, substituted or non-substituted halogenoalkylcarbamoylamino having 1 to 5 halogen atoms, substituted or non-substituted N—C₁-C₈-alkyl-(C₁-C₈-alkylcarbamoynamino, substituted or non-substituted N—C₁-C₈-alkyl-(C₁-C₈-halogenoalkylcarbamoyl)amino having 1 to 5 halogen atoms, substituted or non-substituted N—C₁-C₈-alkyl-(di-C₁-C₈-alkylcarbamoynamino, substituted or non-substituted N—C₁-C₈-alkyl-(di-C₁-C₈-halogenoalkylcarbamoyl)amino having 1 to 5 halogen atoms, substituted or non-substituted C₁-C₈-alkylaminocarbonyloxy, substituted or non-substituted di-C₁-C₈alkylaminocarbonyloxy, substituted or non-substituted C₁-C₈-alkylcarbamothioyl, substituted or non-substituted di-C₁-C₈-alkylcarbamothioyl, substituted or non-substituted N—C₁-C₈alkyloxycarbamothioyll, substituted or non-substituted alkoxycarbamothioyl, substituted or non-substituted N—C₁-C₈-alkyl-C₁-C₈-alkoxycarbamothioyll, substituted or non-substituted C₁-C₈-alkylthioylamino, substituted or non-substituted C₁-C₈-halogenoalkylthioylamino having 1 to 5 halogen atoms, substituted or non-substituted (C₁-C₈-alkyl-carbamothioyl)-oxy, substituted or non-substituted substituted or non-substituted (di-C₁-C₈-alkyl-carbamothioyl)-oxy, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkylsulphinyl, substituted or non-substituted C₁-C₈halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted C₁-C₈-alkylaminosulfamoyl, substituted or non-substituted di-C₁-C₈-alkylaminosulfamoyl, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, substituted or non-substituted (benzyloxyimino)-C₁-C₆-alkyl, substituted or non-substituted benzyloxy, substituted or non-substituted benzylsulphenyl, substituted or non-substituted benzylamino, substituted or non-substituted phenoxy, substituted or non-substituted phenylsulphenyl, substituted or non-substituted phenylamino, substituted or non-substituted aryl, substituted or non-substituted aryl-[C₁-C₈]-alkyl, substituted or non-substituted tri(C₁-C₈-alkyl)-silyloxy, substituted or non-substituted C₁-C₈-alkylsulfenylamino, substituted or non-substituted C₁-C₈-halogenoalkylsulphinylamino having 1 to 5 halogen atoms, substituted or non-substituted C₁-C₈-alkylsulphonylamino, substituted or non-substituted halogenoalkylsulphonylamino having 1 to 5 halogen atoms, substituted or non-substituted C₁-C₈-alkoxysulphonylamino, substituted or non-substituted C₁-C₈-halogenoxysulphonylamino having 1 to 5 halogen atoms, substituted or non-substituted tri(C₁-C₈-alkyl)-silyl, substituted or non-substituted (C₁-C₆-alkylideneamino)oxy, substituted or non-substituted (C₁-C₆-alkenylideneamino)oxy, substituted or non-substituted (C₁-C₆-alkynylideneamino)oxy, substituted or non-substituted (benzylideneamino)oxy; and
r represents 1, 2, 3, 4 or 5;
t represents 0, 1, 2, 3, 4 or 5 and the sum of r and t equals to 2, 3, 4 or 5;
E² represents a hydrogen atom, a halogen atom, a nitro group, a hydroxy group, a cyano group, an amino group, a sulphenyl group, a formyl group, a substituted or non-substituted carbaldehyde O—(C₁-C₈-alkyl)oxime, a formyloxy group, a formylamino group, a carbamoyl group, a N-hydroxycarbamoyl group, a pentafluoro-λ⁶-sulphenyl group, a formylamino group, substituted or non-substituted C₁-C₈-alkoxyamino group, substituted or non-substituted N—C₁-C₈-alkyl-(C₁-C₈-alkoxy)-amino group, substituted or non-substituted (C₁-C₈-alkylamino)-amino group, substituted or non-substituted N—C₁-C₈-alkyl-(C₁-C₈-alkylamino)-amino group, a substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈alkyl, substituted or non-substituted C3-C8-cycloalkyl, substituted or non-substituted tri(C1-C8-alkyl)silyl-C3-C8-cycloalkyl, substituted or non-substituted C₁-C₈-halogenoalkyl having 1 to 5 halogen atoms, substituted or non-substituted C3-C8-halogenocycloalkyl having 1 to 5 halogen atoms, a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-Cₛ-alkoxy, substituted or non-substituted C₁-C₈-halogenoalkoxy having 1 to 5 halogen atoms, substituted or non-substituted C₁-C₈-alkylsulphenyl, substituted or non-substituted C₁-C₈-halogenoalkylsulphenyl having 1 to 5 halogen atoms, substituted or non-substituted C₇-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₁-C₈-halogenoalkynyloxy having 1 to 5 halogen atoms, substituted or non-substituted C₁-C₈-alkylcarbonyl, substituted or non-substituted N—(C₁-C₈-alkoxy)-C₁-C₈-alkanimidoyl, substituted or non-substituted N—(C₁-C₈-alkoxy)-C₁-C₈-halogenoalkanimidoyl having 1 to 5 halogen atoms, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈-alkylcarbamoylamino, substituted or non-substituted C₁-C₈-halogenoalkylcarbamoylamino having 1 to 5 halogen atoms, substituted or non-substituted di-C₁-C₈-alkylcarbamoylamino, substituted or non-substituted halogenoalkylcarbamoylamino having 1 to 5 halogen atoms, substituted or non-substituted N—C₁-C₈alkyl-(C₁-C₈-alkylcarbamoyl)amino, substituted or non-substituted N—C₁-C₈alkyl-(C₁-C₈-halogenoalkylcarbamoyl)amino having 1 to 5 halogen atoms, substituted or non-substituted N—C₁-C₈-alkyl-(di-C₁-C₈-alkylcarbamoyl)amino, substituted or non-substituted N—C₁-C₈-alkyl-(di-C₁-C₈-halogenoalkylcarbamoyl)amino having 1 to 5 halogen atoms, substituted or non-substituted C₁-C₈-alkylaminocarbonyloxy, substituted or non-substituted di-C₁-C₈-alkylaminocarbonyloxy, substituted or non-substituted C₁-C₈-alkylcarbamothioyl, substituted or non-substituted di-C₁-C₈alkylcarbamothioyl, substituted or non-substituted N—C₁-C₈-alkyloxycarbamothioyll, substituted or non-substituted C₁-C₈-alkoxycarbamothioyl, substituted or non-substituted N—C₁-C₈-alkyl-C₁-C₈-alkoxycarbamothioyll, substituted or non-substituted C₁-C₈-alkylthioylamino, substituted or non-substituted C₁-C₈-halogenoalkylthioylamino having 1 to 5 halogen atoms, substituted or non-substituted (C₁-C₈-alkyl-carbamothioyl)-oxy, substituted or non-substituted substituted or non-substituted (di-C₁-C₈-alkyl-carbamothioyl)-oxy, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted C₁-C₈-alkylaminosulfamoyl, substituted or non-substituted di-C₁-C₈-alkylaminosulfamoyl, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, substituted or non-substituted (benzyloxyimino)-C₁-C₆-alkyl, substituted or non-substituted benzyloxy, substituted or non-substituted benzylsulphenyl, substituted or non-substituted benzylamino, substituted or non-substituted phenoxy, substituted or non-substituted phenylsulphenyl, substituted or non-substituted phenylamino, substituted or non-substituted aryl, substituted or non-substituted aryl-[C₁-C₈]-alkyl, substituted or non-substituted tri(C₁-C₈-alkyl)-silyloxy, substituted or non-substituted C₁-C₈-alkylsulfenylamino, substituted or non-substituted C₁-C₈-halogenoalkylsulphinylamino having 1 to 5 halogen atoms, substituted or non-substituted C₁-C₈-alkylsulphonylamino, substituted or non-substituted C₁-C₈-halogenoalkylsulphonylamino having 1 to 5 halogen atoms, substituted or non-substituted C₁-C₈-alkoxysulphonylamino, substituted or non-substituted C₁-C₈-halogenoxysulphonylamino having 1 to 5 halogen atoms, substituted or non-substituted tri(C₁-C₈-alkyl)-silyl, substituted or non-substituted (C₁-C₆-alkylideneamino)oxy, substituted or non-substituted (C₁-C₆-alkenyl ideneamino)oxy, substituted or non-substituted (C₁-C₆-alkynylideneamino)oxy, substituted or non-substituted (benzylideneamino)oxy;
R¹, R², R³ and R⁴ independently represent a hydrogen atom, a halogen atom, a cyano group, substituted or non-substituted C₁-C₈-alkyl, substituted or non-substituted C3-C8-cycloalkyl, substituted or non-substituted C₁-C₈-halogenoalkyl having 1 to 5 halogen atoms, substituted or non-substituted C3-C8-halogenocycloalkyl having 1 to 5 halogen atoms, a C₁-C₈-alkenyl, substituted or non-substituted C₂-C₈-alkynyl, substituted or non-substituted C₁-C₈-alkoxy, substituted or non-substituted C₁-C₈-halogenoalkoxy having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms.

27. A method for controlling the phytopathogenic fungi or damaging insects of plants, crops or seeds comprising the application of an agronomically effective and substantially non-phytotoxic quantity of a pesticide composition according to claim 1 as seed treatment, foliar application, stem application, drench or drip application or chemigation to the seed, the plant or to the fruit of the plant or to soil or to inert substrate, Pumice, Pyroclastic materials or stuff, synthetic organic substrates organic substrates or to a liquid substrate wherein the plant is growing or wherein it is desired to grow.
